(19) United States
(12) Patent Application Publication
(10) Pub. No.: US 2024/0141389 A1
(43) Pub. Date: May 2, 2024

(54) THERAPEUTIC GENOME EDITING IN WISKOTT-ALDRICH SYNDROME AND X-LINKED THROMBOCYTOPENIA

(71) Applicant: Seattle Children's Hospital (dba Seattle Children's Research Institute), Seattle, WA (US)

(72) Inventors: David J. Rawlings, Seattle, WA (US); Iram Khan, Issaquah, WA (US)

(21) Appl. No.: 18/296,235

(22) Filed: Apr. 5, 2023

Related U.S. Application Data
(63) Continuation of application No. 16/605,748, filed on Oct. 16, 2019, now Pat. No. 11,643,671.
(60) Provisional application No. 62/488,249, filed on Apr. 21, 2017.

(51) Int. Cl. C12N 15/90; C12N 9/22

(52) U.S. Cl. CPC C12N 15/907; C12N 9/22

(57) ABSTRACT

Described herein are systems and methods for treating, inhibiting, or ameliorating X-linked disorders including Wiskott-Aldrich Syndrome (WAS) and X-linked thrombocytopenia (XLT) in subjects that have been identified or selected as being ones that would benefit from a therapy to treat, inhibit, or ameliorate WAS or XLT. The systems include nuclease and vector donor constructs configured for co-delivery to modify endogenous WAS locus.

### CROSS REFERENCE TO RELATED APPLICATIONS

This application is a continuation of U.S. application Ser. No. 16/605,748 filed Oct. 16, 2019 which is the U.S. National Phase of PCT Int. App. No. PCT/US2018/028442, filed on Apr. 19, 2018, designating the United States of America and published in the English language as WO 2018/195360, which claims priority to U.S. Prov. App. No. 62/488,249, filed on Apr. 21, 2017, which are each expressly incorporated by reference in its entirety.

### REFERENCE TO SEQUENCE LISTING

The present application is being filed along with a Sequence Listing in electronic format. The Sequence Listing is provided as a file entitled SCRI149C1SEQLIST, created Apr. 5, 2023, which is approximately 182471 bytes in size. The information provided in the electronic format of the Sequence Listing is incorporated herein by reference in its entirety.

### FIELD

Alternatives herein relate generally to gene editing in primary human hematopoietic stem cells. More particularly, alternatives herein relate to nucleic acids and vectors that are configured to provide efficient homology directed repair of genes, and methods of repairing genetic deficiencies, such as X-linked recessive disorders.

### BACKGROUND

Wiskott-Aldrich syndrome (WAS) is an inherited primary immunodeficiency caused by mutations in the WAS gene, which encodes a protein that regulates the actin cytoskeleton leading to altered signaling function and/or development in multiple hematopoietic cell lineages. WAS is an X-linked recessive disease, affecting approximately one in 250,000 males. The disease is characterized by eczema, thrombocytopenia, and immune deficiency. WAS is caused by a mutation in the WAS gene, resulting in mutated or absent forms of the WAS protein (WASp).

X-linked thrombocytopenia (XLT) is an inherited clotting disorder related to WAS. XLT primarily affects the circulatory system by reducing the platelet counts in the blood, and also reducing the platelet size compared to normal or healthy individuals, which compromises the clotting process. XLT is caused by a mutation in the WAS gene, resulting in decreased, absent, or altered WASp.

WASp is an activator of the actin nucleator Arp2/3 complex in vitro and is expressed exclusively in hematopoietic cells. WASp is believed to serve as a key integrator between surface receptors and the cytoskeleton of leukocytes.

Currently, therapies for individuals with WAS or XLT focus on the symptoms. The only available curative therapy for WAS or XLT is allogeneic stem cell transplantation. Viral-based gene replacement is also being explored in several current clinical trials but the need for more therapies for WAS and XLT is manifest.

### SUMMARY

Some alternatives relate to therapeutic approaches designed to correct or repair the endogenous WAS locus in autologous hematopoietic stem cells. Some alternatives include compositions and methods, which comprise transcription activator-like effector nucleases (TALENs) and clustered regularly interspaced short palindromic repeats (CRISPR)/Cas that are configured to edit the WAS locus in primary human hematopoietic cells. Some alternatives also relate to co-delivery of a nuclease and an AAV donor for modifying endogenous WAS locus in primary human hematopoietic cells.

Some alternatives concern a nucleic acid for homology directed repair (HDR) of the Wiskott-Aldrich Syndrome (WAS) gene. In some alternatives, the nucleic acid comprises a first sequence encoding a WAS gene, a second sequence encoding one or more guide RNA cleavage sites, and a third sequence encoding one or more nuclease binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nuclease binding sites is a clustered regularly interspaced short palindromic repeats (CRISPR) associated protein 9 (Cas9) binding site. In some alternatives, the nucleic acid further comprises one or more enhancer elements. In some alternatives, the nucleic acid further comprises homology arm sequences. In some alternatives, the nucleic acid further comprises a nucleic acid sequence encoding a promoter.

Some alternatives relate to a vector for promoting HDR of WAS protein (WASp) expression in a cell. In some alternatives, the vector comprises a first sequence encoding a WAS gene, a second sequence encoding one or more guide RNA cleavage sites, and a third sequence encoding one or more nuclease binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the vector further comprises one or more enhancer elements. In some alternatives, the vector is an adeno-associated viral vector (AAV). In some alternatives, the vector is a self-complementary AAV (scAAV). In some alternatives, the cell is a human cell. In some alternatives, the cell is a primary cell. In some alternatives, the cell is an autologous cell. In some alternatives, the cell is a T cell. In some alternatives, the cell is a hematopoietic stem cell (HSC). In some alternatives, the cell is a CD34⁺ HSC.

Some alternatives relate to a system for promoting HDR of WAS protein (WASp) expression in a cell. In some alternatives, the system comprises a vector and a nucleic acid encoding a nuclease. In some alternatives, the nuclease is a TALEN nuclease. In some alternatives, the nuclease is a Cas nuclease. In some alternatives, the vector and nucleic acid are configured for co-delivery to the cell. In some alternatives, co-delivery to the cell modifies endogenous WAS locus. In some alternatives, the cell is a primary human hematopoietic cell.

Some alternatives relate to a cell for expressing a WASp. In some alternatives, the cell comprises a nucleic acid. In some alternatives, the nucleic acid comprises a first sequence encoding a WAS gene, a second sequence encoding a promoter, a third sequence encoding one or more guide RNA cleavage sites, and a fourth sequence encoding one or more nuclease binding sites. In some alternatives, the nucleic acid is in a vector. In some alternatives, the vector is an AAV. In some alternatives, the vector is a scAAV. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34⁺ HSC.

Some alternatives relate to methods of promoting HDR of a WAS gene in a subject in need thereof (e.g., a subject identified or selected as one that would receive a benefit from HDR of a WAS gene, such as a subject having XLT). In some alternatives, the method comprises administering to a subject in need thereof a cell as described herein or a vector as described herein and administering to the subject a nuclease. In some alternatives, the nuclease is a TALEN nuclease. In some alternatives, the nuclease is a Cas nuclease. In some alternatives, the nuclease is co-administered to the subject with the cell or with the vector. In some alternatives, the cell is from the subject. In some alternatives, the cell is genetically modified by introducing a nucleic acid as described herein or a vector as described herein into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34⁺ HSC. In some alternatives, the subject is male. In some alternatives, the subject is identified or selected as one that is suffering from Wiskott-Aldrich syndrome (WAS). In some alternatives, the subject is identified or selected as one that is suffering from X-linked thrombocytopenia (XLT).

Some alternatives provided herein relate to a method of treating, inhibiting, or ameliorating WAS and/or XLT or disease symptoms associated with WAS and/or XLT in a subject in need thereof. In some alternatives, the method comprises administering to a subject a cell as described herein or a vector as described herein, administering to the subject a nuclease, and optionally identifying or selecting the subject as one that would benefit from receiving a therapy for WAS and/or XLT or disease symptoms associated with WAS and/or XLT and/or, optionally measuring an improvement in the progression of WAS and/or XLT or an improvement in a disease symptom associated with WAS and/or XLT in said subject. In some alternatives, the nuclease is a TALEN nuclease. In some alternatives, the nuclease is a CRISPR/Cas nuclease. In some alternatives, the nuclease is co-administered to the subject with the cell or with the vector. In some alternatives, the cell is from the subject. In some alternatives, the cell is genetically modified by introducing a nucleic acid as described herein or a vector as described herein into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34⁺ HSC In some alternatives, the subject is male. In some alternatives, the method improves thrombocytopenia. In some alternatives, the method increases platelet counts. In some alternatives, the subject is identified or selected as one that is suffering from Wiskott-Aldrich syndrome (WAS). In some alternatives, the subject is identified or selected as one that is suffering from X-linked thrombocytopenia (XLT).

In a first aspect, a nucleic acid for homology directed repair (HDR) of Wiskott-Aldrich Syndrome (WAS) gene is provided, the nucleic acid comprising: a first sequence encoding a WAS gene; a second sequence encoding one or more guide RNA cleavage sites; and a third sequence encoding one or more nuclease binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the nucleic acid further comprises one or more enhancer elements. In some alternatives, the nucleic acid further comprises homology arm sequences. In some alternatives, the nucleic acid further comprises a nucleic acid sequence encoding a promoter.

In a second aspect, a vector for promoting HDR of WAS protein (WASp) expression in a cell is provided, the vector comprising: a first sequence encoding a WAS gene; a second sequence encoding one or more guide RNA cleavage sites; and a third sequence encoding one or more nuclease binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the vector further comprises one or more enhancer elements. In some alternatives, the vector is an adeno-associated viral vector (AAV). In some alternatives, the vector is a self-complementary AAV (scAAV). In some alternatives, the cell is a human cell. In some alternatives, the cell is a primary cell. In some alternatives, the cell is an autologous cell. In some alternatives, the cell is a T cell. In some alternatives, the cell is a hematopoietic stem cell (HSC). In some alternatives, the cell is a CD34+ HSC.

In a third aspect, a system for promoting HDR of WAS protein (WASp) expression in a cell is provided, the system comprising a vector of any one of the alternatives herein and a nucleic acid encoding a nuclease. In some alternatives, the nuclease is a TALEN nuclease. In some alternatives, the nuclease is a Cas nuclease. In some alternatives, the vector and nucleic acid are configured for co-delivery to the cell. In some alternatives, co-delivery to the cell modifies endogenous WAS locus. In some alternatives, the cell is a primary human hematopoietic cell.

In a fourth aspect, a cell for expressing a WASp is provided, the cell comprising: a nucleic acid, which comprises: a first sequence encoding a WAS gene; a second sequence encoding a promoter; a third sequence encoding one or more guide RNA cleavage sites; and a fourth sequence encoding one or more nuclease binding sites. In some alternatives, the nucleic acid is in a vector. In some alternatives, the vector is an AAV. In some alternatives, the AAV is a scAAV. In some alternatives, cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34+ HSC.

In a fifth aspect, a method of promoting HDR of a WAS gene in a subject in need thereof, the method comprising: administering to a subject the cell or a vector of any one of the alternatives herein; and administering to the subject a nuclease. The cell comprises: a nucleic acid, which comprises: a first sequence encoding a WAS gene; a second sequence encoding a promoter; a third sequence encoding one or more guide RNA cleavage sites; and a fourth sequence encoding one or more nuclease binding sites. The vector comprises: a first sequence encoding a WAS gene; a second sequence encoding one or more guide RNA cleavage sites; and a third sequence encoding one or more nuclease binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the vector further comprises one or more enhancer elements. In some alternatives, the vector is an adeno-associated viral vector (AAV). In some alternatives, the vector is a self-complementary AAV (scAAV). In some alternatives, the cell is a human cell. In some alternatives, the cell is a primary cell. In some alternatives, the cell is an autologous cell. In some alternatives, the cell is a T cell. In some alternatives, the cell is a hematopoietic stem cell (HSC). In some alternatives, the cell is a CD34+ HSC. In some alternatives, the nucleic acid is in a vector. In some alternatives, the vector is an AAV. In some alternatives, the AAV is a scAAV. In some alternatives, cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34+ HSC In some alternatives, the nuclease is a TALEN nuclease. In some alternatives, the nuclease is a Cas nuclease. In some alternatives, the nuclease is co-administered to the subject with the cell or with the vector. In some alternatives, the cell is from the subject and, wherein the cell is genetically modified by introducing the nucleic acid or the vector of any one of the alternatives described herein into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34+ HSC. In some alternatives, the subject is male. In some alternatives, the subject is suffering from Wiskott-Aldrich syndrome (WAS). In some alternatives, the subject is suffering from X-linked thrombocytopenia (XLT).

In a sixth aspect, a method of treating, inhibiting, or ameliorating WAS and/or XLT or disease symptoms associated with WAS and/or XLT in a subject in need thereof, the method comprising: administering to a subject the cell or a vector of any one of the alternatives described herein; administering to the subject a nuclease; and optionally identifying the subject as one that would benefit from receiving a therapy for WAS and/or XLT or disease symptoms associated with WAS and/or XLT and/or, optionally measuring an improvement in the progression of WAS and/or XLT or an improvement in a disease symptom associated with WAS and/or XLT in said subject. The cell comprises: a nucleic acid, which comprises: a first sequence encoding a WAS gene; a second sequence encoding a promoter; a third sequence encoding one or more guide RNA cleavage sites; and a fourth sequence encoding one or more nuclease binding sites. The vector comprises: a first sequence encoding a WAS gene; a second sequence encoding one or more guide RNA cleavage sites; and a third sequence encoding one or more nuclease binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the vector further comprises one or more enhancer elements. In some alternatives, the vector is an adeno-associated viral vector (AAV). In some alternatives, the vector is a self-complementary AAV (scAAV). In some alternatives, the nuclease is a TALEN nuclease. the nuclease is a CRISPR/Cas nuclease. the nuclease is co-administered to the subject with the cell or with the vector. the cell is from the subject, wherein the cell is genetically modified by introducing the nucleic acid or the vector of any one of the alternatives described herein into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34+ HSC. In some alternatives, the subject is male. In some alternatives, the method improves thrombocytopenia. In some alternatives, the method increases platelet counts.

### BRIEF DESCRIPTION OF THE DRAWINGS

FIGS. 1A and 1B depict the assessment of cleavage efficiency of TALENs and guide RNAs in primary T cells. FIG. 1A shows a schematic of the WAS locus with guides and TALENs annotated. Shown are the location of the WAS TALENs (TALEN #1 (T1) and TALEN #2 (T2)) and guide RNA (G1, G2, G3, and G4) cleavage sites within the human WAS gene. Scheme not drawn to scale. FIG. 1B depicts the disruption of the WAS locus with TALENs and CRISPR guides. Shown is the percentage of NHEJ events in primary human T cells detected using the T7 assay, 5 days post-transfection with TALEN mRNAs (T1, T2) or with Cas9 mRNA co-delivered with scAAV expressing gRNAs (G1-4). N=3, represents the number of independent experiments. TALEN #1 and G1 were selected for all further experiments.

FIGS. 2A-2I depict human primary T cell editing with TALENs and rationally designed AAV donor templates. FIG. 2A shows a schematic representation of the WAS locus and AAV donor templates used in targeting with WAS TALENs. Small boxes represent TALEN forward (T-for) and reverse (T-rev) binding sites. Homology arms are also depicted. X represents mutation of the T preceding the TALEN binding site designed to abolish cleavage by TALEN in #1244 vector. The #1262 vector has the entire region between the exon 1 up to the reverse TALEN binding site deleted. FIG. 2B depicts a timeline of gene editing procedure beginning with bead stimulation of primary T cells. AAVs were added at 20% of culture volume. FIGS. 2C and 2D depict time course of GFP expression indicative of HDR (FIG. 2C) and cell viability (FIG. 2D). FIG. 2E shows cells that were also transfected with TALENs and transduced with an AAV vector with an MND promoter driven blue fluorescent protein (BFP) without any homology arms. Fluorescence from this vector at day 15 is indicative of random integration of the vector. Fluorescence from this vector at Day 15 is indicative of random integration of the vector. FIG. 2F shows representative FACS plots with GFP expression at day 15 post co-delivery of TALEN mRNA and AAV donor templates.

FIG. 2G provides additional representative FACS plots showing GFP expression at Day 15. N=3 and represents the number of independent experiments performed using cells from three different donors. FIG. 2H shows results from a test for nuclease specificity utilizing AAV without homology arms. Primary T cells transfected with TALENs and transduced with an AAV vector with an MND promoter driven blue fluorescent protein (BFP) without any homology arms. Fluorescence from this vector at day 15 is indicative of random integration. n=3 and represents the number of independent experiments performed using cells from 3 different donors. Data are presented as mean±SEM. FIG. 2I shows representative FACS plots with GFP expression at day 15 post co-delivery of TALEN mRNA and AAV donor templates. N=3 and represents the number of independent experiments performed using cells from three different donors.

FIGS. 3A-3H depict human primary T cell editing with CRISPR and AAV donors. FIG. 3A shows a schematic of editing of the WAS locus using CRISPR in primary T cells. Schematic illustration of the scAAV guide RNA vector (G), donor template DT (PAM mutated) and AAV vector containing both guide and donor sequences (DTG). PAM site was mutated in both DT and DTG templates to abolish cleavage by the guide Shown is a schematic illustration of the scAAV guide RNA vector (#1189 (G)), donor template (#1201 (PAM mutated)), and AAV vector containing both guide and donor sequences (#1215 (DTG)). Both the PAM mutated donor template and the DTG AAVs have the PAM site mutated to abolish cleavage by guide. FIGS. 3B and 3C provide graphs showing time course of % GFP+ cells (FIG. 3B) and cell viability (FIG. 3C). 10% and 20% represent the % culture volume #1215 AAV added. All other AAVs were added at 10% of culture volume. FIG. 3D provides graphs depicting BFP expression when Cas9, guide and MND.BFP vector with no homology arms were delivered. 20% represent the % of culture volume AAVs were added at. AAVs were added at 10% of culture volume unless otherwise specified. FIG. 3E provides FACS plots showing GFP expression at Day 15. N=3 and represents the number of independent experiments performed using cells from three donors. FIG. 3F Bar graphs showing time course of GFP expression. % HR is reported as % GFP at day 15. n=3 and represents the number of independent experiments performed using cells from 3 different donors. Data are presented as mean±SEM. 20% represent the % culture volume #1215 AAV added. All other AAVs were added at 10% of culture volume. % HR is reported as % GFP at day 15. n=3 and represents the number of independent experiments performed using cells from 3 different donors. Data are presented as mean±SEM. FIG. 3G shows results from a test for nuclease specificity utilizing AAV without homology arms. Bar graphs depict BFP expression when Cas9, guide and MND.BFP vector with no homology arms were delivered. n=3 and represents the number of independent experiments performed using cells from 3 different donors. Data are presented as mean±SEM. FIG. 3H provides representative FACS plots showing GFP expression at day 15.

FIGS. 4A-4D depict the editing mobilized adult CD34⁺ cells using co-delivery of TALEN and AAV. FIG. 4A depicts a timeline of gene editing procedure for human mobilized adult CD34⁺ cells. FIGS. 4B and 4C depicts time course of GFP expression indicative of HDR (FIG. 4B) and viability (FIG. 4C). FIG. 4D depicts representative FACS plots showing GFP expression at Day 5.

FIGS. 5A-5G depict the editing mobilized adult CD34⁺ cells using co-delivery of TALEN mRNA or CRISPR guide delivered as RNP and AAV donor and shows disruption of the WAS locus in human CD34⁺ cells using TALENs or RNP. Mobilized human CD34⁺ cells were cultured in SCGM media supplemented with TPO, SCF, FLT3L (100 ng/mL) and IL3 (60 ng/mL) for 48 hours, followed by electroporation using Neon electroporation system with either 1 ug of each TALEN monomer or Ribonucleoprotein complex (RNP) of Cas9 protein and single guide RNA mixed in 1:1.2 ratio. The sgRNA was purchased from Trilink BioTechnologies and has chemically modified nucleotides at the three terminal positions at 5′ and 3′ ends. The cells were cultured for 5 days and genomic DNA was extracted. The region surrounding the cut site for WAS TALEN and guide was amplified and cloned into pJET cloning vector. Colony sequencing was performed to quantify % cleavage at the cut site by analyzing the indels. N=3 and represents the number of independent experiments performed using cells from three donors. FIG. 5A shows disruption of the WAS locus in human CD34⁺ cells using TALENs or RNP. Mobilized human CD34⁺ cells were cultured in SCGM media supplemented with TPO, SCF, FLT3L (100 ng/mL) and IL3 (60 ng/mL) for 48 hours, followed by electroporation using Neon electroporation system with either 1 ug of each TALEN monomer mRNA or Ribonucleoprotein complex (RNP) of Cas9 protein and single guide RNA mixed in 1:1.2 ratio. The sgRNA was purchased from Trilink BioTechnologies and has chemically modified nucleotides at the three terminal positions at 5′ and 3′ ends. The cells were cultured for 5 days and genomic DNA was extracted. The region surrounding the cut site for WAS TALEN and guide was amplified and cloned into pJET cloning vector. Colony sequencing was performed to quantify % cleavage at the cut site by analyzing the indels. N=3 and represents the number of independent experiments performed using cells from three donors. FIG. 5B shows results from HDR editing of the WAS locus in CD34⁺ HSCs using co-delivery of TALEN mRNA or RNP and AAV donor template. FIG. 5C shows FACS plots depicting GFP expression from Mock, AAV or AAV plus TALEN treated CD34+ cells (top row) or AAV+RNP treated cells (bottom row) 5 days post editing. FIG. 5D shows the cell viability of the edited cells. Bar graphs represent viability of mock and edited cells 2 days post editing. N=8 for RNP, n=12 for TALEN and represents >4 independent donors. FIG. 5E shows colony forming unit (CFU) assay for TALEN edited CD34⁺ cells and FIG. 5F shows the results from a CFU assay for RNP edited CD34⁺ cells. Briefly, 500 cells from edited or untreated (mock) were plated in duplicate in Methocult H4034 media (Stemcell Technologies), incubated at 37′C for 12-14 days and colonies enumerated based on their morphology and GFP expression. CFU-E: Colony forming unit erythroid, M: Macrophage, GM: Granulocyte, macrophage, G: Granulocyte, GEMM: Granulocyte, erythroid, macrophage, megakaryocyte, BFU-E: Burst forming unit erythroid. n=3 experiments and 2 donors. Data are presented as mean±SEM. FIG. 5G shows results from a digital droplet PCR assay or flow cytometry staining for determining HDR.

FIGS. 6A-6D show editing of the WAS locus in CD34⁺ HSCs using co-delivery of TALEN mRNA/RNP and AAV donor template. Adult mobilized human CD34⁺ cells that were cultured in SCGM media as described in FIG. 5, followed by electroporation using Neon electroporation system with either TALEN mRNA or RNP complex. AAV vector carrying the donor template was added immediately after electroporation. Controls included un-manipulated cells (mock) and cells transduced with AAV only without transfection of a nuclease (AAV). FIG. 6A shows Graphs showing % GFP at day 5 indicative of HDR. FIG. 6B shows viability of edited cells 2 days post editing. N=7 for TALEN and N=3 for RNP and represent independent donors. FIGS. 6C and 6D show edited and mock cells that were plated one day post editing onto Methocult media for colony formation unit (CFU) assay. Briefly, 500 cells were plated in duplicate in Methocult H4034 media (Stemcell Technologies, incubated at 37° C. for 12-14 days and colonies enumerated based on their morphology and GFP expression. FIG. 6C shows data from a TALEN experiment and FIG. 6D from an RNP experiment. CFU-E: Colony forming unit erythroid, M: Macrophage, GM: Granulocyte, macrophage, G: Granulocyte, GEMM: Granulocyte, erythroid, macrophage, megakaryocyte, BFU-E: Burst forming unit erythroid.

FIGS. 7A-7C show engraftment of edited human CD34⁺ cells into immune deficient NSG mice. 2 million human CD34⁺ cells untreated, AAV treated or treated with AAV and RNP were injected into immune deficient NSG mice preconditioned with 25 mg/kg busulfan. The mice were sacrificed 10 weeks post-transplant and BM was harvested. FIG. 7A depicts total engraftment of edited cells as defined by expression of human CD45 marker. FIG. 7B illustrates % GFP+ cells within the engrafted cells. FIG. 7C depicts FACS plots from representative mice.

FIG. 8 shows the design of AAV vectors expressing cDNA for human codon optimized WAS gene. AAV vectors with 0.6 kb homology arms flanking either a promoter-less GFP (top) or WAS cDNA (bottom) followed by a shorter WPRE, designated WPRE3 followed by SV40 polyadenylation signal.

FIG. 9A shows data representative of assays for cell viability and % HR in input cells used for engraftment in NSG mice. Adult mobilized human CD34⁺ cells were cultured in SCGM media as previously described with the exception that IL-6 (100 ng/ml) was added instead of IL-3. The cells were electroporated with 1 μg of RNP complex and AAV at an MOI of 62. Data are presented as mean±SEM. N=5 and represents independent experiments using multiple donors.

FIG. 9B shows the data representing the engraftment of edited cells in bone marrow of transplanted NSG mice. Six to 10-week old NSG mice were treated with 25 or 35 mg/kg of BUSULFEX (Henry Schein Inc.) via intraperitoneal injection, diluted 1:1 in phosphate-buffered saline. Twenty-four hours later, 2×10⁶ mock or gene edited hematopoietic stem cells (cultured as described in FIG. 5A) in phosphate-buffered saline were delivered via retro-orbital injection. Animals were euthanized 10 to 16 weeks post-transplant, bone marrow and spleens were harvested and analyzed for human cell engraftment. Dot plot depicts total engraftment of edited cells as defined by expression of human CD45 marker in BM of sacrificed mice. Dots represent individual mice. Data are presented as mean±SEM.

FIG. 9C shows data that represent the engraftment of edited cells in bone marrow of NSG mice 16 weeks post transplantation. Representative flow plots of cells harvested from the bone marrow of NSG mice 16 weeks following transplant. On left, bone marrow harvested from mouse transplanted with untreated cells. On right, bone marrow harvested from mouse transplanted with cells treated with AAV plus RNP. Top row, from left to right: hCD45:mCD45 chimerism, human CD45-gated CD33⁺ and CD19⁺ staining. Bottom row, from left to right: GFP expression among hCD45⁺, CD33⁺ and CD19⁺ cells.

FIG. 9D shows data that represent the engraftment of edited cells in spleens of transplanted mice. Graph depicts total engraftment of edited cells at 10-16 weeks as defined by expression of human CD45 marker in spleens of sacrificed mice. Dots represent individual mice. Data are presented as mean±SEM.

FIG. 9E shows data that represent the engraftment of edited cells in spleens of NSG Mice. Representative flow plots of cells harvested from the spleens of NSG mice 16 weeks following transplant. On left, spleen harvested from mouse transplanted with untreated cells. On right, spleen harvested from mouse transplanted with cells treated with AAV plus RNP. Top row, from left to right: hCD45:mCD45 chimerism, Human CD45-gated CD33⁺ and CD19⁺ staining. Bottom row, from left to right: GFP expression among hCD45⁺, CD33⁺ and CD19⁺ cells. FIG. 9F shows data that represent the engraftment of GFP⁺ cells in NSG mice. HDR-editing rate (% GFP⁺) among hCD45⁺ cells recovered from the bone marrow and spleens of NSG mice sacrificed at 10-16 weeks. Data are presented as mean±SEM.

FIG. 10A shows data that represent the engraftment of edited cells in bone marrow of NSGW41 mice 16 weeks post transplantation. On right, bone marrow harvested from mouse transplanted with cells treated with AAV plus RNP. Top row, from left to right: hCD45:mCD45 chimerism, human CD45-gated CD33⁺ and CD19⁺ staining. Bottom row, from left to right: GFP expression among hCD45⁺, CD33⁺ and CD19′ cells.

FIG. 10B shows data that represent the engraftment of edited cells in spleens of NSGW41 mice 16 weeks post transplantation. On left, spleen harvested from mouse transplanted with untreated cells. On right, spleen harvested from mouse transplanted with cells treated with AAV plus RNP. Top row, from left to right: hCD45:mCD45 chimerism, Human CD45-gated CD33⁺ and CD19⁺ staining. Bottom row, from left to right: GFP expression among hCD45⁺, CD33⁺ and CD19⁺ cells.

FIG. 11A shows data that represent the design of AAV vectors expressing GFP or cDNA for codon optimized WAS gene from endogenous WAS promoter. AAV vectors with 0.6 kb homology arms flanking either a promoter-less GFP (top) or WAS cDNA (bottom) followed by a shorter WPRE, designated WPRE3 followed by SV40 polyadenylation signal are shown.

FIG. 11B shows data that represent the digital droplet PCR to determine editing rates in WAS targeted promoter-less GFP construct. In-out droplet digital PCR was performed as previously described. Droplets were analyzed on the QX200 Droplet Digital PCR System (Bio-Rad) using QuantaSoft software (Bio-Rad). All experiments were performed on female donors. The ddPCR rates were about half of HR rates by FACS suggesting that only one X chromosome was being targeted, n=3 and represents three independent experiments and donors. Data are presented as mean±SEM.

FIG. 11C shows data that represent the digital droplet PCR to determine editing rates with WAS targeted promoter-less coWAS expressing AAV vectors.

### DETAILED DESCRIPTION

In the following detailed description, reference is made to the accompanying drawings, which form a part hereof. In the drawings, similar symbols typically identify similar components, unless context dictates otherwise. The illustrative alternatives described in the detailed description, drawings, and claims are not meant to be limiting. Other alternatives may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, N Y 1989). For purposes of the present disclosure, the following terms are defined below.

The articles “a” and “an” are used herein to refer to one or to more than one (for example, to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight, or length.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process comprises at least the recited steps, but may include additional steps. When used in the context of a compound, composition or device, the term “comprising” means that the compound, composition or device comprises at least the recited features or components, but may also include additional features or components.

As used herein, a “subject” or a “patient” as described herein, have their plain and ordinary meaning when read in light of the specification, and may include but is not limited to, for example, an animal that is the object of treatment, observation or experiment. “Animal” comprises cold- and warm-blooded vertebrates and invertebrates such as fish, shellfish, reptiles and, in particular, mammals. “Mammal” comprises, without limitation, mice, rats, rabbits, guinea pigs, dogs, cats, sheep, goats, cows, horses, primates, such as monkeys, chimpanzees, and apes, and, in particular, humans. In some alternatives, the subject is human.

Some alternatives disclosed herein relate to selecting a subject or patient in need. In some alternatives, a patient is selected who is in need of treatment, amelioration, inhibition, progression, or improvement in disease symptoms or who is in need of curative therapy. In some alternatives, a patient is selected who has symptoms of Wiskott-Aldrich Syndrome (WAS) or X-linked thrombocytopenia (XLT), or who has been diagnosed with WAS or XLT. Such identification or selection of said subjects or patients in need can be made through clinical and/or diagnostic evaluation.

As used herein, the term “treatment” as described herein, has their plain and ordinary meaning when read in light of the specification, and may include but is not limited to, for example, an intervention made in response to a disease, disorder or physiological condition manifested by a subject, particularly a subject suffering from an X-linked disorder, such as WAS or XLT. The aim of treatment may include, but is not limited to, one or more of the alleviation or prevention of symptoms, slowing or stopping the progression or worsening of a disease, disorder, or condition, curative treatment of the disease, disorder, or condition, and the remission of the disease, disorder, or condition. In some alternatives, “treatment” refers to both treatment of the underlying disease or treatment of the disease symptoms. For example, in some alternatives, treatments reduce, alleviate, ameliorate, or eradicate the symptom(s) of the disease and/or provide curative therapy of the disease.

“Adoptive cellular therapy” or “adoptive cell transfer,” as described herein, have their plain and ordinary meaning when read in light of the specification, and may include but is not limited to, for example, transfer of cells, most commonly immune-derived cells, back into the same patient or into a new recipient host with the goal of transferring the immunologic functionality and characteristics into the new host. In some alternatives, adoptive cellular therapy or adoptive cell transfer comprises administering cells for promoting homology directed repair of a WAS gene in a subject.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The terms “nucleic acid” and “polynucleotide” also specifically include nucleic acids composed of bases other than the five biologically occurring bases (adenine, guanine, thymine, cytosine and uracil).

As used herein, the term “fusion” or “fused” as described herein, have their plain and ordinary meaning when read in light of the specification, and may include but is not limited to, for example, a first nucleic acid linked to a second nucleic acid by a phosphodiester bond, so that a coding sequence at the 3′ end of the first nucleic acid is in frame with a coding sequence at the 5′ end of the second nucleic acid, and by extension can further refer to a first polypeptide linked by a peptide bond to a second polypeptide at the C-terminus of the first polypeptide. As such, a “fused” (or “fusion of a”) nucleic acid or peptide as used herein refers to a configuration of molecules, and does not necessarily involve performing the act of joining two molecules together. By way of example, the fusion of a first nucleic acid to a second nucleic acid can encode a single polypeptide in which a first polypeptide sequence (encoded by the first nucleic acid) is fused to a second polypeptide sequence (encoded by the second nucleic acid). In some alternatives, the molecule comprising the fused nucleic acids is referred to as a fusion nucleic acid.

As used herein, the term “vector” as described herein, have their plain and ordinary meaning when read in light of the specification, and may include but is not limited to, for example, a polynucleotide construct, typically a plasmid or a virus, used to transmit genetic material to a host cell. Vectors can be, for example, viruses, plasmids, cosmids, or phage. A vector as used herein can be composed of either DNA or RNA. In some alternatives, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein “AAV system” or “AAV expression system” as described herein, have their plain and ordinary meaning when read in light of the specification, and may include but is not limited to, for example, nucleic acids for expressing at least one transcript-encoding nucleic acid, and which are disposed on one or more AAV vectors. As used herein, “activity-dependent expression” (and variations of this root term) refers to nucleic acid expression that will be induced upon a change in a particular type of activity of a cell containing the nucleic acid, for example depolarization of the cell. In some alternatives, the cell is a neuron, and depolarization of the neuron in response to a stimulus induces “activity-dependent” nucleic acid expression. In some alternatives, an AAV vector includes a sequence as set forth in SEQ ID NOs: 5, 6, 7, 8, 9, 9, 10, 11, 12, 13, 14, 15, 21, 22, 23, or 26.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein “upstream” as described herein, have their plain and ordinary meaning when read in light of the specification, and may include but is not limited to, for example, positions 5′ of a location on a polynucleotide, and positions toward the N-terminus of a location on a polypeptide. As used herein “downstream” refers to positions 3′ of a location on nucleotide, and positions toward the C-terminus of a location on a polypeptide.

The term “construct,” as used herein, as described herein, have their plain and ordinary meaning when read in light of the specification, and may include but is not limited to, for example, a recombinant nucleic acid that has been generated for the purpose of the expression of a specific nucleotide sequence(s), or that is to be used in the construction of other recombinant nucleotide sequences.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature. In some alternatives, the promoter described herein can be a U6 promoter or an MND promoter.

As used herein, the term “enhancer” refers to a type of regulatory element that can modulate the efficiency of transcription, regardless of the distance or orientation of the enhancer relative to the start site of transcription.

As used herein, the term “variant” as described herein, have their plain and ordinary meaning when read in light of the specification, and may include but is not limited to, for example, a polynucleotide (or polypeptide) having a sequence substantially similar to a reference polynucleotide (or polypeptide). In the case of a polynucleotide, a variant can have deletions, substitutions, or additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least 50%, 55%, 60%, 65%, 70%, 75%, 80%, 85%, 90%, 91%, 92%, 93%, 94%, 95%, 96%, 97%, 98%, 99% or more sequence identity to the reference polynucleotide as determined by sequence alignment programs known by skilled artisans, or an amount within a range defined by any two of the aforementioned values. In the case of a polypeptide, a variant can have deletions, substitutions, or additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least 60%, 65%, 70%, 75%, 80%, 85%, 90%, 91%, 92%, 93%, 94%, 95%, 96%, 97%, 98%, 99% or more sequence identity to the reference polypeptide as determined by sequence alignment programs known by skilled artisans, or an amount within a range defined by any two of the aforementioned values.

As used herein, the term “transfection” as described herein, have their plain and ordinary meaning when read in light of the specification, and may include but is not limited to, for example, introduction of a nucleic acid into a host cell, such as by contacting the cell with a recombinant AAV vector as described herein. As used herein, “transient transfection” refers to the introduction of exogenous nucleic acid(s) into a host cell by a method that does not generally result in the integration of the exogenous nucleic into the genome of the transiently transfected host cell. In some alternatives, the nucleic acid is RNA. In some alternatives, the nucleic acid is DNA. In some alternatives, when the nucleic acid is RNA, the nucleic acid does not generally integrate in the genome of the transiently transfected cell. In some alternatives, when the nucleic acid is DNA, the nucleic acid can integrate in the genome of the transiently transfected cell.

By the term “host cell” as described herein, have their plain and ordinary meaning when read in light of the specification, and may include but is not limited to, for example, a cell that is introduced with Cas9-mRNA/AAV-guide RNA according to the present alternatives, as well as, cells that are provided with the systems herein. Host cells can be prokaryotic cells or eukaryotic cells. Examples of prokaryotic host cells include, but are not limited to E. coli, nitrogen fixing bacteria, Staphylococcus aureus, Staphylococcus albus, Lactobacillus acidophilus, Bacillus anthracis, Bacillus subtilis, Bacillus thuringiensis, Clostridium tetani, Clostridium botulinum, Streptococcus mutans, Streptococcus pneumoniae, mycoplasmas, and cyanobacteria. Examples of eukaryotic host cells include, but are not limited to, protozoa, fungi, algae, plant, insect, amphibian, avian and mammalian cells. In some alternatives, a system for editing at least one target gene in a cell is provided, wherein the cell is a eukaryotic cell. In some alternatives, the cell is a mammalian cell. In some alternatives, the cell is a human cell. In some alternatives, the cell is a primary cell. In some alternatives, the cell is not a transformed cell. In some alternatives, the cell is a primary lymphocyte. In some alternatives, the cell is a primary lymphocyte, a CD34⁺ stem cell, a hepatocyte, a cardiomyocyte, a neuron, a glial cell, a muscle cell or an intestinal cell.

“T cell precursors” as described herein, have their plain and ordinary meaning when read in light of the specification, and may include but is not limited to, for example, lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development, they become double-positive thymocytes (CD4⁺CD8⁺), and finally mature to single-positive (CD4⁺CD8⁻ or CD4⁻CD8⁺) thymocytes that are then released from the thymus to peripheral tissues.

“Hematopoietic stem cells” or “HSC” as described herein, have their plain and ordinary meaning when read in light of the specification, and may include but is not limited to, for example, precursor cells that can give rise to myeloid cells such as, for example, macrophages, monocytes, macrophages, neutrophils, basophils, eosinophils, erythrocytes, megakaryocytes/platelets, dendritic cells and lymphoid lineages (such as, for example, T-cells, B-cells, NK-cells). HSCs have a heterogeneous population in which three classes of stem cells exist, which are distinguished by their ratio of lymphoid to myeloid progeny in the blood (L/M). In some alternatives, the cells provided are HSC cells. In some alternatives, the cell is a primary lymphocyte or a CD34⁺ stem cell.

As used herein, “autologous” refers to the donor and recipient of the stem cells being the same, for example, the patient or subject is the source of the cells.

“Primary human cells” as described herein, are directly cultured from their source organ tissue or blood cells. Compared to immortalized cell lines, primary human cells provide enhanced replication of in vivo. In some alternatives, the cells provided are primary human cells.

As used herein, the term “co-delivery” as described herein, have their plain and ordinary meaning when read in light of the specification, and may include but is not limited to, for example, delivery of two or more separate chemical entities, whether in vitro or in vivo. Co-delivery refers to the simultaneous delivery of separate agents; to the simultaneous delivery of a mixture of agents; as well as to the delivery of one agent followed by delivery of a second agent or additional agents. In all cases, agents that are co-delivered are intended to work in conjunction with each other. In some alternatives, for example, co-delivery comprises delivery of an mRNA of interest and an AAV vector.

The term “endonuclease” as described herein, have their plain and ordinary meaning when read in light of the specification, and may include but is not limited to, for example, enzymes that cleave the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” The methods and compositions described herein may be applied to cleavage sites generated by endonucleases. In some alternatives of the system, the system can further provide nucleic acids that encode an endonuclease, including zinc finger nucleases (ZFNs), TAL effector nucleases (TALENs), meganucleases (such as MegaTALs), and CRISPR/Cas9 or a fusion protein comprising a domain of an endonuclease, for example, Cas9, TALEN, or MegaTAL, or one or more portion thereof. These examples are not meant to be limiting and other endonucleases and alternatives of the system and methods comprising other endonucleases and variants and modifications of these exemplary alternatives are possible without undue experimentation.

The term “transcription activator-like (TAL) effector nuclease” (TALEN) as described herein, have their plain and ordinary meaning when read in light of the specification, and may include but is not limited to, for example, a nuclease comprising a TAL-effector domain fused to a nuclease domain. TAL-effector DNA binding domains may be engineered to bind to a desired target and fused to a nuclease domain, such as the Fokl nuclease domain, to derive a TAL effector domain-nuclease fusion protein. The methods and systems described herein may be applied to cleavage sites generated by TAL effector nucleases. In some alternatives of the systems provided herein, the systems can further comprise a TALEN nuclease or a vector or nucleic acid encoding a TALEN nuclease. In some alternatives of the methods provided herein, the method can further comprise providing a nuclease, such as a TALEN nuclease.

CRISPRs (clustered regularly interspaced short palindromic repeats) are segments of prokaryotic DNA containing short repetitions of base sequences. Each repetition is followed by short segments of “spacer DNA” from previous exposures to a bacterial virus or plasmid.

Cas9 (CRISPR associated protein 9) is an RNA-guided DNA endonuclease enzyme associated with the CRISPR adaptive immunity system in Streptococcus pyogenes, among other bacteria. S. pyogenes utilizes Cas9 to memorize and later interrogate and cleave foreign DNA, such as invading bacteriophage DNA or plasmid DNA. Cas9 performs this interrogation by unwinding foreign DNA and checking for complementarity to the 20 base pair spacer region of the guide RNA. If the DNA substrate is complementary to the guide RNA, Cas9 cleaves the invading DNA.

The CRISPR/Cas system as described herein, is used for gene editing (adding, disrupting, or changing the sequence of specific genes) and gene regulation. By delivering the Cas9 protein, a derivative, or fragment thereof and appropriate guide RNAs into a cell, the organism's genome can be cut at any desired location. It can be possible to use CRISPR to build RNA-guided genes capable of altering the genomes of entire populations. The basic components of CRISPR/Cas9 system comprise a target gene, a guide RNA, and a Cas9 endonuclease, derivative, or fragment thereof. An important aspect of applying CRISPR/Cas9 for gene editing is the need for a system to deliver the guide RNAs efficiently to a wide variety of cell types. This could for example involve delivery of an in vitro generated guide RNA as a nucleic acid (the guide RNA generated by in vitro transcription or chemical synthesis). In some alternatives the nucleic acid encoding the guide RNA is rendered nuclease resistant by incorporation of modified bases, such as 2′O-methyl bases. In some alternatives, the CRISPR/Cas9 system described herein, whereby the polynucleotide encoding the Cas9 nuclease or a derivative or functional fragment thereof (for example, a 20 nucleic acid sequence of an mRNA vector with Cas9) is provided with a poly(T) or poly(A) tail of a desired length and prepared in accordance with the teachings described herein, for example, is provided with a guide RNA that comprises one or more modified bases, such as any one or more of the modified bases described herein.

Exemplary guide RNAs useful with the alternatives described herein, which may contain one or more of the modified bases set forth herein. In some alternatives, the modified guide RNA includes the sequences provided in SEQ ID NO: 10. Furthermore, an important system for expressing guide RNAs in this context is based on the use of adeno-associated virus (AAV) vectors because AAV vectors are able to transduce a wide range of primary cells. AAV vectors do not cause infection and are not known to integrate into the genome. Therefore, the use of AAV vectors has the benefits of being both safe and efficacious.

The term “complementary to” means that the complementary sequence is homologous to all or one or more portions of a reference polynucleotide sequence. For illustration, the nucleotide sequence “CAT” corresponds to a reference sequence “CAT” and is complementary to a reference sequence “GTA.”

As used herein, “homology-directed repair” (HDR) as described herein, have their plain and ordinary meaning when read in light of the specification, and may include but is not limited to, for example, DNA repair that takes place in cells, for example, during repair of a double-stranded break (DSB) in DNA. HDR requires nucleotide sequence homology and uses a donor polynucleotide to repair the sequence where the DSB (e.g., within a target DNA sequence) occurred. The donor polynucleotide generally has the requisite sequence homology with the sequence flanking the DSB so that the donor polynucleotide can serve as a suitable template for repair. HDR results in the transfer of genetic information from, for example, the donor polynucleotide to the DNA target sequence. HDR may result in alteration of the DNA target sequence (e.g., insertion, deletion, mutation) if the donor polynucleotide sequence differs from the DNA target sequence and part or all of the donor polynucleotide is incorporated into the DNA target sequence. In some alternatives, an entire donor polynucleotide, a portion of the donor polynucleotide, or a copy of the donor polynucleotide is integrated at the site of the DNA target sequence.

As used herein, a “guide” as described herein, have their plain and ordinary meaning when read in light of the specification, and may include but is not limited to, for example, any polynucleotide that site-specifically guides a nuclease to a target nucleic acid sequence. In some alternatives, a guide comprises RNA, DNA, or combinations of RNA and DNA.

A “genomic region” is a segment of a chromosome in the genome of a host cell that is present on either side of the target nucleic acid sequence site or, alternatively, also comprises a portion of the target site. The homology arms of the donor polynucleotide have sufficient homology to undergo homologous recombination with the corresponding genomic regions. In some alternatives, the homology arms of the donor polynucleotide share significant sequence homology to the genomic region immediately flanking the target site; it is recognized that the homology arms can be designed to have sufficient homology to genomic regions farther from the target site.

As used herein, “non-homologous end joining” (NHEJ) as described herein, have their plain and ordinary meaning when read in light of the specification, and may include but is not limited to, for example, repair of a DSB in DNA by direct ligation of one end of the break to the other end of the break without a requirement for a donor polynucleotide. NHEJ is a DNA repair pathway available to cells to repair DNA without the use of a repair template. NHEJ in the absence of a donor polynucleotide often results in nucleotides being randomly inserted or deleted at the site of the DSB.

As used herein “cleavage site” refers to a sequence that mediates the separation of a first polypeptide that would otherwise be in cis to a second polypeptide. Accordingly, for simplicity, “cleavage,” “cleavage site,” and the like as used herein refer to the separation of any two polypeptides that are encoded by a single polynucleotide in cis. Thus, “cleavage” and “cleavage site,” can, but do not necessarily refer to proteolytic sites and events, and can also refer to other mechanisms for mediating the separation of polypeptides, for example ribosomal skipping.

As used herein, the term “label” refers to a detectable molecule. A number of suitable labels comprise polypeptides. As such, as used herein, a “label nucleic acid” refers to a nucleic acid encoding a label. In some alternatives, the AAV vector systems comprise a label polynucleotide. Thus, in some alternatives, a promoter (such as an MND promoter) is operatively linked to a label polynucleotide, such that the AAV vectors described herein comprise a reporter. Example labels that are suitable in accordance with alternatives herein include, but are not limited to, green fluorescent protein (GFP), including, for example, Aequoria victoria GFP, Renilla muelleri GFP, Renilla remformis GFP, Renilla ptilosarcus, blue fluorescent protein (BFP), yellow fluorescent protein (YFP), red fluorescent protein (RFP), cyan fluorescent protein (CFP), or orange fluorescent proteins (OFP). Additional reporter genes include, but are not limited to neomycin, phosphoro-transferase, chloramphenicol acetyl transferase, thymidine kinase, luciferase, β-glucuronidase, aminoglycoside, phosphotransferase, hygromycin B, xanthine-guanine phosphoribosyl, luciferases (e.g., renilla, firefly, etc.), DHFR/methotrexate, β-galactosidase, alkaline phosphatase, turbo and tagRFP, and nuclear targeted versions of any of the aforementioned reporter genes. In some alternatives, the polypeptide of interest comprises the label itself, for example when production of label in active cells is desired. In some alternatives, an AAV construct provided herein comprises a U6 promoter driven guide RNA cassette or an MND promoter driven GFP cassette, or both, and wherein the MND promoter driven GFP cassette provides for tracking of AAV transduction efficiency.

The term “gene expression” as described herein, have their plain and ordinary meaning when read in light of the specification, and may include but is not limited to, for example, biosynthesis of a gene product. For example, in the case of a structural gene, gene expression involves transcription of the structural gene into mRNA and the translation of mRNA into one or more polypeptides.

A “polypeptide” is a polymer of amino acid residues joined by peptide bonds, whether produced naturally or synthetically. Polypeptides of less than 10 amino acid residues are commonly referred to as “peptides.” A polypeptide can be considered as a protein.

A “protein” is a macromolecule comprising one or more polypeptide chains. A protein may also comprise non-peptide components, such as carbohydrate groups. Carbohydrates and other non-peptide substituents may be added to a protein by the cell in which the protein is produced, and will vary with the type of cell. Proteins are defined herein in terms of their amino acid backbone structures; substituents such as carbohydrate groups are generally not specified, but may be present nonetheless. In some alternatives, a system for editing at least one target gene in a cell is provided, wherein the method comprises a first nucleic acid sequence encoding a CRISPR guide RNA, wherein the CRISPR guide RNA is complimentary to at least one target gene in a cell and, wherein said first nucleic acid sequence is present in a vector; said system also comprising a second nucleic acid sequence encoding a Cas9 protein, a third nucleic acid sequence encoding a first adenoviral protein and a fourth nucleic acid sequence encoding a second adenoviral protein. In some alternatives, the CRISPR guide sequence is defined by SEQ ID NO: 3, 5, 6, 7 and 8. In some alternatives, the CRISPR guide sequence is defined by SEQ ID NO: 31, 32, 33, 34.

### X-Linked Disorders

Some alternatives provided herein relate to treating, ameliorating, inhibiting, or improving an X-linked disorder. In some alternatives, the disorder is Wiskott-Aldrich Syndrome (WAS). WAS is a severe X-linked genetic disorder with an incidence of around 1 in 250,000 live male births. WAS is a result of mutation in the WAS gene. WAS is characterized by opportunistic viral and bacterial infections due to abnormal lymphocyte function, thrombocytopenia with small platelets, eczema, and increased risk of autoimmune disorders and malignancies. In some alternatives, the disorder is X-linked thrombocytopenia (XLT). XLT also results from mutations in the WAS gene leading primarily to thrombocytopenia and bleeding.

Hematopoietic cell transplantation (HCT) is the only widely available approach that can provide curative therapy for WAS. Overall, transplant survival has increased over the last two decades, with improvements in transplant care, donor matching, and earlier treatment. However, outcomes for patients without a human leukocyte antigen (HLA) matched donor, particularly older subjects, remain suboptimal. In addition, patients with mixed chimerism exhibit an increased risk of autoimmunity and low myeloid chimerism predicts persistent thrombocytopenia. Further, optimal conditioning regiments that limit HCT-related morbidity, as well as the late effects following conditions remain unclear.

The challenge of finding suitable donors and other significant complications associated with HCT have prompted efforts to develop alternative gene therapy (GT) strategies for WAS. Pioneering GT trials using integrating viral vectors, based on use of gamma-retroviral (RV) or self-inactivating lentiviral vectors (LV) have been carried out in Europe. Boztug et al. first reported on two patients treated by RV GT. Boztug et al. N Engl J Med, 2010, 363(20), 1918-1927; incorporated by reference herein in its entirety. Both patients showed a reduced frequency and severity of infections and disappearance of bleeding episodes and autoimmune manifestations. In a subsequent follow on trial using hematopoietic stem cell gene therapy, a total of 10 patients were treated. Nine patients showed successful immune reconstitution with platelet counts greater than 80,0000 cells/μL in eight of nine subjects. Braun et al. Sci Transl Med, 2014, 12(6), 227; incorporated by reference herein in its entirety. Strikingly, severe adverse events with insertional mutagenesis leading to leukemia occurred in seven patients.

Based on these adverse events, self-inactivating LV (SIN-LV) encoding WAS cDNA under the control of the native proximal WAS gene promoter were developed. Trials using this LV have opened in Europe and in the United States. Aiuti et al. reported on initial results in three patients treated in Italy. Aiuti et al. Science, 2013, 341, 6148; incorporated by reference herein in its entirety. Long-term engraftment with gene-modified cells was observed in all three patients in the range of 25-50% of the total peripheral blood cells. WASp expression was detected in a vector containing hematopoietic cells with approximate endogenous expression levels in T cells. A subset of immunological abnormalities was corrected. Thrombocytopenia improved, but unlike the results of the WAS RV trial, platelet counts were not corrected. In contrast to the RV trial, clonal analysis indicated no evidence of clonal expansion for greater than three years.

A French/UK group published the outcome of seven WAS patients who underwent GT using the identical LV. Abina et al. JAMA, 2015, 313, 1550-1563; incorporated by reference herein in its entirety. However, similar to data from Aiuti et al., thrombocytopenia improved but platelet counts were not corrected. In summary, to date WAS LV trials have shown sustained viral marking and WASp expression in multiple hematopoietic lineages with partial clinical improvement. However, LV therapy has failed to rescue thrombocytopenia. Further, the long-term response to immunization and infections challenge and risk for autoimmunity remain to be determined.

Thus, provided herein are systems and methods for the introduction of an intact WAS cDNA under control of the endogenous promoter and enhancer in hematopoietic stem cells (HSCs). In some alternatives, the systems and methods described herein rescue immunologic and functional defects in WAS and XLT and provide a curative therapy. As discussed above, WAS gene replacement using viral-based gene therapy has been used to partially correct this disease, current viral vector approaches have led to increased risk for malignancy (when using gamma-retroviral vectors) or have failed to correct all hematopoietic deficits, including, most notably, the failure to correct thrombocytopenia. However, the systems and methods provided herein for gene editing of the endogenous WAS gene lead to improved long-term disease correction.

Accordingly, the systems and methods described herein provide several unique opportunities for treatment of WAS and XLT. For example, some alternatives described herein relate to editing a gene product, for example, at the WAS locus. In some alternatives, the edited gene product remains under control of the endogenous promoter and enhancer elements. In some alternatives, the editing rates are performed at high efficiency in a cell, such as a hematopoietic stem cell. Accordingly, provided herein are systems and methods for achieving high efficiency editing. Also provided are systems and methods for engrafting edited cells. In some alternatives, the methods and systems provided herein include use of autologous stem cells. In comparison to prior treatments that use marrow transplantation of stem cells from an allogeneic donor, WAS gene editing as described herein uses autologous stem cells. Thus, there is no risk for graft versus host disease, a major problem and risk with transplants. In some alternatives, the methods and systems provided herein use the endogenous gene promoter/enhancer to control gene expression. In comparison to viral gene replacement therapy as used in previous treatments, the gene editing systems and methods described herein use an endogenous gene promoter/enhancer to control gene expression, which leads to endogenous levels of WASp in all relevant cell lineages. Gene editing also eliminates the risk for viral vector mutagenesis that is present in all gene replacement strategies and was observed as a serious adverse event in nearly all WAS patients treated with gamma-retroviral vectors. Gene editing also leads to more appropriate levels of WAS protein expression in comparison to current LV vector therapy. The published trials in WAS LV therapy utilized a vector containing the WAS minimal promoter, which partially rescued function in some cell types but did not rescue platelet production most likely because the minimal promoter was not sufficiently robust in platelet progenitor cells.

Furthermore, in all current therapies for WAS (transplant and LV gene therapy) conditioning regimens are being used. Conditioning is also required for successful application of gene editing in order to achieve a clinically relevant level of engraftment of edited HSC. In some alternatives, engraftment of 10%, 15%, 20%, 25%, 30%, 35%, 40%, 45%, 50%, 55%, 60%, 65% 70%, or 70% of edited cells or an amount within a range defined by any two of the aforementioned values will lead to clinical benefit. In some alternatives, treosulfan/fludarabine conditioning is used.

Some alternatives provided herein relate to unique nuclease reagents based upon either a TALEN or CRISPR/Cas9 platform in parallel with AAV based delivery of novel HDR repair templates. In some alternatives, the combination of a nuclease and an AAV vector achieves high rates of HDR at the WAS locus in human T cells and CD34⁺ HSCs, leading to sustained gene expression in vitro and in vivo following engraftment in immunodeficient mice. In some alternatives, the methods and systems provided herein permit introduction of corrective cDNAs into the WAS locus in autologous cells from control subjects of subjects with WAS or XLT, thereby permitting lineage and developmental specific regulation of the functional gene product in vitro and in vivo. In some alternatives, the methods and systems provided herein permit long-term sustained cell lineage appropriate therapeutic WASp expression without adverse events within the host genome.

In some alternatives, the systems and methods described herein minimize the risk of mutagenesis by targeting integration of a therapeutic cassette into the WAS locus. In some alternatives, the systems described herein include an AAV donor template for integrating an expression cassette into the first exon of WAS by homology directed repair. Also provided herein are nuclease platforms targeting the WAS locus, including TALENs and CRISPR/Cas9.

Some alternatives concern methods for using adult mobilized CD34⁺ cells and co-delivery of either TALEN mRNA or Cas9/gRNA ribonucleoprotein complexes (RNPs) and an AAV donor for targeted integration of a promoter-driven fluorescent marker. In some alternatives, the methods provided herein achieve efficient homology directed repair rates across multiple donors at an efficiency of 10%, 15%, 20%, 25%, 30%, 35%, 40%, 45%, 50%, 60%, 70%, 80%, or greater, or an efficiency within a range defined by any two of the aforementioned values for TALEN and an efficiency of 10%, 15%, 20%, 25%, 30%, 35%, 40%, 45%, 50%, 60%, 70%, 80%, or greater, or an efficiency within a range defined by any two of the aforementioned values for RNP. In some alternatives, the highest levels of cell viability is observed using RNP/AAV co-delivery. In some alternatives, edited HSC retain their potential to give rise to multiple lineages in colony forming unit assays. In some alternatives, the systems provided herein provide long-term engraftment and differentiation potential in immune-deficient mice. In some alternatives, AAV vectors carrying WAS cDNA restore expression in WAS deficient cells. In some alternatives, the systems and methods described herein provide therapeutic correction of the disease or a disease symptom in patients.

### Homology Directed Repair

Homology directed repair (HDR), refers to the process of repairing DNA damage using a homologous nucleic acid (e.g., a sister chromatid or an exogenous nucleic acid). In a normal cell, HDR typically involves a series of steps such as recognition of the break, stabilization of the break, resection, stabilization of single stranded DNA, formation of a DNA crossover intermediate, resolution of the crossover intermediate, and ligation. As described herein, HDR can be used to alter a target sequence and correct (e.g., repair or edit) a mutation in the genome. While not wishing to be bound by theory, it is believed that alteration of the target sequence occurs by HDR with a donor template or template nucleic acid. For example, the donor template or the template nucleic acid provides for alteration of the target position.

Some alternatives provided herein relate to methods and systems for homology directed repair of the gene associated with a disorder. In some alternatives, the gene is a WAS gene. In some alternatives, the method comprises HDR of the WAS gene in human hematopoietic cells. In some alternatives, the disorder is an X-linked disorder. In some alternatives, the disorder is WAS. In some alternatives, the disorder is XLT. In some alternatives, the method and systems include nuclease-based HDR of the WAS gene. In some alternatives, the nuclease based HDR comprises a TALEN based nuclease. In some alternatives, the nuclease based HDR comprises a CRISPR/Cas based nuclease.

### TALEN

Transcription activator-like (TAL) effector-DNA modifying enzyme (TALEN) is a restriction enzyme that can be engineered to cut specific sequences of DNA. TALENs are made by fusing a TAL-effector domain to a DNA cleavage domain.

In some alternatives, the WAS locus used in targeting with WAS TALENs is co-delivered with an AAV donor.

In some alternatives the WAS TALEN forward sequence is defined by SEQ ID NO: 27 or SEQ ID NO: 29. In some alternatives, the WAS TALEN reverse sequence is defined by SEQ ID NO: 28 or SEQ ID NO: 30. In some alternatives, the AAV donor comprises a GFP cassette under control of an MND promoter. In some alternatives, the AAV donor has a 1 kb homology arm flanking an MND promoter driven GFP cassette (SEQ ID NO: 35). In some alternatives, the AAV donor comprises one or more nucleotide mutations to abolish cleavage by TALENs. In some alternatives, the nucleotide mutation is a mutation of the T preceding the TALEN binding site (SEQ ID NO: 36). In some alternatives, the AAV donor comprises deletion of the entire region between exon 1 up to the reverse TALEN binding site (SEQ ID NO: 37).

Some alternatives provided herein relate to a TALEN nuclease for use in HDR of a gene of interest. In some alternatives, the TALEN binds to a TALEN binding site in a gene of interest. In some alternatives, the gene of interest is a WAS gene (SEQ ID NO: 4). In some alternatives, a WAS TALEN binds to native WAS sequence (SEQ ID NO: 4). The WAS locus used in targeting with WAS TALENs comprises the following components from 5′ to 3′: upstream homology arm (SEQ ID NO: 11); exon 1, including a guide RNA (SEQ ID NO: 19); T-for (TALEN forward binding site; SEQ ID NO: 15); cleavage site (SEQ ID NO: 17); T-rev (TALEN reverse binding site; SEQ ID NO: 16); exon 2 (SEQ ID NO: 18); and downstream homology arm (SEQ ID NOs: 12, 13, or 14).

In some alternatives, the WAS locus used in targeting with WAS TALENs is co-delivered with an AAV donor. In some alternatives the WAS TALEN forward sequence is defined by SEQ ID NO: 1 or SEQ ID NO: 24. In some alternatives, the WAS TALEN reverse sequence is defined by SEQ ID NO: 2 or SEQ ID NO: 25. In some alternatives, the AAV donor comprises a GFP cassette under control of an MND promoter. In some alternatives, the AAV donor has a 1 kb homology arm flanking an MND promoter driven GFP cassette (SEQ ID NO: 5). In some alternatives, the AAV donor comprises one or more nucleotide mutations to abolish cleavage by TALENs. In some alternatives, the nucleotide mutation is a mutation of the T preceding the TALEN binding site (SEQ ID NO: 6). In some alternatives, the AAV donor comprises deletion of the entire region between exon 1 up to the reverse TALEN binding site (SEQ ID NO: 7).

In some alternatives the WAS TALEN forward sequence is defined by SEQ ID NO: 27 or SEQ ID NO: 29. In some alternatives, the WAS TALEN reverse sequence is defined by SEQ ID NO: 28 or SEQ ID NO: 30. In some alternatives, the AAV donor comprises a GFP cassette under control of an MND promoter. In some alternatives, the AAV donor has a 1 kb homology arm flanking an MND promoter driven GFP cassette (SEQ ID NO: 35). In some alternatives, the AAV donor comprises one or more nucleotide mutations to abolish cleavage by TALENs. In some alternatives, the nucleotide mutation is a mutation of the T preceding the TALEN binding site (SEQ ID NO: 36). In some alternatives, the AAV donor comprises deletion of the entire region between exon 1 up to the reverse TALEN binding site (SEQ ID NO: 37).

### CRISPR/Cas

Some alternatives provided herein relate to a Cas nuclease for use in HDR of a gene of interest. In some alternatives, the Cas nuclease is a Cas9 nuclease. Cas9 is an RNA-guided DNA endonuclease enzyme associated with the CRISPR (Clustered Regularly Interspersed Palindromic Repeats) adaptive immunity system in Streptococcus pyogenes, among other bacteria. S. pyogenes utilizes Cas9 to memorize and later interrogate and cleave foreign DNA, such as invading bacteriophage DNA or plasmid DNA. Cas9 performs this interrogation by unwinding foreign DNA and checking for if it is complementary to the 20 base pair spacer region of the guide RNA. If the DNA substrate is complementary to the guide RNA, Cas9 cleaves the invading DNA.

In some alternatives, the Cas nuclease is delivered in a complex with a single guide RNA as a ribonucleoprotein complex (RNP). In some alternatives, the CRISPR guide sequence is defined by SEQ ID NO: 3. In some alternatives, the RNP is co-delivered with an AAV donor. In some alternatives, the AAV donor is a self-complementary AAV (scAAV). In some alternatives, the AAV donor comprises a GFP cassette under control of an MND promoter wherein a protospacer adjacent motif (PAM) site is deleted (SEQ ID NO: 5). In some alternatives, the AAV donor comprises a U6 promoter driven guide RNA cassette (SEQ ID NO: 8). In some alternatives, the AAV donor comprises both the donor and guide sequences (SEQ ID NO: 9). In some alternatives, the AAV donor is an scAAV done including guide sequences, and includes a sequence defined by SEQ ID NO: 21, SEQ ID NO: 22, or SEQ ID NO: 23.

In some alternatives, the Cas nuclease is delivered in a complex with a single guide RNA as a ribonucleoprotein complex (RNP). In some alternatives, the CRISPR guide sequence is defined by SEQ ID NO: 31, 32, 33, 34. In some alternatives, the RNP is co-delivered with an AAV donor. In some alternatives, the AAV donor is a self-complementary AAV (scAAV). In some alternatives, the AAV donor comprises a GFP cassette under control of an MND promoter wherein a protospacer adjacent motif (PAM) site is deleted (SEQ ID NO: 9). In some alternatives, the AAV donor comprises a U6 promoter driven guide RNA cassette (SEQ ID NO: 38). In some alternatives, the AAV donor comprises both the donor and guide sequences (SEQ ID NO: 39).

### Cells

Some alternatives provided herein relate to co-delivery of a nuclease, such as a TALEN or Cas nuclease, and an AAV donor template to modify endogenous WAS locus in a cell. In some alternatives, the cell is a mammalian cell. In some alternatives, the cell is a human cell. In some alternatives, the cell is an autologous cell. In some alternatives, the cell is a primary cell. In some alternatives, the cell is a lymphocyte. In some alternatives, the cell is not a transformed cell. In some alternatives, the cell is a primary lymphocyte. In some alternatives, the cell is a lymphocyte precursor cell. In some alternatives, the cell is a T cell. In some alternatives, the cell is a hematopoietic cell. In some alternatives, the cell is a CD34⁺ cell. In some alternatives, the cell is a primary human hematopoietic cell.

In some alternatives, the cell is transformed by co-delivery of a nuclease, such as a TALEN nuclease or Cas nuclease, and an AAV donor template to modify endogenous WAS locus in a cell. In some alternatives, a method of editing a WAS gene in a cell is provided, wherein the method comprises introducing into a cell a first vector that comprises a first nucleic acid sequence encoding a guide RNA, such as a TALEN guide RNA or a CRISPR guide RNA, wherein the guide RNA is complimentary to at least one target gene in said cell, and introducing into said cell a second nucleic acid sequence encoding a nuclease, such as a TALEN nuclease or a Cas nuclease, a derivative, or fragment thereof. In some alternatives, a cell is provided, wherein the cell is manufactured by the said methods.

### Methods of Treating, Inhibiting, or Ameliorating WAS or XLT in a Subject in Need

Some alternatives provided herein related to methods of promoting HDR of a WAS gene in a subject in need thereof. In some alternatives, the method comprises selecting or identifying a subject in need thereof. A selected or identified subject in need thereof is a subject that presents with symptoms of an X-linked disorder, such as WAS or XLT, or a subject that has been diagnosed with an X-linked disorder, such WAS or XLT. Such evaluations can be made clinically or by diagnostic test.

In some alternatives, the method comprises adoptive cellular therapy or adoptive cell transfer of treated cells to a subject in need. In some alternatives, adoptive cellular therapy or adoptive cell transfer comprises administering cells for promoting homology directed repair of a WAS gene in a subject. In some alternatives, the method comprises obtaining cells from the subject in need thereof. In some alternatives, the cells from the subject in need are primary human hematopoietic cells. In some alternatives, the cells are transformed by co-delivery of a nuclease, such as a TALEN nuclease or a Cas nuclease, and an AAV donor, which modifies the endogenous WAS locus in the cell. In some alternatives, the method comprises expanding the transformed cells. In some alternatives, the method comprises selecting transformed cells that have successful modification of the WAS locus in the cell. In some alternatives, the transformed cells are administered to the patient.

In some alternatives, administration of the transformed cells to the patient comprises administration of autologous cells to the patient. In some alternatives, administration of the transformed cells to the patient treats, inhibits, or ameliorates symptoms of WAS and/or XLT. In some alternatives, administration of the transformed cells to the patient treats WAS and/or XLT. In some alternatives, the method improves thrombocytopenia. In some alternatives, the method increases platelet counts.

In some alternatives, an amount of treated cells is administered to the composition. In some alternatives, the amount of cells administered is 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, or 1×10⁹ cells, or greater, or an amount within a range defined by any two of the aforementioned values.

In some alternatives, the treated cells are administered to a subject as a co-therapy with an additional therapy that is used to treat the symptoms of the disorder or used to treat the disorder. In some alternatives, the additional therapy includes immunoglobulin therapy, an antibiotic therapy, corticosteroid therapy, or transfusion therapy.

### Pharmaceutical Compositions and Administration

Cells prepared by the systems or methods provided herein can be administered directly to a patient for targeted homology directed repair of a WAS locus and for therapeutic or prophylactic applications, for example, for treating, inhibiting, or ameliorating an X-linked disorder, such as WAS or XLT. In some alternatives, cells are prepared by the systems provided herein. In some alternatives, a composition is provided, wherein the composition comprises the cell. In some alternatives, the compositions described herein, can be used in methods of treating, preventing, ameliorating, or inhibiting an X-linked disorder, such as WAS or XLT or ameliorating a disease condition or symptom associated with an X-linked disorder, such as WAS or XLT.

The compositions comprising the cells are administered in any suitable manner, and in some alternatives with pharmaceutically acceptable carriers. Suitable methods of administering such compositions comprising the cells are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as, by the particular method used to administer the composition. Accordingly, there is a wide variety of suitable formulations of pharmaceutical compositions that are available (see, e.g., Remington's Pharmaceutical Sciences).

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

In some alternatives, one or more of parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal routes of administration are contemplated. In some alternatives, the composition to be administered can be formulated for delivery via one or more of the above noted routes.

### ADDITIONAL ALTERNATIVES

Additional alternatives are disclosed in further detail in the following examples, which are not in any way intended to limit the scope of the claims. The examples provided below demonstrate genome editing tools that possess a high degree of specificity, providing the foundation for site-specific modification of the WAS locus for therapeutics.

### Alternative 1

### Cleavage Efficiency of TALENs in Primary T Cells

This alternative demonstrates methods for determining the cleavage efficiency of the systems described herein.

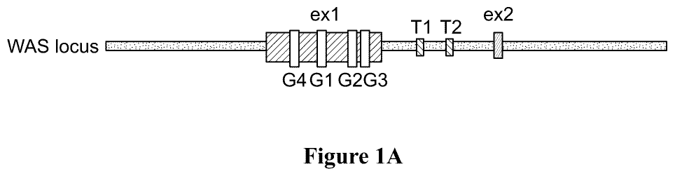

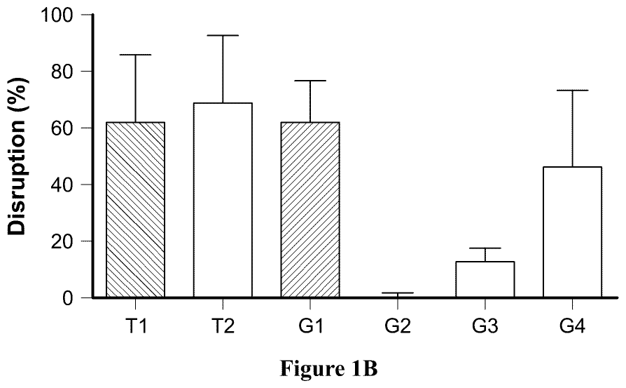

A panel of candidate guide RNAs (delivered as self-complementary AAV (scAAV) and Cas9 as mRNA) or TALEN mRNAs were prepared and tested in T cells. FIG. 1A depicts the location of the WAS TALENs (TALEN #1 and TALEN #2) and guide RNAs, termed G1, G2, G3, and G4 within the human WAS gene.

Primary human T cells were cultured in T cell growth medium supplemented with IL-2 (50 ng/mL), IL-7 (5 ng/mL), and IL-15 (5 ng/mL) and stimulated using CD3/CD28 beads (Dynabeads, Life Technologies) for 48 hours. Beads were removed and cells rested overnight followed by electroporation using Neon Transfection system with either TALEN mRNA (1 μg of each RNA monomer) or co-delivery of 1 μg of Cas9 mRNA and scAAV carrying guide RNA. Cells were cultured for 5 more days and genomic DNA was extracted. The region surrounding the cut site was amplified and purified using PCR purification kit. 200 ng of purified PCR product was incubated with T7 endonuclease (NEB), analyzed on a gel and percent disruption quantified using Licor Image Studio Lite software.

FIG. 1B depicts the percentage of non-homologous end joining (NHEJ) events in primary human T cells detected using the T7 assay, 5 days post-transfection with TALEN mRNAs (T1, T2) or with Cas9 mRNA co-delivered with scAAV expressing gRNAs (G1-4). n=2, represents the number of independent experiments. TALEN T1 and Guide G1 were used in the following examples.

Primary human T cells were cultured in T cell growth medium supplemented with IL-2 (50 ng/ml), IL-7 (5 ng/ml), and IL-15 (5 ng/ml) and stimulated using CD3/CD28 beads (Dynabeads, Life Technologies) for 48 hours. Beads were removed and cells rested overnight followed by electroporation using Neon Transfection system with either TALEN mRNA (1 μg of each RNA monomer) or co-delivery of 1 μg of Cas9 mRNA and scAAV carrying guide RNA. Cells were cultured for 5 more days and the genomic DNA was extracted. The region surrounding the cut site was amplified and purified using PCR purification kit. 200 ng of purified PCR product was incubated with T7 endonuclease (NEB), analyzed on a gel and percent disruption quantified using Licor Image Studio Lite software. TALEN T1 and Guide G1 were used in experiments in subsequent figures.

### Alternative 2

### Editing of Human Primary T Cells with TALEN and AAV Co-Delivery

This alternative demonstrates methods for editing primary T Cells with TALEN and AAV as described in some alternatives herein.

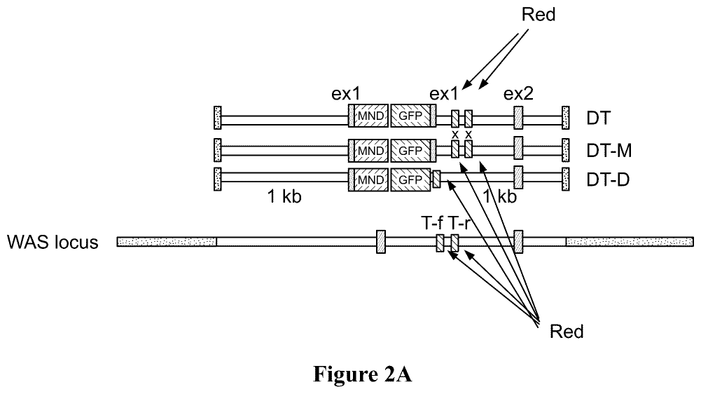

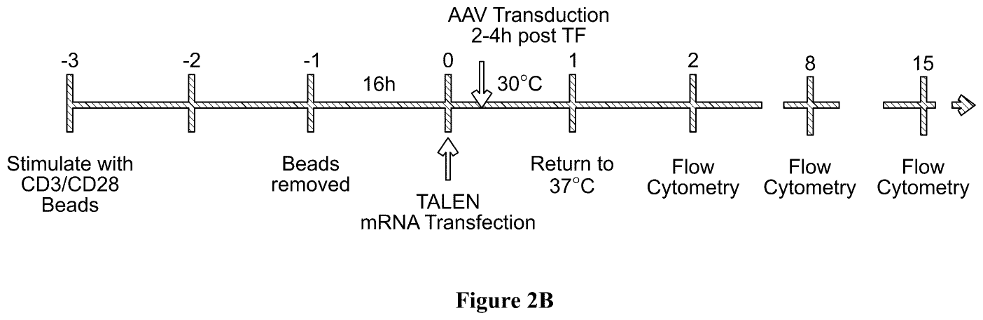

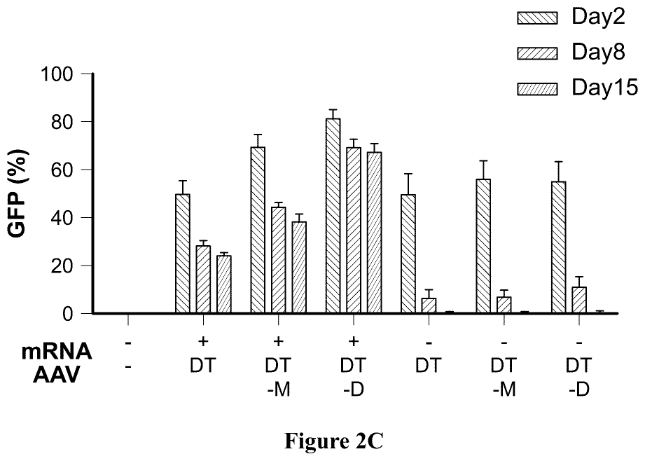

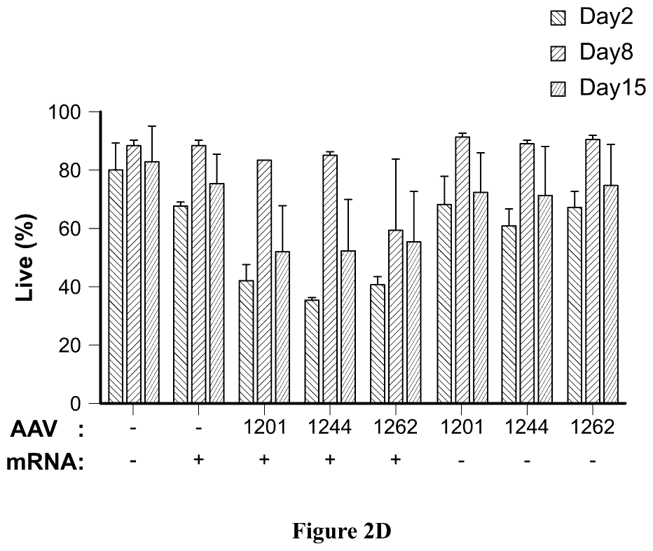

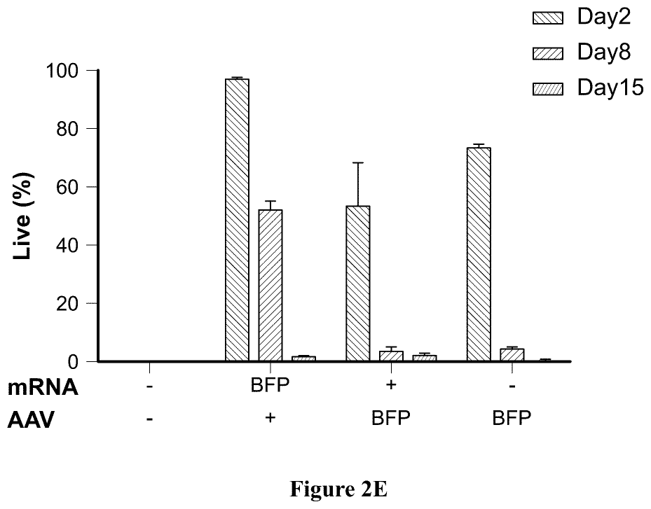

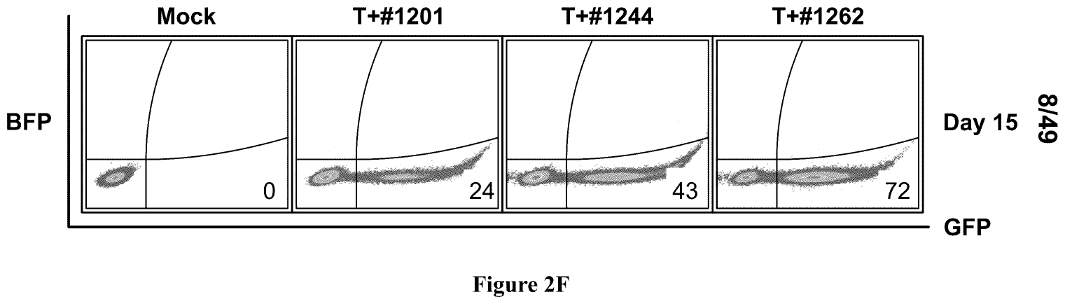

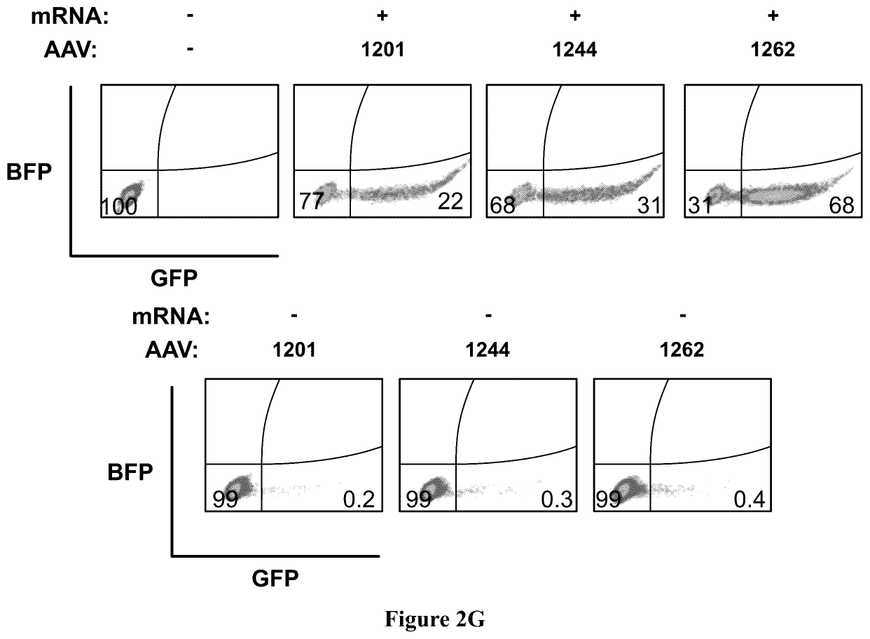

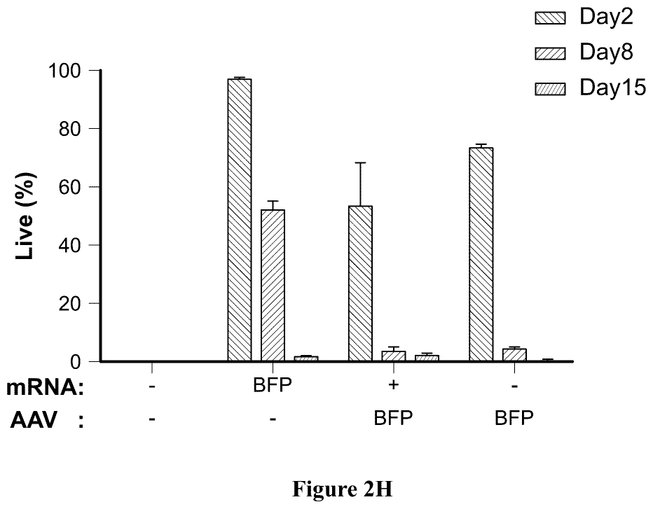

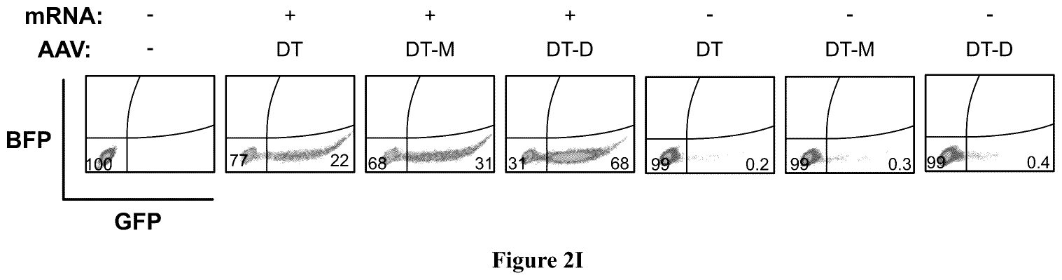

A WAS gene was prepared using the TALEN T1 and Guide G1 as described in Example 1, and as shown in FIG. 2A. FIG. 2A depicts the WAS locus and AAV donor templates used in targeting with WAS TALENs. The WAS locus shows the TALEN binding sites, represented by T-f (TALEN forward) and T-r (TALEN reverse). Homology arms are also shown. The three AAV vectors are represented as vectors #1201, #1244, and #1262. AAV vector #1201 has 1 kb of homology arms flanking an MND promoter driven green fluorescent protein (GFP) cassette. AAV vector #1244 has couple nucleotide mutations (represented by X) to abolish cleavage by TALENs. AAV vector #1262 has the entire region between the exon 1 up to the reverse TALEN binding site deleted.

As shown in FIG. 2A small red boxes represent TALEN forward (T-for) and reverse (T-rev) binding sites. Homology arms are depicted in white. DT AAV vector has 1 kb of homology arms flanking an MND promoter driven green fluorescent protein (GFP) cassette. DT-M AAV donor has couple nucleotide mutations (represented by X) to abolish cleavage by TALENs. The #DT-D vector has the entire region between the exon 1 up to the reverse TALEN binding site deleted.

FIG. 2B depicts the timescale for the experimental method. Primary human T cells were cultured in T cell growth medium supplemented with IL-2 (50 ng/mL), IL-7 (5 ng/mL), and IL-15 (5 ng/mL) and stimulated using CD3/CD28 beads (Dynabeads, Life Technologies) for 48 hours. Primary human T cells were cultured in T cell growth medium supplemented with IL-2 (50 ng/ml), IL-7 (5 ng/ml), and IL-15 (5 ng/ml) and stimulated using CD3/CD28 beads (Dynabeads, Life Technologies) for 48 hours. Beads were removed and cells rested overnight followed by electroporation with the nuclease TALEN mRNA using Neon Transfection system. AAV donors were added 2-4 hrs. post transfection at 20% of culture volume. Cells were analyzed for GFP expression on Days 2, 8 and 15. Day 15 GFP is indicative of homology directed repair (HDR). FIG. 2C depicts the % GFP at days 2, 8, and 15 at the indicated conditions (+/−mRNA and the various AAV vectors). Primary human CD3+ T cells were cultured and bead stimulated. Cells were then transfected with TALEN mRNA and AAV donors added two hours later at 20% of culture volume. Cells were analyzed for GFP expression on Days 2, 8 and 15. GFP expression at day 15 is indicative of homology directed repair (HDR). n=3 and represents the number of independent experiments performed using cells from 3 different donors. Data are presented as mean±SEM. GFP expression at day 15 is indicative of homology directed repair (HDR). n=3 and represents the number of independent experiments performed using cells from 3 different donors. Data are presented as mean±SEM. FIG. 2D shows the cell viability under each condition. FIG. 2E are the results from a test for nuclease specificity utilizing AAV without homology arms. Primary T cells transfected with TALENs and transduced with an AAV vector with an MND promoter driven blue fluorescent protein (BFP) without any homology arms. Fluorescence from this vector at day 15 is indicative of random integration. n=3 and represents the number of independent experiments performed using cells from 3 different donors. Data are presented as mean±SEM. FIG. 2F shows representative FACS plots with GFP expression at day 15 post co-delivery of TALEN mRNA and AAV donor templates. FIG. 2G provides additional representative FACS plots showing GFP expression at Day 15. N=3 and represents the number of independent experiments performed using cells from three different donors. Shown in FIG. 2H are the results from a test for nuclease specificity utilizing AAV without homology arms. Primary T cells transfected with TALENs and transduced with an AAV vector with an MND promoter driven blue fluorescent protein (BFP) without any homology arms. Fluorescence from this vector at day 15 is indicative of random integration. n=3 and represents the number of independent experiments performed using cells from 3 different donors. Data are presented as mean±SEM. FIG. 2I shows representative FACS plots showing GFP expression at day 15. n=3 and represents the number of independent experiments performed using cells from 3 different donors.

### Alternative 3

### Editing of Human Primary T Cells with CRISPR and AAV Co-Delivery

This alternative demonstrates methods for editing primary T Cells with CRISPR and AAV as described in some alternatives herein.

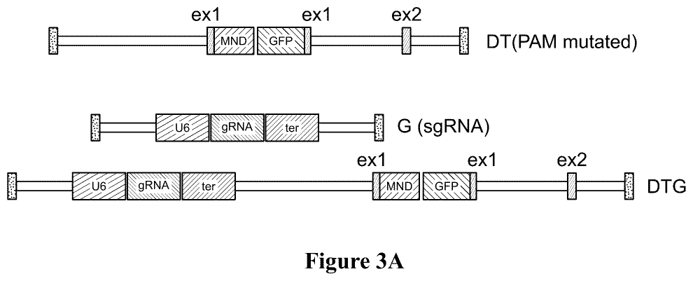

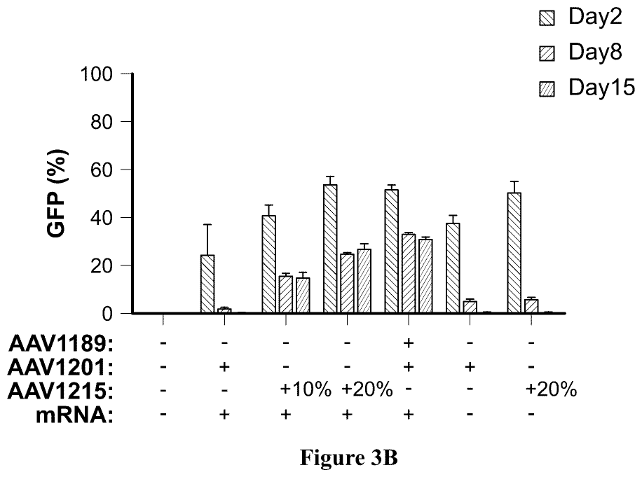

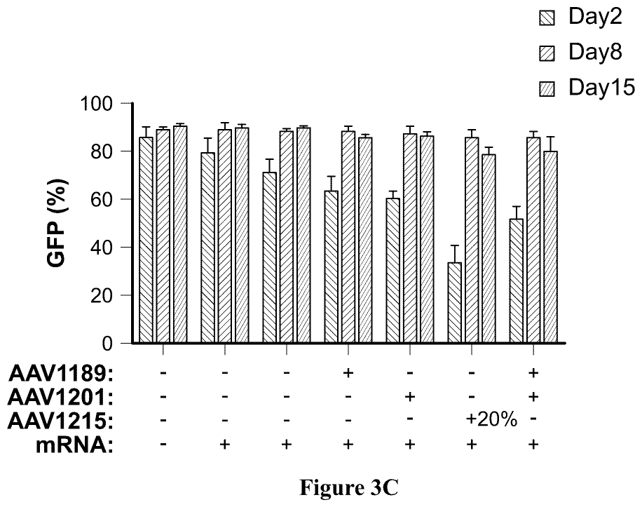

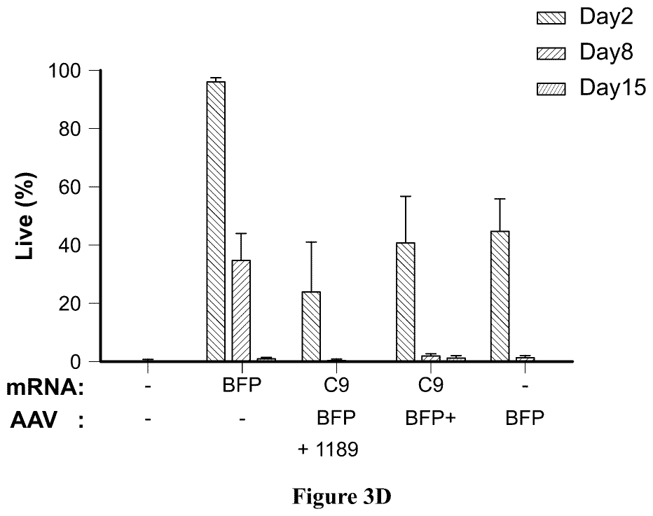

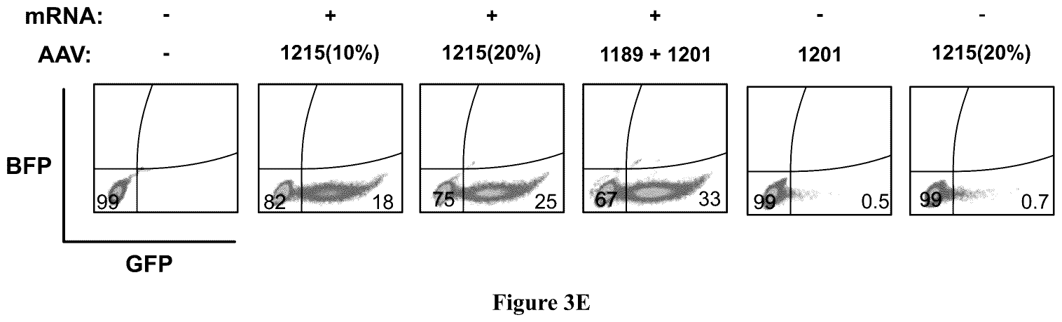

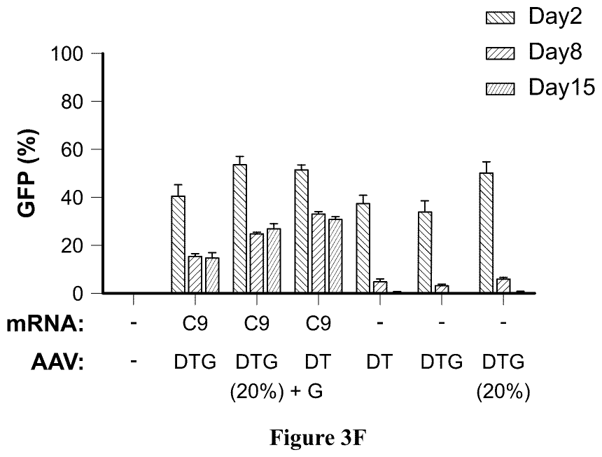

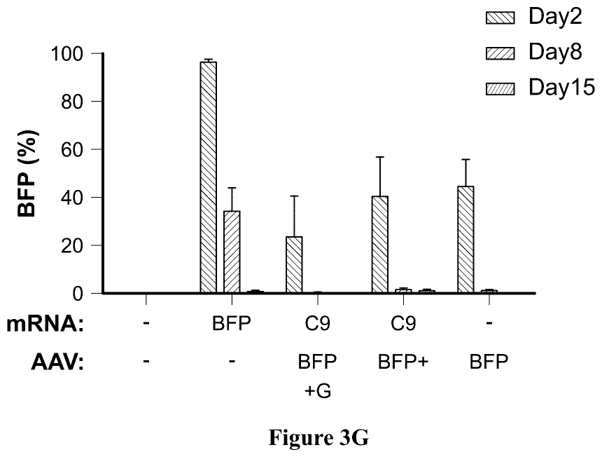

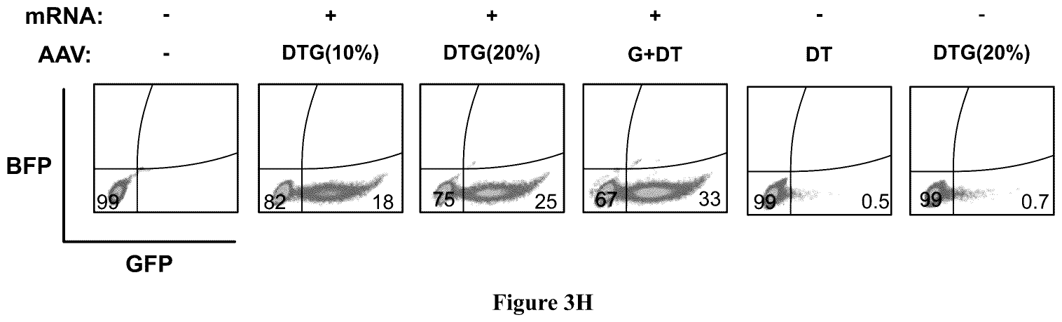

FIG. 3A depicts the scAAV guide RNA vector (#1189), donor template (#1201), and AAV vector containing both guide and donor sequences (#1215). Both #1201 and #1215 AAVs have the PAM site mutated to abolish cleavage by guide.

Primary human CD3 T cells were cultured as described in Example 1 and transfected with 1 μg of Cas9 mRNA followed by transduction with either vector #1189 and #1201 or #1215 AAV vectors. FIG. 3B shows the % GFP at days 2, 8, and 15, and FIG. 3C shows the cell viability at days 2, 8, and 15 under the specified conditions. 10% and 20% represent the % of culture volume #1215 AAV was added at. All other AAVs were added at 10% of culture volume. FIG. 3D depicts BFP expression when Cas9, guide and MND.BFP vector with no homology arms were delivered. FIG. 3E shows representative FACS plots showing GFP expression at Day 15. N=3 and represents the number of independent experiments performed using cells from 3 donors. FIG. 3F shows the % GFP at days 2, 8, and 15, and FIG. 3G depicts BFP expression when Cas9, guide and MND.BFP vector with no homology arms were delivered. FIG. 3H shows representative FACS plots showing GFP expression at day 15. N=3 and represents the number of independent experiments performed using cells from 3 donors.

Examples 2 and 3 show that using the T7 endonuclease assay, frequencies of 85% and 73% were achieved with TALENs and CRISPR/Cas systems, respectively. These examples also show methods that result in 70% homology directed repair (HDR) in T cells when the nucleases were co-delivered with an AAV donor template.

### Alternative 4

### Editing of Mobilized Adult CD34⁺ Cells with TALEN and AAV Co-Delivery

This alternative demonstrates methods for editing mobilized adult CD34⁺ cells using co-delivery of TALEN and AAV as described in some alternatives herein.

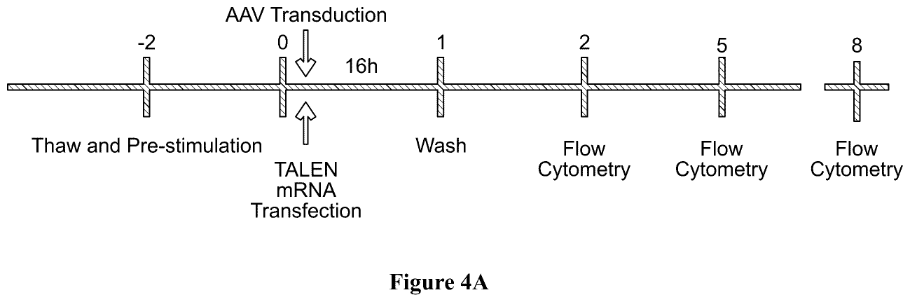

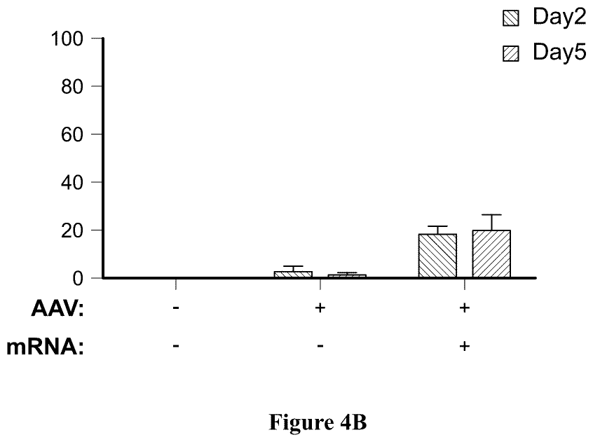

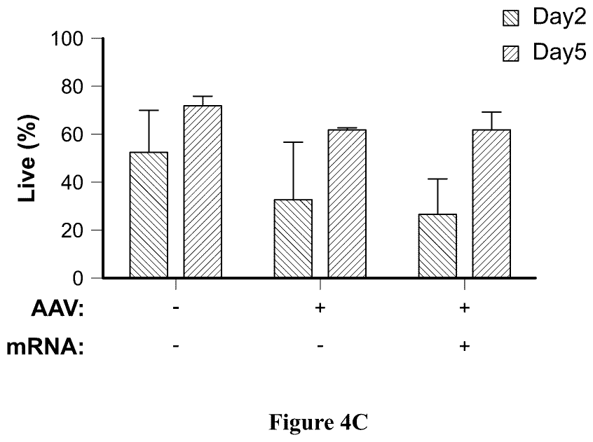

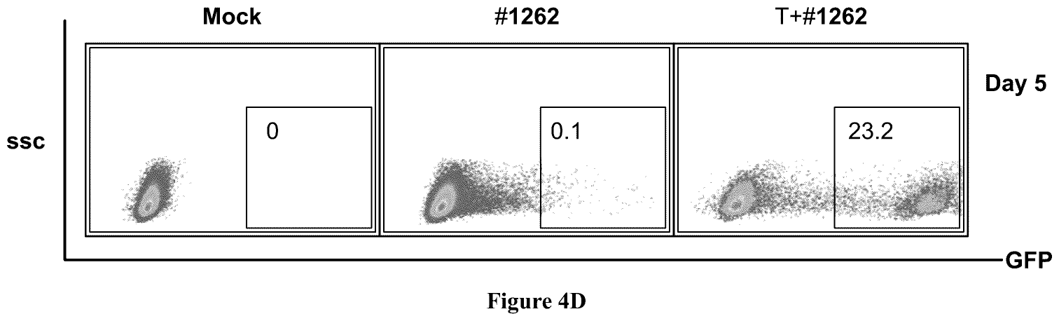

CD34⁺ cells were transfected as described in Example 2 with TALEN and AAV. FIG. 4A shows the timeline for the experimental conditions, where the cells were analyzed at days 2, 5, and 8 with flow cytometry. FIG. 4B shows the % GFP at days 2 and 5, and the cell viability is depicted in FIG. 4C. FIG. 4D shows representative FACS plots showing GFP expression at day 5.

This alternative demonstrates that the co-delivery of TALEN and AAV induces HDR in adult human mobilized CD34⁺ cells.

### Alternative 5

### Off-Target Cleavage with TALEN

This alternative provides potential off-target cleavage sites for TALENs.

T cells were transfected with 1 μg of forward and reverse WAS TALEN. Five days post-transfection, genomic DNA was extracted, endogenous WAS locus and predicted off target loci were amplified and colony sequenced. Off-targets were predicted using Prognos software. As shown in Table 1 below, potential off-target cleavage sites for TALENs identified using the Prognos software were amplified and sequenced, with no evidence of off-target cleavage observed at any of the predicted loci.

| | TALEN | | | | Genomic | Closest | |
|---|---|---|---|---|---|---|---|
| Ranking | Score | Orientation | Mismatches | Chr Name | Region | Gene | % Cleavage |
| 1 | 100 | L-17-R | 0_0 | ChrX | Intron | WAS | 94% (32/34) |
| 2 | 60.25 | R-26-L | 6_2 | Chr6 | Intergenic | LRFN2 | 0% (0/30) |
| 3 | 59.44 | R-25-L | 6_3 | Chr18 | Intron | DLGAP1 | 0% (0/30) |
| 4 | 59.18 | R-30-L | 5_3 | Chr12 | Exon | MAGOHB | 0% (0/30) |
| 5 | 56.42 | R-30-R | 5_3 | Chr7 | Intergenic | INHBA | 0% (0/30) |
| 6 | 56.15 | L-13-R | 6_3 | Chr1 | Intergenic | SIKE1 | 0% (0/30) |
| 7 | 55.97 | R-16-L | 6_3 | Chr8 | Intergenic | ANGPT1 | 0% (0/30) |
| 8 | 55.74 | R-15-R | 5_3 | Chr2 | Intron | RALB | 0% (0/30) |
| 12 | 55.18 | R-11-L | 5_4 | Chr3 | Intergenic | MIR548A3 | 0% (0/30) |

### Alternative 6

### Disruption of WAS Locus in Human CD34⁺ Cells Using TALENS or RNP

This alternative demonstrates the disruption of human CD34⁺ cells using TALEN or a ribonucleoprotein complex (RNP).

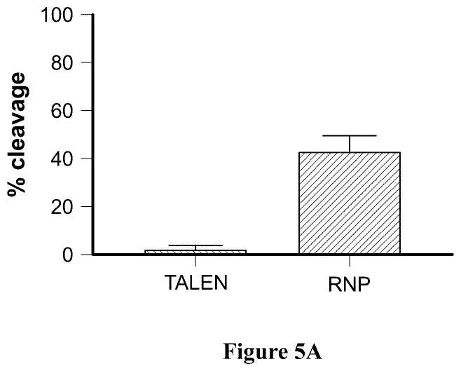

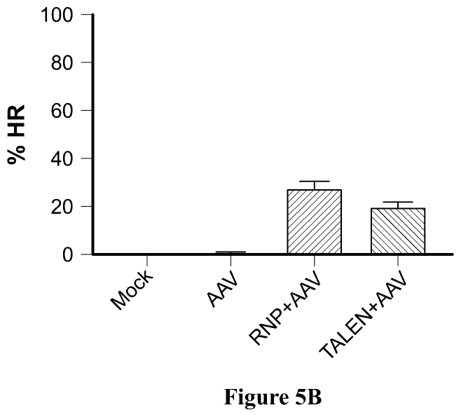

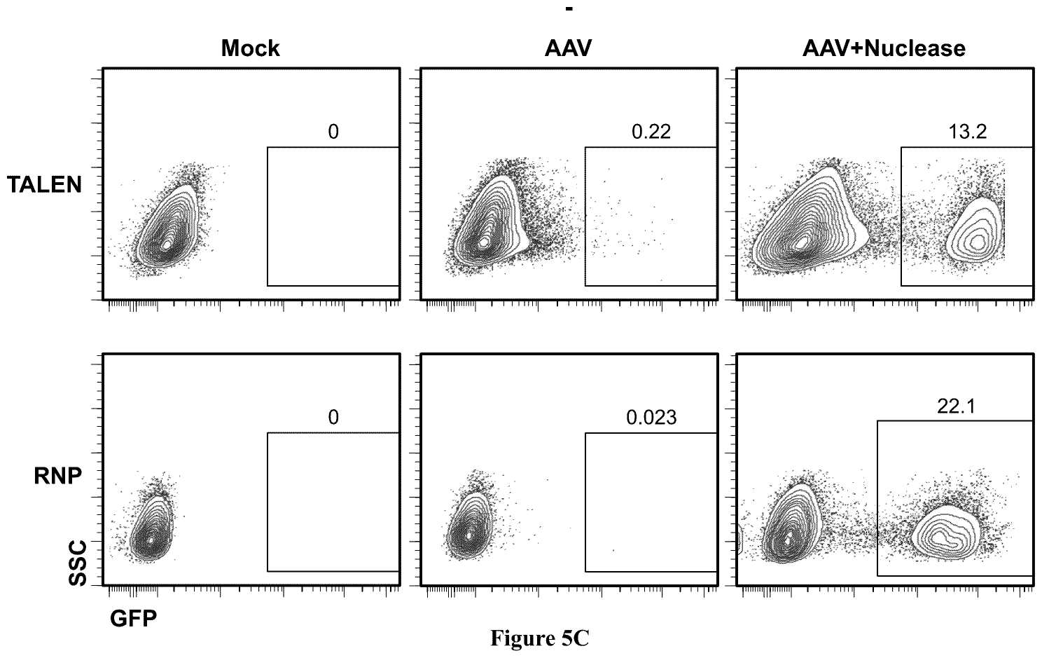

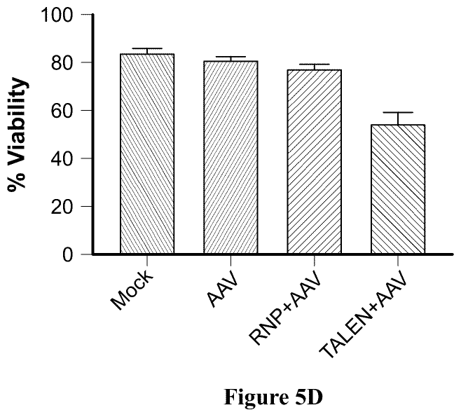

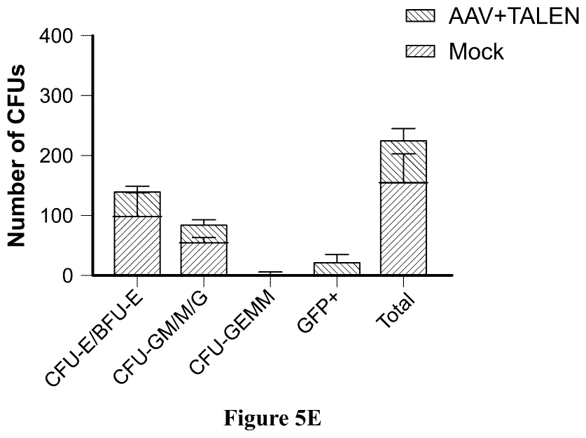

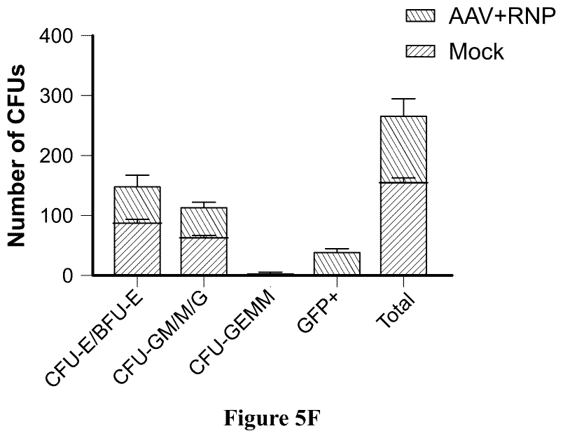

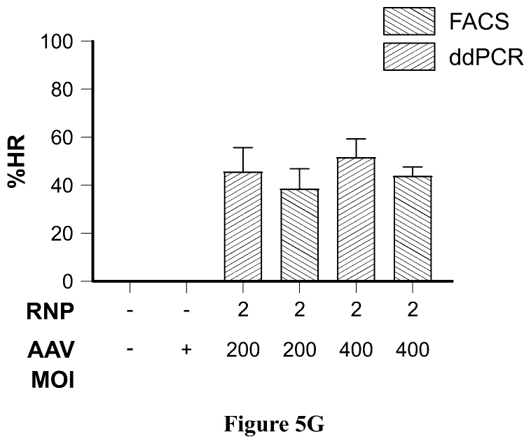

Mobilized human CD34⁺ cells were cultured in stem cell growth medium (SCGM) supplemented with thrombopoietin (TPO), stem cell factor (SCF), Fms-related tyrosine kinase 3 ligand (FLT-3L) (100 ng/mL) and IL-3 (60 ng/mL) for 48 hours, followed by electroporation using Neon electroporation system with either 1 μg of each TALEN monomer or ribonucleoprotein complex (RNP) of Cas9 protein and single guide RNA (sgRNA) mixed in 1:1.2 ratio. The sgRNA was purchased from Trilink BioTechnologies and has chemically modified nucleotides at the three terminal positions at 5′ and 3′ ends. The cells were cultured for 5 days and genomic DNA was extracted. The region surrounding the cut site for WAS TALEN and guide was amplified and cloned into pJET cloning vector. Colony sequencing was performed to quantify % cleavage at the cut site by analyzing the indels. FIG. 5A depicts the % cleavage of the WAS locus in the CD34⁺ cells for both TALEN and RNP. N=3 and represents the number of independent experiments performed using cells from three donors.

As shown in FIG. 5A, adult human mobilized CD34⁺ cells were cultured in SCGM media supplemented with TPO, SCF, FLT3L (100 ng/ml) and IL-3 (60 ng/ml) for 48 hours, followed by electroporation using Neon electroporation system with either 1 μg of each TALEN monomer or Ribonucleoprotein complex (RNP) of Cas9 protein and single guide RNA mixed in 1:1.2 ratio. The sgRNA was purchased from Trilink Biotechnologies and has chemically modified nucleotides at the three terminal positions at 5′ and 3′ ends. The cells were cultured for 5 days and genomic DNA was extracted. The region surrounding the cut site for WAS TALEN and guide was amplified and cloned into pJET cloning vector. Colony sequencing was performed to quantify % cleavage at the cut site by analyzing the indels. N=3 and represents the number of independent experiments performed using cells from 3 donors.

As shown in FIG. 5B, adult mobilized human CD34⁺ cells were cultured in SCGM media as described in FIG. 5A, followed by electroporation using Neon electroporation system with either TALEN mRNA or RNP complex (2 μg). AAV vector (MOI ranging from 62-300) carrying the donor template was added immediately after electroporation. Controls included un-manipulated cells (mock) and cells transduced with AAV only without transfection of a nuclease (AAV). Bar graphs depict % GFP at day 5, indicative of HDR. n=8 for RNP, n=15 for TALEN, and represents >4 donors.

Shown in FIG. 5C, are FACS plots depicting GFP expression from Mock, AAV or AAV plus TALEN treated CD34⁺ cells (top row) or AAV+RNP treated cells (bottom row) 5 days post editing.

Shown in FIG. 5D, bar graphs represent viability of mock and edited cells 2 days post editing. N=8 for RNP, n=12 for TALEN and represents >4 independent donors.

Shown in FIG. 5E, are results from a CFU assay for TALEN edited CD34⁺ cells. TALEN edited and mock cells were plated one day post editing onto Methocult media for colony formation unit (CFU) assay. Briefly, 500 cells were plated in duplicate in Methocult H4034 media (Stemcell Technologies), incubated at 37° C. for 12-14 days and colonies enumerated based on their morphology and GFP expression. CFU-E: Colony forming unit erythroid, M: Macrophage, GM: Granulocyte, macrophage, G: Granulocyte, GEMM: Granulocyte, erythroid, macrophage, megakaryocyte, BFU-E: Burst forming unit erythroid. n=3 independent donors. Data are presented as mean±SEM.

Shown in FIG. 5F, are results from a CFU assay for RNP edited CD34+ cells. RNP edited and mock cells were plated one day post editing onto Methocult media for colony formation unit (CFU) assay. Briefly, 500 cells were plated in duplicate in Methocult H4034 media (Stemcell Technologies), incubated at 37° C. for 12-14 days and colonies enumerated based on their morphology and GFP expression. CFU-E: Colony forming unit erythroid, M: Macrophage, GM: Granulocyte, macrophage, G: Granulocyte, GEMM: Granulocyte, erythroid, macrophage, megakaryocyte, BFU-E: Burst forming unit erythroid. n=3 experiments and 2 donors. Data are presented as mean±SEM.

Shown in FIG. 5G are results from the digital droplet PCR assay for determining HDR. Genomic DNA was isolated from hematopoietic stem and progenitor cells (HSPCs) using a DNeasy Blood and Tissue kit (Qiagen). To assess editing rates, “in-out” droplet digital PCR was performed with the forward primer binding within the AAV insert and the reverse primer binding the WAS locus outside the region of homology. A control amplicon of similar size was generated for the ActB gene to serve as a control. All reactions were performed in duplicate. The PCR reactions were partitioned into droplets using a QX200 Droplet Generator (Bio-Rad). Amplification was performed using ddPCR Supermix for Probes without UTP (Bio-Rad), 900 nM of primers, 250 nM of Probe, 50 ng of genomic DNA, and 1% DMSO. Droplets were analyzed on the QX200 Droplet Digital PCR System (Bio-Rad) using QuantaSoft software (Bio-Rad). Data are presented as mean±SEM.

### Alternative 7

### Editing of the WAS Locus in CD34⁺ HSCs with TALEN mRNA/RNP and AAV Co-Delivery

This alternative demonstrates editing of the WAS locus in CD34⁺ hematopoietic stem cells using co-delivery of TALEN mRNA or RNP and an AAV donor template.

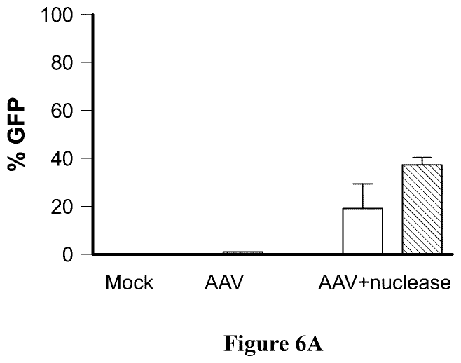

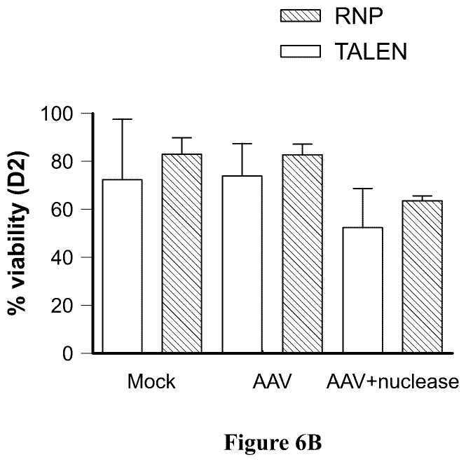

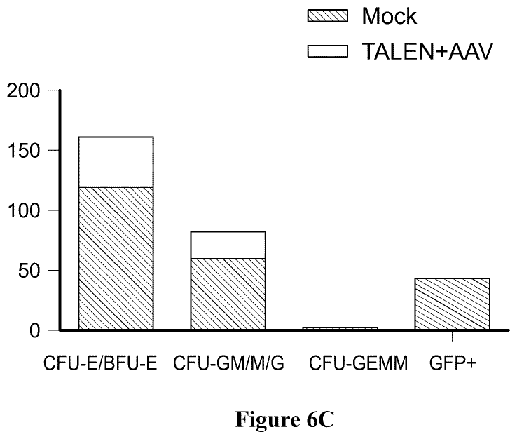

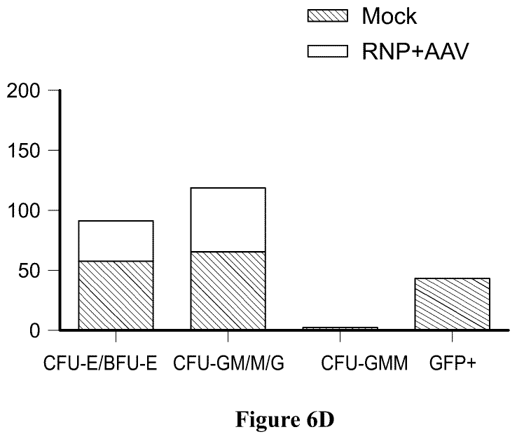

Adult mobilized human CD34⁺ cells were cultured in SCGM media as described in Example 6, followed by electroporation using Neon electroporation system with either TALEN mRNA or RNP complex. AAV vector carrying the donor template was added immediately after electroporation. Controls included un-manipulated cells (mock) and cells transduced with AAV only without transfection of a nuclease (AAV). FIG. 6A shows % GFP at day 5 indicative of HDR. FIG. 6B shows the viability of edited cells two days post editing. N=7 for TALEN and N=3 for RNP and represent independent donors. Edited and mock cells were plated one day post editing onto Methocult media for colony formation unit (CFU) assay. Briefly, 500 cells were plated in duplicate in Methocult H4034 media (Stemcell Technologies, incubated at 37° C. for 12-14 days and colonies enumerated based on their morphology and GFP expression. FIG. 6C shows data from TALEN and FIG. 6D shows data from RNP. CFU-E: Colony forming unit erythroid, M: Macrophage, GM: Granulocyte, macrophage, G: Granulocyte, GEMM: Granulocyte, erythroid, macrophage, megakaryocyte, BFU-E: Burst forming unit erythroid.

### Alternative 8

### Engraftment of Edited Human CD34⁺ Cells into Immune Deficient NSG Mice

This alternative demonstrates the use of the edited cells in immune deficient mice.

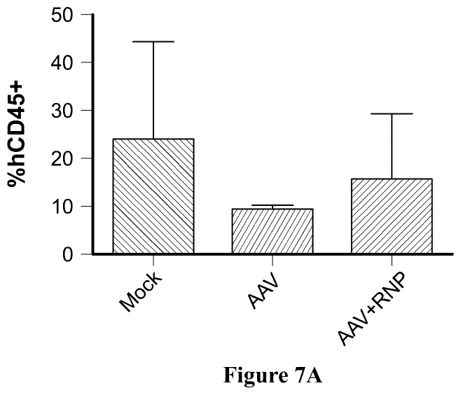

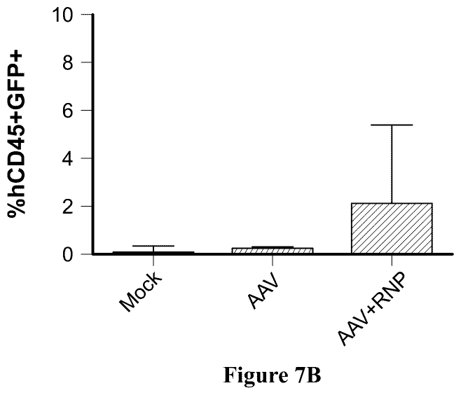

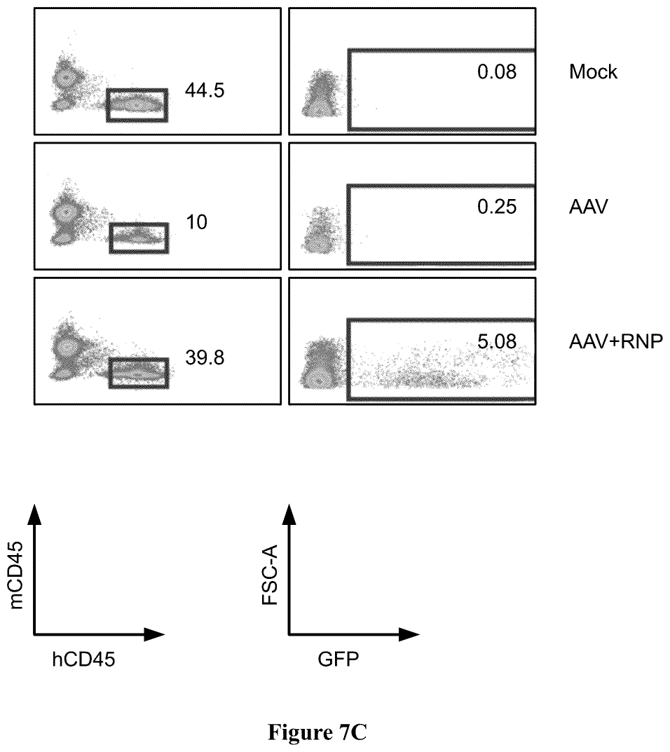

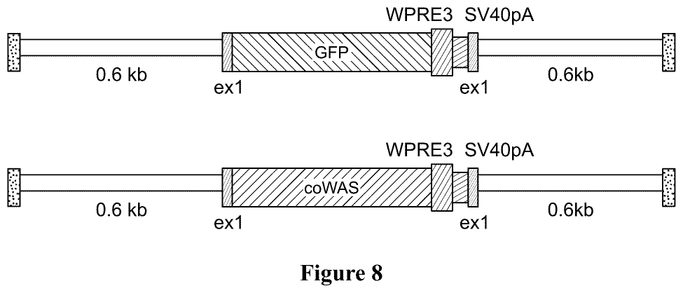

Two million human CD34⁺ cells that were either untreated, AAV treated, or treated with AAV and RNP were injected into immune deficient non-obese diabetic (NOD) severe combined immunodeficiency (scid) gamma (NSG) mice preconditioned with 25 mg/kg busulfan. The mice were sacrificed 10 weeks post-transplant and bone marrow was harvested. FIG. 7A depicts total engraftment of edited cells as defined by expression of human CD45 marker. FIG. 7B Illustrates % GFP+ cells within the engrafted cells. FIG. 7C shows FACS plots from representative mice.

### Alternative 9

### Percent HR in Cells for Engraftment

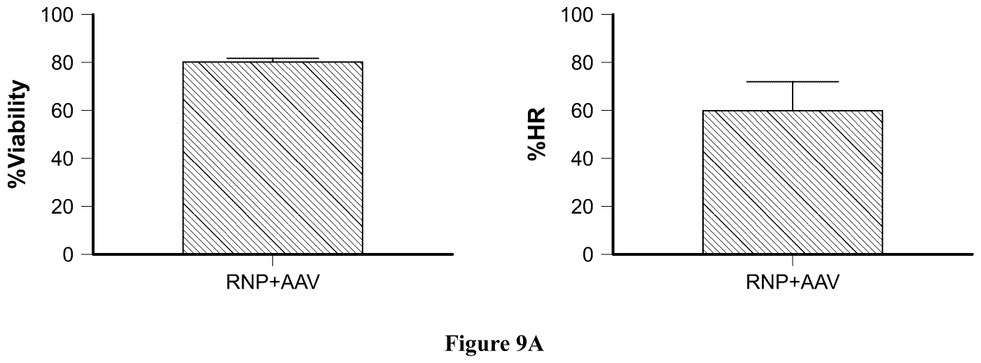

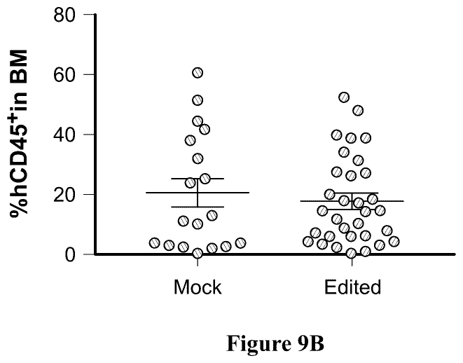

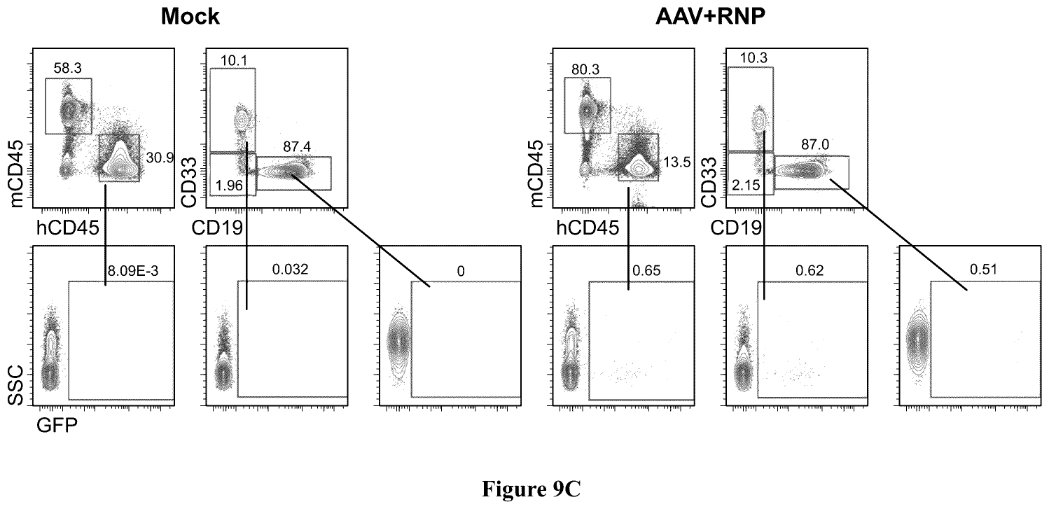

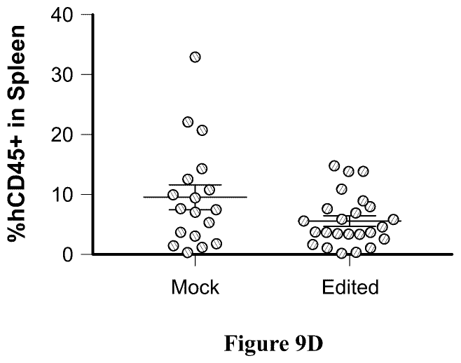

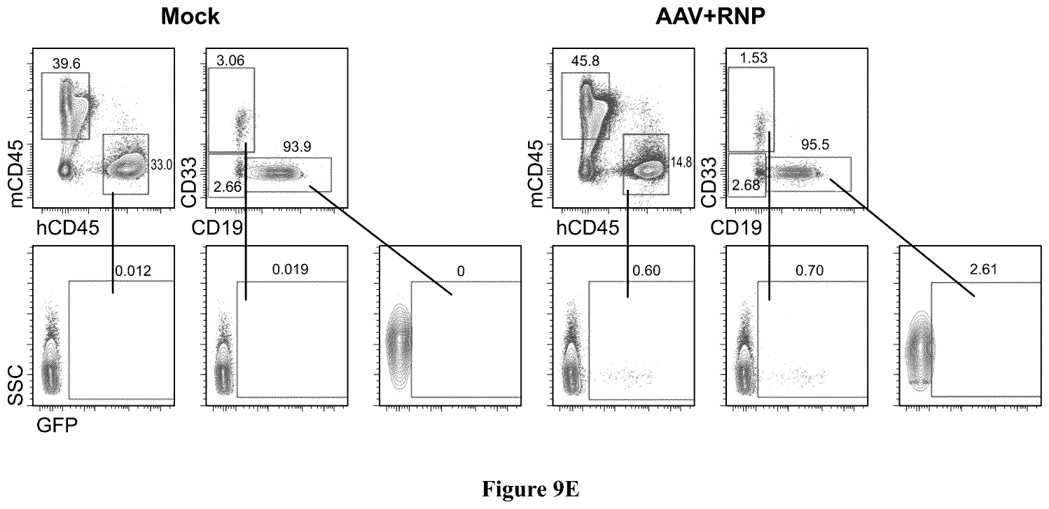

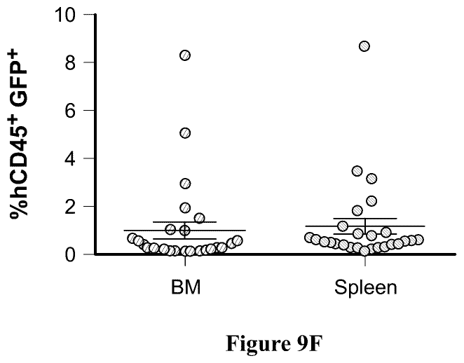

Shown in FIG. 9A is the cell viability and % HR in input cells used for engraftment in NSG mice. Adult mobilized human CD34+ cells were cultured in SCGM media as previously described with the exception that IL-6 (100 ng/ml) was added instead of IL-3. The cells were electroporated with 1 μg of RNP complex and AAV at an MOI of 62. Data are presented as mean±SEM. N=5 and represents independent experiments. FIG. 9B shows the results from engraftment of edited cells in bone marrow of transplanted NSG mice. Six to 10 week old NSG mice were treated with 25 or 35 mg/kg of BUSULFEX (Henry Schein Inc.) via intraperitoneal injection, diluted 1:1 in phosphate-buffered saline. Twenty-four hours later, 2×10⁶ mock or gene edited hematopoietic stem cells (cultured as described in FIG. 9A) in phosphate-buffered saline were delivered via retro-orbital injection. Animals were euthanized 10 to 16 weeks post-transplant, bone marrow and spleens were harvested and analyzed for human cell engraftment. Dot plot depicts total engraftment of edited cells as defined by expression of human CD45 marker in BM of sacrificed mice. Dots represent individual mice. Data are presented as mean±SEM. FIG. 9C shows the result of engraftment of edited Cells in bone marrow of NSG mice 16 weeks post transplantation. Representative flow plots of cells harvested from the bone marrow of NSG mice 16 weeks following cell transplantation. On left panel shows the data of bone marrow harvested from mouse transplanted with untreated cells. On right, bone marrow harvested from mouse transplanted with cells treated with AAV plus RNP. On the top row of the figure, from left to right: hCD45:mCD45 chimerism, human CD45-gated CD33+ and CD19+ staining. Bottom row, from left to right: GFP expression among hCD45+, CD33+ and CD19+ cells. FIG. 9D Engraftment of edited cells in spleens of transplanted mice. Graph depicts total engraftment of edited cells at 10-16 weeks as defined by expression of human CD45 marker in spleens of sacrificed mice. Dots represent individual mice. FIG. 9E shows results from engraftment of edited cells in spleens of NSG Mice. Representative flow plots of cells harvested from the spleens of NSG mice 16 weeks following cell transplantation are shown. On left, bone marrow harvested from mouse transplanted with untreated cells. On right, bone marrow harvested from mouse transplanted with cells treated with AAV plus RNP. Top row, from left to right: hCD45:mCD45 chimerism, Human CD45-gated CD33+ and CD19+ staining. Bottom row, from left to right: GFP expression among hCD45+, CD33+ and CD19+ cells. FIG. 9F Engraftment of GFP+ cells in NSG mice. HDR-editing rate (% GFP+) among hCD45+ cells recovered from the bone marrow and spleens of NSG mice sacrificed at 10-16 weeks. Mean±SEM shown on graph.

### Alternative 10

### Engraftment of Edited Cells in NSGW41 Mice

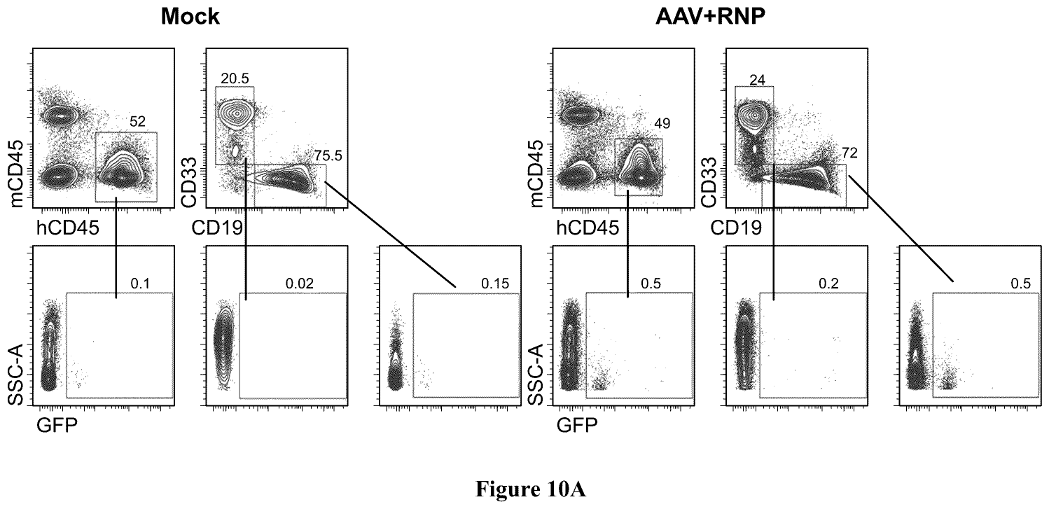

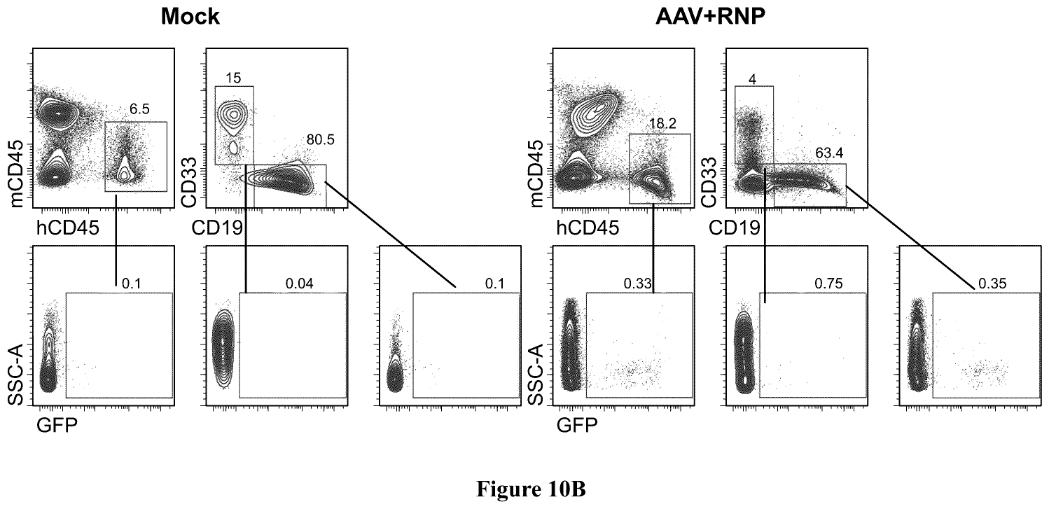

Shown in FIG. 10A are the results from engraftment of edited cells in bone marrows of NSGW41 mice. Representative flow plots of cells harvested from the bone marrows of NSGW41 mice 16 weeks following cell transplantation. On left, bone marrow harvested from mouse transplanted with untreated cells. On right, bone marrow harvested from mouse transplanted with cells treated with AAV plus RNP. Top row, from left to right: hCD45:mCD45 chimerism, Human CD45-gated CD33⁺ and CD19⁺ staining. Bottom row, from left to right: GFP expression among hCD45⁺, CD33⁺ and CD19⁺ cells.

Shown in FIG. 10B are the results from engraftment of edited cells in spleens of NSGW41 mice. Representative flow plots of cells harvested from the spleens of NSGW41 mice 16 weeks following cell transplantation. On left, bone marrow harvested from mouse transplanted with untreated cells. On right, bone marrow harvested from mouse transplanted with cells treated with AAV plus RNP. Top row, from left to right: hCD45:mCD45 chimerism, Human CD45-gated CD33⁺ and CD19⁺ staining. Bottom row, from left to right: GFP expression among hCD45⁺, CD33⁺ and CD19⁺ cells.

### Alternative 11

### Engraftment of Edited Cells in NSGW41 Mice

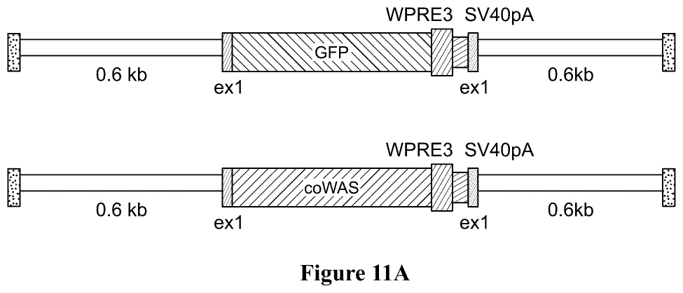

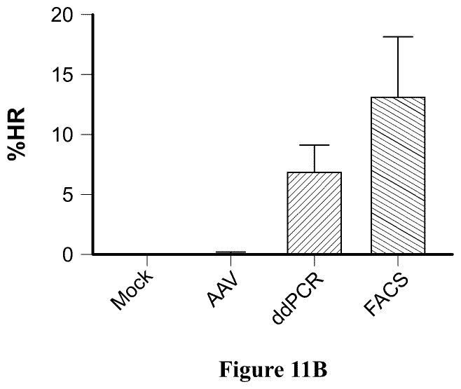

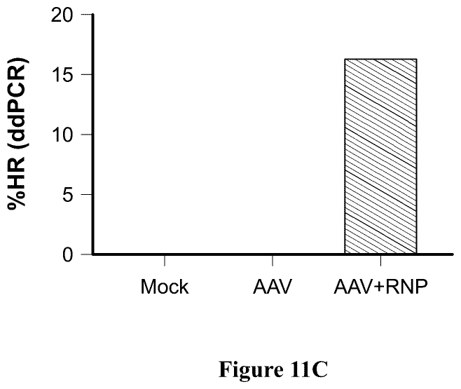

FIG. 11A shows the design of AAV vectors expressing cDNA for GFP or codon optimized WAS gene from the endogenous promoter. AAV vectors with 0.6 kb homology arms flanking either a promoterless GFP (top) or WAS cDNA (bottom) followed by a shorter WPRE, designated WPRE3 followed by SV40 polyadenylation signal. As shown in FIG. 11B is the results from the digital droplet PCR to determine editing rates in WAS targeted promoterless GFP construct. In-out droplet digital PCR was performed as previously described. Droplets were analyzed on the QX200 Droplet Digital PCR System (Bio-Rad) using QuantaSoft software (Bio-Rad). All experiments were performed on female donors. The ddPCR rates were about half of HR rates by FACS suggesting that only one X chromosome was being targeted, n=3 and represents three independent experiments and donors. Data are presented as mean±SEM. As shown in FIG. 11C is the results from the digital droplet PCR to determine editing rates with WAS targeted promoterless coWAS expressing AAV vectors.

### MORE ALTERNATIVES

Further aspects of the disclosure are realized in the following numbered alternatives.

In some alternatives, a nucleic acid for homology directed repair (HDR) of Wiskott-Aldrich Syndrome (WAS) gene is provided, the nucleic acid comprising: a first sequence encoding a WAS gene; a second sequence encoding one or more guide RNA-cleavage sites; and a third sequence encoding one or more nuclease-binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the nucleic acid further comprises one or more enhancer elements. In some alternatives, the nucleic acid further comprises homology arm sequences. In some alternatives, the nucleic acid further comprises nucleic acid sequence encoding a promoter.

In some alternatives, a vector for promoting HDR of WAS protein (WASp) expression in a cell is provided, the vector comprising: a first sequence encoding a WAS gene; a second sequence encoding one or more guide RNA-cleavage sites; and a third sequence encoding one or more nuclease-binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the vector further comprises one or more enhancer elements. In some alternatives, the vector is an adeno-associated viral vector (AAV). In some alternatives, the vector is a self-complementary AAV (scAAV). In some alternatives, the cell is a human cell. In some alternatives, the cell is a primary cell. In some alternatives, the cell is an autologous cell. In some alternatives, the cell is a T cell. In some alternatives, the cell is a hematopoietic stem cell (HSC). In some alternatives, the cell is a CD34⁺ HSC.

In some alternatives, a system for promoting HDR of WAS protein (WASp) expression in a cell is provided, where're the system comprises a vector of any one of the alternatives herein and a nucleic acid encoding a nuclease. In some alternatives, the vector comprises a first sequence encoding a WAS gene; a second sequence encoding one or more guide RNA-cleavage sites; and a third sequence encoding one or more nuclease-binding sites. In some alternatives, the WAS gene comprises the nucleic acid sequence set forth in SEQ ID NO: 4. In some alternatives, the second sequence comprises the nucleic acid sequence set forth in SEQ ID NO: 17. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the vector further comprises one or more enhancer elements. In some alternatives, the vector is an adeno-associated viral vector (AAV). In some alternatives, the vector is a self-complementary AAV (scAAV). In some alternatives, the cell is a human cell. In some alternatives, the cell is a primary cell. In some alternatives, the cell is an autologous cell. In some alternatives, the cell is a T cell. In some alternatives, the cell is a hematopoietic stem cell (HSC). In some alternatives, the cell is a CD34⁺ HSC. In some alternatives of the system, the nuclease is a TALEN nuclease. In some alternatives of the system, the nuclease is a Cas nuclease. In some alternatives of the system, the vector and nucleic acid are configured for co-delivery to the cell. In some alternatives of the system, co-delivery to the cell modifies endogenous WAS locus. In some alternatives of the system, the cell is a primary human hematopoietic cell.

In some alternatives, a cell for expressing a WASp is provided, the cell comprising: a nucleic acid, which comprises: a first sequence encoding a WAS gene; a second sequence encoding a promoter; a third sequence encoding one or more guide RNA cleavage sites; and a fourth sequence encoding one or more nuclease binding sites. In some alternatives, the nucleic acid is in a vector. In some alternatives, the vector are an AAV. In some alternatives, the AAV are a scAAV. In some alternatives, the cell are a human cell. In some alternatives, the cell are a primary cell. In some alternatives, the cell are an autologous cell. In some alternatives, the cell are a T cell. In some alternatives, the cell are a HSC. In some alternatives, the cell are a CD34⁺ HSC.

In some alternatives, a method of promoting HDR of a WAS gene in a subject in need thereof is provided, the method comprising: administering to a subject the cell of any one of alternatives provided herein or a vector of any one of the alternatives provided herein, and administering to the subject a nuclease. In some alternatives, the nuclease is a TALEN nuclease. In some alternatives, the nuclease is a Cas nuclease. In some alternatives, the nuclease is co-administered to the subject with the cell or with the vector. In some alternatives, the cell is from the subject and, wherein the cell is genetically modified by introducing the nucleic acid of any one of alternatives of any one of the alternatives herein or the vector of any one of the alternatives herein into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34⁺ HSC. In some alternatives, the subject is male. In some alternatives, the subject is suffering from Wiskott-Aldrich syndrome (WAS). In some alternatives, the subject is suffering from X-linked thrombocytopenia (XLT).

In some alternatives, a method of treating, inhibiting, or ameliorating WAS and/or XLT or disease symptoms associated with WAS and/or XLT in a subject in need thereof is provided, the method comprising: administering to a subject the cell of any one of the alternatives herein or a vector of any one of the alternatives herein; administering to the subject a nuclease; and optionally identifying the subject as one that would benefit from receiving a therapy for WAS and/or XLT or disease symptoms associated with WAS and/or XLT and/or, optionally measuring an improvement in the progression of WAS and/or XLT or an improvement in a disease symptom associated with WAS and/or XLT in said subject. In some alternatives, the nuclease is a TALEN nuclease. In some alternatives, the nuclease is a CRISPR/Cas nuclease. In some alternatives, the nuclease is co-administered to the subject with the cell or with the vector. In some alternatives, the cell is from the subject, wherein the cell is genetically modified by introducing the nucleic acid of any one of alternatives 1-8 or the vector of any one of the alternatives herein into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34⁺ HSC. In some alternatives, the subject is male. In some alternatives, the method improves thrombocytopenia. In some alternatives, the method increases platelet counts.

In some alternatives, a nucleic acid for homology directed repair (HDR) of Wiskott-Aldrich Syndrome (WAS) gene is provided, the nucleic acid comprising: a first sequence encoding a WAS gene, a second sequence encoding one or more guide RNA cleavage sites; and a third sequence encoding one or more nuclease binding sites. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the nucleic acid further comprises one or more enhancer elements. n some alternatives, the nucleic acid further comprises homology arm sequences. n some alternatives, the nucleic acid further comprises a nucleic acid sequence encoding a promoter.

In some alternatives, a vector for promoting HDR of WAS protein (WASp) expression in a cell is provided, the vector comprising a first sequence encoding a WAS gene, a second sequence encoding one or more guide RNA cleavage sites; and a third sequence encoding one or more nuclease binding sites. In some alternatives, the one or more nuclease binding sites comprises a forward and reverse transcription activator-like effector nuclease (TALEN) binding site. In some alternatives, the one or more nucleic binding sites is a clustered regularly interspaced short palindromic repeats (CRISPR) associated protein 9 (Cas9) binding site. In some alternatives, the vector further comprises one or more enhancer elements. In some alternatives, the vector is an adeno-associated viral vector (AAV). In some alternatives, the vector is a self-complementary AAV (scAAV). In some alternatives, the cell is a human cell. In some alternatives, the cell is a primary cell. In some alternatives, the cell is an autologous cell. In some alternatives, the cell is a T cell. In some alternatives, the cell is a hematopoietic stem cell (HSC). In some alternatives, the cell is a CD34+ HSC.

In some alternatives, a system for promoting HDR of WAS protein (WASp) expression in a cell is provided, the system comprising a vector of any one of the alternatives herein and a nucleic acid encoding a nuclease. In some alternatives, the nuclease is a TALEN nuclease. In some alternatives, the nuclease is a Cas nuclease. In some alternatives, the vector and nucleic acid are configured for co-delivery to the cell. In some alternatives, co-delivery to the cell modifies endogenous WAS locus. In some alternatives, the cell is a primary human hematopoietic cell.

In some alternatives, a cell for expressing a WASp is provided, the cell comprising: a nucleic acid, which comprises a first sequence encoding a WAS gene, a second sequence encoding a promoter, a third sequence encoding one or more guide RNA cleavage sites; and a fourth sequence encoding one or more nuclease binding sites. In some alternatives, the nucleic acid is in a vector. In some alternatives, the vector is an AAV. In some alternatives, the AAV is a scAAV. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34+ HSC.

In some alternatives a method of promoting HDR of a WAS gene in a subject in need thereof, the method comprising: administering to a subject the cell or a vector of any one of the alternatives herein; and administering to the subject a nuclease. In some alternatives, the nuclease is a TALEN nuclease. In some alternatives, the nuclease is a Cas nuclease. In some alternatives, the nuclease is co-administered to the subject with the cell or with the vector. In some alternatives, the cell is from the subject and, wherein the cell is genetically modified by introducing the nucleic acid or the vector of any one of the alternatives herein into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34+ HSC. In some alternatives, the subject is male. In some alternatives, the subject is suffering from Wiskott-Aldrich syndrome (WAS). In some alternatives, the subject is suffering from X-linked thrombocytopenia (XLT).

In some alternatives, a method of treating, inhibiting, or ameliorating WAS and/or XLT or disease symptoms associated with WAS and/or XLT in a subject in need thereof is provided, the method comprising: administering to a subject the cell or a vector of any one of the alternatives herein, administering to the subject a nuclease; and optionally identifying the subject as one that would benefit from receiving a therapy for WAS and/or XLT or disease symptoms associated with WAS and/or XLT and/or, optionally measuring an improvement in the progression of WAS and/or XLT or an improvement in a disease symptom associated with WAS and/or XLT in said subject. In some alternatives, the nuclease is a TALEN nuclease. In some alternatives, the nuclease is a CRISPR/Cas nuclease. In some alternatives, the nuclease is co-administered to the subject with the cell or with the vector. In some alternatives, the cell is from the subject, wherein the cell is genetically modified by introducing the nucleic acid or the vector of any one of the alternatives into the cell. In some alternatives, the administering is performed by adoptive cell transfer. In some alternatives, the cell is a human cell. In some alternatives, the cell is a primary cell. In some alternatives, the cell is an autologous cell. In some alternatives, the cell is a T cell. In some alternatives, the cell is a HSC. In some alternatives, the cell is a CD34+ HSC. In some alternatives, the subject is male. In some alternatives, the method improves thrombocytopenia. In some alternatives, the method increases platelet counts.

It is to be understood that the description, specific examples and data, while indicating exemplary alternatives, are given by way of illustration and are not intended to limit the various alternatives of the present disclosure. Various changes and modifications within the present disclosure will become apparent to the skilled artisan from the description and data contained herein, and thus are considered part of the various alternatives of this disclosure.

APPENDIX I
Sequence Identification Numbers, Identifying Descriptions, and Sequences

SEQ ID NO: 1 WAS TALEN forward
ATGGCGCCGCGGCCTCCTAAGAAGAAGCGGAAAGTCGAATTCGTGGATCTGCGA
ACACTGGGCTATAGCCAGCAGCAGCAGGAGAAGATCAAACCCAAGGTGAGGTC
CACAGTCGCACAGCACCATGAAGCCCTGGTGGGCCACGGGTTCACTCACGCTCA
TATTGTCGCACTGTCTCAGCATCCAGCCGCTCTGGGAACCGTGGCAGTCACATAC
CAGCACATCATTACTGCCCTGCCCGAGGCTACCCATGAAGACATCGTGGGAGTC
GGCAAACAGTGGAGCGGCGCACGGGCCCTGGAGGCTCTGCTGACCGACGCAGG
GGAACTGAGAGGACCCCCTCTGCAGCTGGATACAGGGCAGCTGGTGAAGATTGC
TAAGAGGGGAGGGGTGACAGCAATGGAAGCCGTCCACGCAAGCAGGAACGCAC
TGACAGGGGCCCCCCTGAACCTGACCCCGGACCAAGTGGTGGCTATCGCCAGCC
ACGATGGCGGCAAGCAAGCGCTCGAAACGGTGCAGCGGCTGTTGCCGGTGCTGT
GCCAGGACCATGGCCTGACCCCGGACCAAGTGGTGGCTATCGCCAGCAACGGTG
GCGGCAAGCAAGCGCTCGAAACGGTGCAGCGGCTGTTGCCGGTGCTGTGCCAGG
ACCATGGCCTGACCCCGGACCAAGTGGTGGCTATCGCCAGCCACGATGGCGGCA
AGCAAGCGCTCGAAACGGTGCAGCGGCTGTTGCCGGTGCTGTGCCAGGACCATG
GCCTGACCCCGGACCAAGTGGTGGCTATCGCCAGCCACGATGGCGGCAAGCAAG
CGCTCGAAACGGTGCAGCGGCTGTTGCCGGTGCTGTGCCAGGACCATGGCCTGA
CTCCGGACCAAGTGGTGGCTATCGCCAGCAACGGTGGCGGCAAGCAAGCGCTCG
AAACGGTGCAGCGGCTGTTGCCGGTGCTGTGCCAGGACCATGGCCTGACCCCGG
ACCAAGTGGTGGCTATCGCCAGCAACATTGGCGGCAAGCAAGCGCTCGAAACGG
TGCAGCGGCTGTTGCCGGTGCTGTGCCAGGACCATGGCCTGACTCCGGACCAAG
TGGTGGCTATCGCCAGCAACAATGGCGGCAAGCAAGCGCTCGAAACGGTGCAGC
GGCTGTTGCCGGTGCTGTGCCAGGACCATGGCCTGACCCCGGACCAAGTGGTGG
CTATCGCCAGCAACATTGGCGGCAAGCAAGCGCTCGAAACGGTGCAGCGGCTGT
TGCCGGTGCTGTGCCAGGACCATGGCCTGACCCCGGACCAAGTGGTGGCTATCG
CCAGCAACATTGGCGGCAAGCAAGCGCTCGAAACGGTGCAGCGGCTGTTGCCGG
TGCTGTGCCAGGACCATGGCCTGACTCCGGACCAAGTGGTGGCTATCGCCAGCA
ACGGTGGCGGCAAGCAAGCGCTCGAAACGGTGCAGCGGCTGTTGCCGGTGCTGT
GCCAGGACCATGGCCTGACCCCGGACCAAGTGGTGGCTATCGCCAGCAACGGTG
GCGGCAAGCAAGCGCTCGAAACGGTGCAGCGGCTGTTGCCGGTGCTGTGCCAGG
ACCATGGCCTGACCCCGGACCAAGTGGTGGCTATCGCCAGCAACGGTGGCGGCA
AGCAAGCGCTCGAAACGGTGCAGCGGCTGTTGCCGGTGCTGTGCCAGGACCATG
GCCTGACCCCGGACCAAGTGGTGGCTATCGCCAGCCACGATGGCGGCAAGCAAG
CGCTCGAAACGGTGCAGCGGCTGTTGCCGGTGCTGTGCCAGGACCATGGCCTGA
CCCCGGACCAAGTGGTGGCTATCGCCAGCCACGATGGCGGCAAGCAAGCGCTCG
AAACGGTGCAGCGGCTGTTGCCGGTGCTGTGCCAGGACCATGGCCTGACCCCGG
ACCAAGTGGTGGCTATCGCCAGCCACGATGGCGGCAAGCAAGCGCTCGAAACGG
TGCAGCGGCTGTTGCCGGTGCTGTGCCAGGACCATGGCCTGACTCCGGACCAAG
TGGTGGCTATCGCCAGCAACAATGGCGGCAAGCAAGCGCTCGAAACGGTGCAGC
GGCTGTTGCCGGTGCTGTGCCAGGACCATGGCCTGACCCCGGACCAAGTGGTGG
CTATCGCCAGCAACGGTGGCGGCAAGCAAGCGCTCGAAAGCATTGTGGCCCAGC
TGAGCCGGCCTGATCCGGCGTTGGCCGCGTTGACCAACGACCACCTGGTCGCTCT
GGCTTGCCTGGGAGGACGCCCTGCTATGGACGCTGTGAAGAAAGGACTGCCCCA
CGCACCCGAACTGATTAGACGGGTGAACCGGAGAATCGGCGAGAGAACATCCC
ATAGGGTGGCAATCTCTAGAACTCAGCTGGTCAAGAGTGAACTGGAGGAAAAGA
AATCAGAGCTGCGCCACAAGCTGAAATACGTGCCTCATGAGTATATCGAACTGA
TCGAGATTGCTCGCAATTCAACCCAGGACCGGATCCTGGAAATGAAAGTGATGG
AGTTCTTTATGAAAGTCTACGGATATCGGGGGAAACACCTGGGAGGGAGCAGAA
AGCCAGATGGGGCCATCTACACAGTGGGATCCCCCATCGACTATGGCGTGATTG
TCGATACTAAAGCCTACAGCGGAGGCTATAACCTGCCTATCGGCCAGGCTGACG
AGATGCAGAGATACGTGGAGGAAAACCAGACCCGCAATAAGCATATTAACCCC
AATGAATGGTGGAAAGTGTATCCTAGCTCCGTCACAGAGTTCAAGTTTCTGTTCG
TGAGCGGACACTTTAAGGGCAACTACAAAGCACAGCTGACTAGGCTGAATCATA
TCACCAACTGCAATGGAGCCGTGCTGTCTGTCGAGGAACTGCTGATCGGGGGAG
AGATGATTAAGGCTGGCACACTGACTCTGGAGGAAGTGAGGCGCAAGTTCAACA
ATGGGGAAATCAACTTCTAA
SEQ ID NO: 2 WAS TALEN reverse
ATGGCGCCGCGGCCTCCTAAGAAGAAGCGGAAAGTCGAATTCGTGGATCTGCGA
ACACTGGGCTATAGCCAGCAGCAGCAGGAGAAGATCAAACCCAAGGTGAGGTC
CACAGTCGCACAGCACCATGAAGCCCTGGTGGGCCACGGGTTCACTCACGCTCA
TATTGTCGCACTGTCTCAGCATCCAGCCGCTCTGGGAACCGTGGCAGTCACATAC
CAGCACATCATTACTGCCCTGCCCGAGGCTACCCATGAAGACATCGTGGGAGTC
GGCAAACAGTGGAGCGGCGCACGGGCCCTGGAGGCTCTGCTGACCGACGCAGG
GGAACTGAGAGGACCCCCTCTGCAGCTGGATACAGGGCAGCTGGTGAAGATTGC
TAAGAGGGGAGGGGTGACAGCAATGGAAGCCGTCCACGCAAGCAGGAACGCAC
TGACAGGGGCCCCCCTGAACCTGACCCCGGACCAAGTGGTGGCTATCGCCAGCA
ACATTGGCGGCAAGCAAGCGCTCGAAACGGTGCAGCGGCTGTTGCCGGTGCTGT
GCCAGGACCATGGCCTGACCCCGGACCAAGTGGTGGCTATCGCCAGCAACAATG
GCGGCAAGCAAGCGCTCGAAACGGTGCAGCGGCTGTTGCCGGTGCTGTGCCAGG
ACCATGGCCTGACCCCGGACCAAGTGGTGGCTATCGCCAGCAACATTGGCGGCA
AGCAAGCGCTCGAAACGGTGCAGCGGCTGTTGCCGGTGCTGTGCCAGGACCATG
GCCTGACCCCGGACCAAGTGGTGGCTATCGCCAGCCACGATGGCGGCAAGCAAG
CGCTCGAAACGGTGCAGCGGCTGTTGCCGGTGCTGTGCCAGGACCATGGCCTGA
CTCCGGACCAAGTGGTGGCTATCGCCAGCAACATTGGCGGCAAGCAAGCGCTCG
AAACGGTGCAGCGGCTGTTGCCGGTGCTGTGCCAGGACCATGGCCTGACCCCGG
ACCAAGTGGTGGCTATCGCCAGCAACGGTGGCGGCAAGCAAGCGCTCGAAACG
GTGCAGCGGCTGTTGCCGGTGCTGTGCCAGGACCATGGCCTGACTCCGGACCAA
GTGGTGGCTATCGCCAGCAACGGTGGCGGCAAGCAAGCGCTCGAAACGGTGCAG
CGGCTGTTGCCGGTGCTGTGCCAGGACCATGGCCTGACCCCGGACCAAGTGGTG
GCTATCGCCAGCAACAATGGCGGCAAGCAAGCGCTCGAAACGGTGCAGCGGCTG
TTGCCGGTGCTGTGCCAGGACCATGGCCTGACCCCGGACCAAGTGGTGGCTATC
GCCAGCAACATTGGCGGCAAGCAAGCGCTCGAAACGGTGCAGCGGCTGTTGCCG
GTGCTGTGCCAGGACCATGGCCTGACTCCGGACCAAGTGGTGGCTATCGCCAGC
AACAATGGCGGCAAGCAAGCGCTCGAAACGGTGCAGCGGCTGTTGCCGGTGCTG
TGCCAGGACCATGGCCTGACCCCGGACCAAGTGGTGGCTATCGCCAGCAACATT
GGCGGCAAGCAAGCGCTCGAAACGGTGCAGCGGCTGTTGCCGGTGCTGTGCCAG
GACCATGGCCTGACCCCGGACCAAGTGGTGGCTATCGCCAGCAACGGTGGCGGC
AAGCAAGCGCTCGAAACGGTGCAGCGGCTGTTGCCGGTGCTGTGCCAGGACCAT
GGCCTGACCCCGGACCAAGTGGTGGCTATCGCCAGCCACGATGGCGGCAAGCAA
GCGCTCGAAACGGTGCAGCGGCTGTTGCCGGTGCTGTGCCAGGACCATGGCCTG
ACCCCGGACCAAGTGGTGGCTATCGCCAGCAACGGTGGCGGCAAGCAAGCGCTC
GAAACGGTGCAGCGGCTGTTGCCGGTGCTGTGCCAGGACCATGGCCTGACCCCG
GACCAAGTGGTGGCTATCGCCAGCAACGGTGGCGGCAAGCAAGCGCTCGAAAC
GGTGCAGCGGCTGTTGCCGGTGCTGTGCCAGGACCATGGCCTGACTCCGGACCA
AGTGGTGGCTATCGCCAGCCACGATGGCGGCAAGCAAGCGCTCGAAACGGTGCA
GCGGCTGTTGCCGGTGCTGTGCCAGGACCATGGCCTGACCCCGGACCAAGTGGT
GGCTATCGCCAGCCACGATGGCGGCAAGCAAGCGCTCGAAACGGTGCAGCGGCT
GTTGCCGGTGCTGTGCCAGGACCATGGCCTGACCCCGGACCAAGTGGTGGCTAT
CGCCAGCAACGGTGGCGGCAAGCAAGCGCTCGAAAGCATTGTGGCCCAGCTGAG
CCGGCCTGATCCGGCGTTGGCCGCGTTGACCAACGACCACCTGGTCGCTCTGGCT
TGCCTGGGAGGACGCCCTGCTATGGACGCTGTGAAGAAAGGACTGCCCCACGCA
CCCGAACTGATTAGACGGGTGAACCGGAGAATCGGCGAGAGAACATCCCATAG
GGTGGCAATCTCTAGAACTCAGCTGGTCAAGAGTGAACTGGAGGAAAAGAAATC
AGAGCTGCGCCACAAGCTGAAATACGTGCCTCATGAGTATATCGAACTGATCGA
GATTGCTCGCAATTCAACCCAGGACCGGATCCTGGAAATGAAAGTGATGGAGTT
CTTTATGAAAGTCTACGGATATCGGGGGAAACACCTGGGAGGGAGCAGAAAGCC
AGATGGGGCCATCTACACAGTGGGATCCCCCATCGACTATGGCGTGATTGTCGAT
ACTAAAGCCTACAGCGGAGGCTATAACCTGCCTATCGGCCAGGCTGACGAGATG
CAGAGATACGTGGAGGAAAACCAGACCCGCAATAAGCATATTAACCCCAATGA
ATGGTGGAAAGTGTATCCTAGCTCCGTCACAGAGTTCAAGTTTCTGTTCGTGAGC
GGACACTTTAAGGGCAACTACAAAGCACAGCTGACTAGGCTGAATCATATCACC
AACTGCAATGGAGCCGTGCTGTCTGTCGAGGAACTGCTGATCGGGGGAGAGATG
ATTAAGGCTGGCACACTGACTCTGGAGGAAGTGAGGCGCAAGTTCAACAATGGG
GAAATCAACTTCTAA
SEQ ID NO: 3 CRISPR GUIDE sequence
GGTATGTTCTGCTGAACCGC
SEQ ID NO: 4 WAS gene sequence
AGCAGAAGGGGTTCTGAACCTAGGTTCAGGAGAGAGGCTTTGAACCTGCACGTG
TGGGAAGCCATGGAAGTTTCCAGGAAGGACTGCAGGTCCCAACTGGAGATGTGC
CGTTCCTCCTTCAGGTACCTGGGAATGTCAGTCACACCCCAGACCTGCTCAGCTC
CCCCAAACTGCTGTTCCTGTATCTGAGAGCTTCAAGTCTCCAAATGGCCTACCTC
ATACATGGGGAAACTGAGGCCTGGGGAGGCCGGGGACTGAGCTAGCATTCACTT
GTGGAAATAGTCTGGCATCATCTGGAGAAGTTAGAGACATGCAAACCCTACAGC
CCTCAGATTCCCGTCTGAGAGTCTGCATGCCTATGTGGACCAGGAGATGTGTGCG
GGAGTGAACACTGCAGTGTTGCTCCCAACAGCAAGAACCAGAAGCAGCCCAAA
GGGCTGTTACAGGAGAATATGGACACCCAGGCTGCACATGCACACCATGGAATG
CTGTATGGCAGTGGAAATAAATGAACAGCTACCACTATAGGCAAACAGGAATCA
CAGCAACAGCCAAGAGTGAAGGCGTGGAGGGACGAGACCATGCACTCACACCT
GGCCTGCCTGGCTCGCACTCCGGGCAAAGGGGTCAGAACAGTGACTGGCACACA
CGTTAAGTGCTATGTGAGTGTTAAGATAAAACTAGGATGTCCAGTGGGGAGAAA
GCAAGCCTTTGAAGATTATGTGCTTTTACAAACTTCAAGTGCAATGAAAACTAAA
CAAGATGTTGTTCAGGCATTCATATATGATATAAAGTTCCTTTCTTTAAAAAAGG
GATGGGCTGGGCACGGTGGCTCACGCCTGTAATTCTAATACTTTGGGAGGCCGA
GGCAGGTGGATCACGAGGTCGAGAAATCGAGACCATCCTGGCCAACATGGTGAA
ACCCTGTCTCTACTAAAAATACAAAAAAATTAGCTGGGCGTGGTGGCGTGTGCCT
GTAGTCCCAGCTACTTGGGAGGCTGAGGCAGGAGAGTCACTTGAACCCGGGAGG
CAAAGGTTGCAGTGAGCCGAGATCGTGCCACCGCACTCCAGCCTGGCGACAGAG
TGAGACTCCATCTCAAAAAAAAAAAGAAAAAAAAAAGTATGACAAGCAGAAAG
TAATTTGGGAGCTGCGGGGAGGCAAGGGTAAGGGATGGGGAAGTGGACCAGAG
GCATATGCGTCATTGGCAGTGTCTAAGCACTCACGATAGGCGTGGATCACAGGG
GCTCGCTCTGTAATTAAAAGGAAAAGGGTTTTTGTTGTGTTGTTGTTGTTGCTGTT
TTTGAGACAAGGGTCTTGCTCTGTCATCATCCAGGCTGGAGTGCAGTGGTGCAGT
CTCAGCTCACTGCAACCTCCGCCTCCTGGGTTCAAGCGATTCTCCTGCCTCAGCC
TCCTGAGCAGCTAGGACTACAGGTGTGTGCCACCATGCCTGGCTAATTTTTGTAT
TTTTTAGTGGAAATGGGGTTTTGCCATGTTGCCCAGGCTCGTCTTGAACTCCTGA
CCTCAAGTGATCCACTCGTCTCGGCCTCCCAAAGTGCTGGGATTACAGGTGTGAG
CTATTGTCCCCAGCCAAAAGGAAAAGTTTTACTGTAGTAACCCTTCCGGACTAGG
GACCTCGGGCCTCAGCCTCAGGCTACCTAGGTGCTTTAGAAAGGAGGCCACCCA
GGCCCATGACTACTCCTTGCCACAGGGAGCCCTGCACACAGATGTGCTAAGCTCT
CGCTGCCAGCCAGAGGGAGGAGGGTCTGAGCCAGTCAGAAGGAGATGGGCCCC
AGAGAGTAAGAAAGGGGGAGGAGGACCCAAGCTGATCCAAAAGGTGGGTCTAA
GCAGTCAAGTGGAGGAGGGTTCCAATCTGATGGCGGAGGGCCCAAGCTCAGCCT
AACGAGGAGGCCAGGCCCACCAAGGGGCCCCTGGAGGACTTGTTTCCCTTGTCC
CTTGTGGTTTTTTGCATTTCCTGTTCCCTTGCTGCTCATTGCGGAAGTTCCTCTTCT
TACCCTGCACCCAGAGCCTCGCCAGAGAAGACAAGGGCAGAAAGCACCATGAG
TGGGGGCCCAATGGGAGGAAGGCCCGGGGGCCGAGGAGCACCAGCGGTTCAGC
AGAACATACCCTCCACCCTCCTCCAGGACCACGAGAACCAGCGACTCTTTGAGA
TGCTTGGACGAAAATGCTTGGTGAGCTGGGGATCTCCTGCCCCCGCCCCGTCCCC
ACCGTTTCTTCCTCTTCCTCTCCTCCTTCTCTCTCTTCCCCTCCTCCCGCTCCTCCT
TTCCCTCTCCATCATCTCCTCTCCTAGAATTTCCCGTCATAATCCACCCTTCCCAG
GAAGATCTCAATGTCTACTTGCCTTCCCTCTGGCTGCAGCTCTTCCTTTGGGCCCA
TGACTGTCATGAGGCAGGAAGGACCAGGTCTGGCTCCAAGACCTTGTGGCTACC
CC
TGACCAGACTCCACTGACCCCTGCTTTCCTCTCCCAGACGCTGGCCACTGCAGTT
GTTCAGCTGTACCTGGCGCTGCCCCCTGGAGCTGAGCACTGGACCAAGGAGCAT
TGTGGGGCTGTGTGCTTCGTGAAGGATAACCCCCAGAAGTCCTACTTCATCCGCC
TTTACGGCCTTCAGGTGACCCCCCCACCCCCGACTGGACTTGCAAGCCAGTTCTC
AACCCGCAAACCCAGATCTGTGTCCATATGTGTCCATAGCTTCAAGACCTCAGAC
CTGATCAGTGAATCCCTGAGCCCCAGAACCAAAGACTCATCCAGATGGCAAACT
CTGACTTGCCTTTCTAAGTCTGCAATGACTGGCCCCAGTCTCCGTATCAAGATCT
CTAAAGCCCCCAGTATTAGTCTGCTGCCTAAGCCTAATCTTTTCCACAAATTCCA
ATAAATGAGCACTGTATTTGTACCTGAACCTCAAATCTATTCTAAACTCAACATT
TTGCATCCCAGGAATCTCTCATCAAAACTCCTGAACCCCAGATGTTTGCCAAGCT
CCTAAGTCATAAATCTGTTCAACAAACCCCAAAGTTGAATATTCCATTGATCCTT
GAACTCCAAATCTGTCCTTCTAAATCCACAGCACAGACCCCAGAGTTCCCATATT
AAAATTCCTGAACACTCAAATACCGAGGTAGTTCTTAAGCAAAAAGTCTTTTCCA
CAATCCCCTGACCTGAACTTTCTAGGTTTAAGCCCCAAATTCATCCTTTTAAACC
CATAAAGATGGACCCAGCATAACTTCCAGATCCCAAGGCTATCAAATATCCACC
AAACTCCTAAACCATAACTCTCTCCACAAACCCCAAATTGCACTTACTTTAGCTG
GACTCCCCGCGAAACTCCCAAGTCTATGTGTCTGAACTTCAAATCTCAACTCCAA
CCCCCAAATACTAGAATCCTACCTGTCATGAATTGGGGCTGGGGTGGTGGGGGA
GGGCATGGATTGAATCTGTGAATGAGCCTCAACTTCCTAAGACTAGAGTCCTAA
ATTATGAAATTCAAGCCCCCAAGTCCCAGATCTAGGGCCCCAAACCCCAAATCC
AAACCTCTCACAAAAGTGTATGGCTCCCAGACTATACCCCACAATCCACACCCTT
AGACACCAACTCTCTGGTGCTGAGCTGAAAATCTCCAAACCAGACTATGAGGCT
CCCAAATCCAGACACCCTGCTCCCTGCCCAGCTAACAAAAGCCTGCCACCCCCG
GCG
TGCCTCAGTGCCACTGTGCCTCCCACCCTACACCTCTCCAGGCTGGTCGGCTGCT
CTGGGAACAGGAGCTGTACTCACAGCTTGTCTACTCCACCCCCACCCCCTTCTTC
CACACCTTCGCTGGAGATGTAAGTGATCAACCAGCCCTCGGGCCTCACTTGGGGT
GTGGAGAGGAGATGGGAAAGTTGCGGGGGACCTGGGAGGCGGCTGACCCCAAG
GTATGTGCAGGACTGCCAAGCGGGGCTGAACTTTGCAGACGAGGACGAGGCCCA
GGCCTTCCGGGCCCTCGTGCAGGAGAAGATACAAAAAAGGAATCAGAGGCAAA
GTGGAGGTGAGGAGGCCACAGGGGAGGAAAGGAAGTTGGGCAGAGGTGAGTGC
AAGCCTGGGGAACTAGAAAAGTCCCCTCTCATGGTCCTGGCTCCCAATCCATCTA
TCCACAGACAGACGCCAGCTACCCCCACCACCAACACCAGCCAATGAAGGTGAG
TCCTCTAGTGCAAGTAGGGGTAATAAGGGGCTAGCCCAGGAACCTGTGGCAGGG
CTGTGATAACTCTCTACACATTCCATCTTCCCAGAGAGAAGAGGAGGGCTCCCAC
CCCTGCCCCTGCATCCAGGTGGAGACCAAGGAGGTGCGTGCTGATTCTTCCCTGT
GTCTCTGGATGGATGGGTAAGAGTGGATGGAGGAATGAGGAGTTGGATGGGTGC
GTAAGTGGGTGAATGGATAGGTAGATTGATAGGTATGTGGATGGACGAGCAGGT
GCATGGATGTGTGGACTGATGGATGGGTGGATGGATTGGCGGTAGATGGCTGAG
TAGAGGGATGAATTGATGGGAGGATGAAAGTCTAAGTAGATAGATGCATAGGTG
AATGGGTATGTGGATAAATGAATGAAAAGGTAGATGGATGACTGAGTAAATTAA
TCAATGAGTGAATGAATGAACAGTGAATAAATGACTAAATGACAAGTTTCAGTC
AGTGAAGAAAGCATGATTGAATGAATAAATGAGTAAATGAATATTTTAACAAAT
TCATTAGTCAATGAGCCAGTGAATGATAAAGCATGAGGGAATGAAAACATGAAT
GAATCAGTGAATGTATGAATGGTTTGTGGGATCCACCCACTTCTCCATAGACCCT
ACTTGAACCCTTCACCCACTACCTCCATGACCATCCAACACACACACAGATTTCC
CTCAAGGCTTCCGTTTCTTGCCCCTGTGCTTTGGTTGGTTGGTAAGTGGGTCAATG
AGCCAACCACCCTATTTTCCCCACAGGCCCTCCAGTGGGTCCGCTCTCCCTGGGG
CTGGCGACAGTGGACATCCAGAACCCTGACATCACGAGTTCACGATACCGTGGG
CTCCCAGCACCTGGACCTAGCCCAGCTGATAAGAAACGCTCAGGGAAGAAGAAG
ATCAGCAAAGCTGATATTGGTGCACCCAGTGGATTCAAGTGAGAGCCACTCCCC
AGTGGACCCACAGATTCCTGGGGGCAGAGGGGCACATGAACAAGTGGACAGCT
GAGTGAATGGAAGGATGGGCAGATGGGCAGATGGCTGGGTGGCTGAGTGGGTA
AATGGGTGGTTGGATAGGTAGGTGCAGGGCTGGGTCTAGGGAGAGGTAAATAAG
GCACCAAGGGTACAAAATTTAAGGAGGCACTCACTCTCAGAGGCATGCAACTGT
AATTCCTGACTCTCAGAGTGAGTGACTCACTTAAATTTTGCACCCTAGGCACCTT
ACTTGCCTCACCCTGGGCCCACTCTGGGTGGGCTGTTAGGAGAGCAGGTGGGTG
GGCAGGTGAACAAATGGATAGATAGATGAGGTAGATGATGGATGAGAAGGGCT
GGTGGGTAGGTGGGTGAGTGGATGGGTGGATGGATGGATAAATGAATGGATGA
ATGAATGGGTTGAAGAATGAATGGATAAGTGGTTGGATGGACAAGTTTATGGGT
GGATGGGTTGATGGGAGGTGCGTGGATAGATAGATGGGTGAGTGGATAGGTGTG
TGGACAGATTGATATGCAGGCTGATTGGCTCACAGACAAGGTGGATGGGGATGG
ACAGGTGGACAGATACGTGGATGAATGGACAGTTCAATGGATAAGTGAACAGA
AGTGTGTGGTTGCATGGGTAGAAAAATGAGTGGATGGATAGATGGAAAGGTGGG
CACATGGGTAGGTGGATGGGTGGATGGACAAGTGTGTGTGAGGACAGACTGGTG
GACAAATGGGTGAACAGACATATGTGGGCAGATAGTTGCAGAGACAGATGTATG
GACAGATCAGTAGTCCAACAGATGAATGTGAATGAATAGGTGGACAAATGCATG
GGATAGATGGGGAAAGAGGGATGGGTGGATGGATCAGCACCACAAACTATGGA
G
CCCTTCTAATTCCATAACTCCTGCCTATACTCATTCACTCATTCAGTCTCATTCAT
TAATTCTGGCCCCTCAGAGTCTCTTTGGGCAGGAGAGGGCAAGAGGGTTTCACTA
TGAAGGGAGGGAAGGAAGGGCAGTGAGGATTCACTGGAGTCTCTTCACCTCTCC
CAGGCATGTCAGCCACGTGGGGTGGGACCCCCAGAATGGATTTGACGTGAGTAA
CTTCAGAGTCTCTTGGACTCCACTAAACTTCCACCCACCCTTCCAAAGACCACTG
CTGAGACCCCACCCCCAGATCGTGCCCTTCCCACACCCCTCTCAGATCCCTTGCT
GGGATGGACCCAACGACAATCCATGTCGCTTGTCTCCTCGCCTTATTCCTCTACT
CCTGCCCCTGGCCTTTTTCCTCCTGGGCAGGTGAACAACCTCGACCCAGATCTGC
GGAGTCTGTTCTCCAGGGCAGGAATCAGCGAGGCCCAGCTCACCGACGCCGAGA
CCTCTAAACTTATCTACGACTTCATTGAGGACCAGGGTGGGCTGGAGGCTGTGCG
GCAGGAGATGAGGCGCCAGGGTGAGACCCTGCTTCCATACGCTCCCTTCTCTAG
CCCAAGCAGCTCATAGCTAAGATACGCACTAAGTCACTCAGTCCTTATGGGAGC
ACCTATACTGCTTCAGTCAGGAGTTGGTCAGTGGGGGTACCCATTTTACAAATGA
GCAAAACTGAGGCTCAGAAGAAATCAATGAGAGTTACAGCTATGTGTTATACCC
CCTCCACAGAGCCACTTCCGCCGCCCCCACCGCCATCTCGAGGAGGGAACCAGC
TCCCCCGGCCCCCTATTGTGGGGGGTAACAAGGGTCGTTCTGGTCCACTGCCCCC
TGTACCTTTGGGGATTGCCCCACCCCCACCAACACCCCGGGGACCCCCACCCCCA
GGCCGAGGGGGCCCTCCACCACCACCCCCTCCAGCTACTGGACGTTCTGGACCA
CTGCCCCCTCCACCCCCTGGAGCTGGTGGGCCACCCATGCCACCACCACCGCCAC
CACCGCCACCGCCGCCCAGCTCCGGGAATGGACCAGCCCCTCCCCCACTCCCTCC
TGCTCTGGTGCCTGCCGGGGGCCTGGCCCCTGGTGGGGGTCGGGGAGCGCTTTTG
GATCAAATCCGGCAGGGAATTCAGCTGAACAAGGTGAGGACAGGCAGGATGGA
GGATTGGGGGTCTAGGACTCTGGGGTGTCCCGTCTAAGTCAGGATACTGGGGGG
CTGAGGCCAGGACTGAGGAGAGTGCCAGGCCTTAGGGATTCAGTGATAGGGTTG
AAAGGTTGGTGGGAAGCCTTGAAGGGGACTGGAGTGTGTGGGAGAGAAAATATT
GATGGAGGGGCGGGGAGAAATGCTCCTTTCCCAGGCCCTAAGCCCTCTGTGCTG
ATCCCTGCCTGCTGCAGACCCCTGGGGCCCCAGAGAGCTCAGCGCTGCAGCCA
CCACCTCAGAGCTCAGAGGGACTGGTGGGGGCCCTGATGCACGTGATGCAGAAG
AGAAGCAGAGCCATCCACTCCTCCGGTGAGCTGATCCTGCCGGGGCCTCAAACC
TGGCTCCCAGGGCTAGCACTGGCCTCAAAACAATCCCAGCAGTCACCACCAATA
GTGACATCAGCCCCATCTGTTTGACAGCATTAACATGAATCTTGTGTCAGCCTCG
TTTTTGACAATGTTAACATTAAGTCATTATGTGACAATAATATAATTAACTCCAA
CTTTGACAGTAATATTAACATTAATGCCAGGGTGTGTCCACAATATTAATGTCAT
TCCCACATGTTCAGTACTACTAACATCAGCTGGCCGGGCGCGGTGGCTCATGCCT
GTAATCCAGGAACTTTGGGAGGCTAAGGCAGGAGGATCACTTGAGCCCAGGAGT
TCGAGACCAGCCTGGGCAATATAGTGAGACCTCGTTTCCATAAAAACTAAATTC
AAAAAAAGTAGTCAAGCATAGTGGTGTGTGCCTGTGGTCCCAGCTACTTGGGAG
GCTGAGGTGGGAGGATTGCTTGACCCTGGGAGGTCAAGGCAGCAGTGATCCATG
ATTGTGCCACTGCACTCCAGCCTGGGTGACAGAGATCCTATCTCAAAAAAAAAA
AAAATTAACCCATTATGTGATGACAATATTATGAAGAACACTATTGTTGACAATA
TTAATTTTAATTCCATGTATTAACAGATTTACATTAATTCATTATGACGTAACCTA
ATCTAATCTTTTAAAAAATTTTTTTGAAACAGGGTCTCGCTCTGTGTCCCAGGCTG
GAGTACAGTGGTGCAATCATGGCTCAGTGCAGCCTCAACCTCCCAGGCTCAAGC
GATCCTCCCGCCTCAGCTCCCAAAGTAGCTGGGACTACAGGCGTGTGCCACCAT
ACCTGGCTAATTTTTGGTGTTTTTTTGGTAGTGATGAGCTCTCACTACCAAGCTCT
CACTACTCTCATGTTGCCCAGGCTGCTCTGCAACTCCAGGGCTCAAGCGATCTGC
CCCGCCTCAGCCTCCCAGAGTGCTGGGATTACAGGCATGAGCCACCAGGCCTGG
CTGTTAACCTAATCTTTTTATAATAATGTTACTATTACTCTCTTAATCTGTCAGCA
ATACTGTCACTAATCCATTATATGATGCAAATATTAGTATCAACCTACTATAGGA
ACTTCATCTTTCGACAATGATTTTTTTTTTTCTTTTGAGACGGAGTCTTGCTCTGTC
ACCCAGGCTGGAGTGCAGTGGCGCGATCTTGGCTAACTGCAGACTCTGCCTCCTG
GGTTCAAGCGATTCTCCTGCCTCAGCCTTCCGAGTAGCTGGGACTACAGGCACGC
CACTACGCCCAGCTAATTTTGATGTTATTGTCATTAACCCCATTATGTGTCAAAA
ATATTAGCGTTAACCAGACAGGAAGCAATAATATATTATCACACCTTTGCTAATA
TTATTTAAATTCACCCTATTATGTGATAAATAGGTTAACATTAACCCTTTGTTTGA
CAATATCTCGACTAACCACATTTTTGACAGCATAAACTTCAACTCCAACTAGAAC
TCAGACCCCAACTATAATCCCTTTCTTGTCCCAAATGGAAACTCTAACTTGCCCT
CCTCTAGCATGAGACCTCAGAACCCCAGGGTCCAGTCCTCACCTCCCAGGCCCTA
TGAAGCCCCCCACCAACCTCCCAGGGCATCTTATCTTTCTCTTTCCCTCCAGACG
AAGGGGAGGACCAGGCTGGCGATGAAGATGAAGATGATGAATGGGATGACTGA
GTGGCTGAGTTACTTGCTGCCCTGTGCTCCTCCCCGCAGGACATGGCTCCCCCTC
CACCTGCTCTGTGCCCACCCTCCACTCTCCTCTTCCAGGCCCCCAACCCCCCATTT
CTTCCCCACCAACCCCTCCAATGCTGTTATCCCTGCCTGGTCCTCACACTCACCCA
ACAATCCCAAGGCCCTTTTTATACAAAAATTCTCAGTTCTCTTCACTCAAGGAT
TTTTAAAGAAAAATAAAAGAATTGTCTTTCTGTCTCTCTATAAA
SEQ ID NO: 5 AAV #1201
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTGTATGACAAGCAGAAAGTAATTTGGGAGCTG
CGGGGAGGCAAGGGTAAGGGATGGGGAAGTGGACCAGAGGCATATGCGTCATT
GGCAGTGTCTAAGCACTCACGATAGGCGTGGATCACAGGGGCTCGCTCTGTAAT
TAAAAGGAAAAGGGTTTTTGTTGTGTTGTTGTTGTTGCTGTTTTTGAGACAAGGG
TCTTGCTCTGTCATCATCCAGGCTGGAGTGCAGTGGTGCAGTCTCAGCTCACTGC
AACCTCCGCCTCCTGGGTTCAAGCGATTCTCCTGCCTCAGCCTCCTGAGCAGCTA
GGACTACAGGTGTGTGCCACCATGCCTGGCTAATTTTTGTATTTTTTAGTGGAAA
TGGGGTTTTGCCATGTTGCCCAGGCTCGTCTTGAACTCCTGACCTCAAGTGATCC
ACTCGTCTCGGCCTCCCAAAGTGCTGGGATTACAGGTGTGAGCTATTGTCCCCAG
CCAAAAGGAAAAGTTTTACTGTAGTAACCCTTCCGGACTAGGGACCTCGGGCCT
CAGCCTCAGGCTACCTAGGTGCTTTAGAAAGGAGGCCACCCAGGCCCATGACTA
CTCCTTGCCACAGGGAGCCCTGCACACAGATGTGCTAAGCTCTCGCTGCCAGCCA
GAGGGAGGAGGGTCTGAGCCAGTCAGAAGGAGATGGGCCCCAGAGAGTAAGAA
AGGGGGAGGAGGACCCAAGCTGATCCAAAAGGTGGGTCTAAGCAGTCAAGTGG
AGGAGGGTTCCAATCTGATGGCGGAGGGCCCAAGCTCAGCCTAACGAGGAGGCC
AGGCCCACCAAGGGGCCCCTGGAGGACTTGTTTCCCTTGTCCCTTGTGGTTTTTT
GCATTTCCTGTTCCCTTGCTGCTCATTGCGGAAGTTCCTCTTCTTACCCTGCACCC
AGAGCCTCGCCAGAGAAGACAAGGGCAGAAAGCACCATGAGTGGGGGCCCAAT
GGGAGGAAGGCCCGGGGGCCGAGGAGCACGAACAGAGAAACAGGAGAATATG
GGCCAAACAGGATATCTGTGGTAAGCAGTTCCTGCCCCGGCTCAGGGCCAAGAA
CAGTTGGAACAGCAGAATATGGGCCAAACAGGATATCTGTGGTAAGCAGTTCCT
GCCCCGGCTCAGGGCCAAGAACAGATGGTCCCCAGATGCGGTCCCGCCCTCAGC
AGTTTCTAGAGAACCATCAGATGTTTCCAGGGTGCCCCAAGGACCTGAAATGAC
CCTGTGCCTTATTTGAACTAACCAATCAGTTCGCTTCTCGCTTCTGTTCGCGCGCT
TCTGCTCCCCGAGCTCTATATAAGCAGAGCTCGTTTAGTGAACCGTCAGATCGCC
TGGAGACGCCATCCACGCTGTTTTGACTTCCATAGAAGGATCTCGAGGCCACCAT
GGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAG
CCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACA
ACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCG
ACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGA
CGAGCTGTACAAGTAAACTAGTGTCGACTGCTTTATTTGTGAAATTTGTGATGCT
ATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAACAGC
GGTTCAGCAGAACATACCCTCCACCCTCCTCCAGGACCACGAGAACCAGCGACT
CTTTGAGATGCTTGGACGAAAATGCTTGGTGAGCTGGGGATCTCCTGCCCCCGCC
CCGTCCCCACCGTTTCTTCCTCTTCCTCTCCTCCTTCTCTCTCTTCCCCTCCTCCCG
CTCCTCCTTTCCCTCTCCATCATCTCCTCTCCTAGAATTTCCCGTCATAATCCACC
CTTCCCAGGAAGATCTCAATGTCTACTTGCCTTCCCTCTGGCTGCAGCTCTTCCTT
TGGGCCCATGACTGTCATGAGGCAGGAAGGACCAGGTCTGGCTCCAAGACCTTG
TGGCTACCCCTGACCAGACTCCACTGACCCCTGCTTTCCTCTCCCAGACGCTGGC
CACTGCAGTTGTTCAGCTGTACCTGGCGCTGCCCCCTGGAGCTGAGCACTGGACC
AAGGAGCATTGTGGGGCTGTGTGCTTCGTGAAGGATAACCCCCAGAAGTCCTAC
TTCATCCGCCTTTACGGCCTTCAGGTGACCCCCCCACCCCCGACTGGACTTGCAA
GCCAGTTCTCAACCCGCAAACCCAGATCTGTGTCCATATGTGTCCATAGCTTCAA
GACCTCAGACCTGATCAGTGAATCCCTGAGCCCCAGAACCAAAGACTCATCCAG
ATGGCAAACTCTGACTTGCCTTTCTAAGTCTGCAATGACTGGCCCCAGTCTCCGT
ATCAAGATCTCTAAAGCCCCCAGTATTAGTCTGCTGCCTAAGCCTAATCTTTTCC
ACAAATTCCAATAAATGAGCACTGTATTTGTACCTGAACCTCAAATCTATTCTAA
ACTCAACATTTTGCATCCCAGGAATCTCTCATCAAAACTCCTGAACCCCAGATGT
TTGCCAAGCTCCTAAGTCATAAATCTGTTCAACAAACCCCAAAGTTGAATATTCC
ATTGATCCTTGAACTCCAAATCTGTCCTTCTAAATCCACAGCACAGACCCCAGAG
TTCCCATCTAGAGCATGGCTACGTAGATAAGTAGCATGGCGGGTTAATCATTAAC
TACAAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGG
CCTCAGTGAGCGAGCGAGCGCGCCAGCTGGCGTAATAGCGAAGAGGCCCGCACC
GATCGCCCTTCCCAACAGTTGCGCAGCCTGAATGGCGAATGGCGATTCCGTTGCA
ATGGCTGGCGGTAATATTGTTCTGGATATTACCAGCAAGGCCGATAGTTTGAGTT
CTTCTACTCAGGCAAGTGATGTTATTACTAATCAAAGAAGTATTGCGACAACGGT
TAATTTGCGTGATGGACAGACTCTTTTACTCGGTGGCCTCACTGATTATAAAAAC
ACTTCTCAGGATTCTGGCGTACCGTTCCTGTCTAAAATCCCTTTAATCGGCCTCCT
GTTTAGCTCCCGCTCTGATTCTAACGAGGAAAGCACGTTATACGTGCTCGTCAAA
GCAACCATAGTACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGG
AACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTT
TAACAAAATATTAACGTTTACAATTTAAATATTTGCTTATACAATCTTCCTGTTTT
TGGGGCTTTTCTGATTATCAACCGGGGTACATATGATTGACATGCTAGTTTTACG
ATTACCGTTCATCGATTCTCTTGTTTGCTCCAGACTCTCAGGCAATGACCTGATAG
CCTTTGTAGAGACCTCTCAAAAATAGCTACCCTCTCCGGCATGAATTTATCAGCT
AGAACGGTTGAATATCATATTGATGGTGATTTGACTGTCTCCGGCCTTTCTCACC
CGTTTGAATCTTTACCTACACATTACTCAGGCATTGCATTTAAAATATATGAGGG
TTCTAAAAATTTTTATCCTTGCGTTGAAATAAAGGCTTCTCCCGCAAAAGTATTA
CAGGGTCATAATGTTTTTGGTACAACCGATTTAGCTTTATGCTCTGAGGCTTTATT
GCTTAATTTTGCTAATTCTTTGCCTTGCCTGTATGATTTATTGGATGTTGGAATCG
CCTGATGCGGTATTTTCTCCTTACGCATCTGTGCGGTATTTCACACCGCATATGGT
GCACTCTCAGTACAATCTGCTCTGATGCCGCATAGTTAAGCCAGCCCCGACACCC
GCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCAT
CACCGAAACGCGCGAGACGAAAGGGCCTCGTGATACGCCTATTTTTATAGGTTA
ATGTCATGATAATAATGGTTTCTTAGACGTCAGGTGGCACTTTTCGGGGAAATGT
GCGCGGAACCCCTATTTGTTTATTTTTCTAAATACATTCAAATATGTATCCGCTCA
TGAGACAATAACCCTGATAAATGCTTCAATAATATTGAAAAAGGAAGAGTATGA
GTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGG
GTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATG
TGGCGCGGTATTATCCCGTATTGACGCCGGGCAAGAGCAACTCGGTCGCCGCAT
ACACTATTCTCAGAATGACTTGGTTGAGTACTCACCAGTCACAGAAAAGCATCTT
ACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGG
AGCTGAATGAAGCCATACCAAACGACGAGCGTGACACCACGATGCCTGTAGCAA
TGGCAACAACGTTGCGCAAACTATTAACTGGCGAACTACTTACTCTAGCTTCCCG
GCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGT
GGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCA
GCAGAGCGCAGATACCAAATACTGTCCTTCTAGTGTAGCCGTAGTTAGGCCACC
ACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGCG
CCCAATACGCAAACCGCCTCTCCCCGCGCGTTGCCGATTCATTAATG
SEQ ID NO: 6 AAV #1244
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTGTATGACAAGCAGAAAGTAATTTGGGAGCTG
CGGGGAGGCAAGGGTAAGGGATGGGGAAGTGGACCAGAGGCATATGCGTCATT
GGCAGTGTCTAAGCACTCACGATAGGCGTGGATCACAGGGGCTCGCTCTGTAAT
TAAAAGGAAAAGGGTTTTTGTTGTGTTGTTGTTGTTGCTGTTTTTGAGACAAGGG
TCTTGCTCTGTCATCATCCAGGCTGGAGTGCAGTGGTGCAGTCTCAGCTCACTGC
AACCTCCGCCTCCTGGGTTCAAGCGATTCTCCTGCCTCAGCCTCCTGAGCAGCTA
GGACTACAGGTGTGTGCCACCATGCCTGGCTAATTTTTGTATTTTTTAGTGGAAA
TGGGGTTTTGCCATGTTGCCCAGGCTCGTCTTGAACTCCTGACCTCAAGTGATCC
ACTCGTCTCGGCCTCCCAAAGTGCTGGGATTACAGGTGTGAGCTATTGTCCCCAG
CCAAAAGGAAAAGTTTTACTGTAGTAACCCTTCCGGACTAGGGACCTCGGGCCT
CAGCCTCAGGCTACCTAGGTGCTTTAGAAAGGAGGCCACCCAGGCCCATGACTA
CTCCTTGCCACAGGGAGCCCTGCACACAGATGTGCTAAGCTCTCGCTGCCAGCCA
GAGGGAGGAGGGTCTGAGCCAGTCAGAAGGAGATGGGCCCCAGAGAGTAAGAA
AGGGGGAGGAGGACCCAAGCTGATCCAAAAGGTGGGTCTAAGCAGTCAAGTGG
AGGAGGGTTCCAATCTGATGGCGGAGGGCCCAAGCTCAGCCTAACGAGGAGGCC
AGGCCCACCAAGGGGCCCCTGGAGGACTTGTTTCCCTTGTCCCTTGTGGTTTTTT
GCATTTCCTGTTCCCTTGCTGCTCATTGCGGAAGTTCCTCTTCTTACCCTGCACCC
AGAGCCTCGCCAGAGAAGACAAGGGCAGAAAGCACCATGAGTGGGGGCCCAAT
GGGAGGAAGGCCCGGGGGCCGAGGAGCACGAACAGAGAAACAGGAGAATATG
GGCCAAACAGGATATCTGTGGTAAGCAGTTCCTGCCCCGGCTCAGGGCCAAGAA
CAGTTGGAACAGCAGAATATGGGCCAAACAGGATATCTGTGGTAAGCAGTTCCT
GCCCCGGCTCAGGGCCAAGAACAGATGGTCCCCAGATGCGGTCCCGCCCTCAGC
AGTTTCTAGAGAACCATCAGATGTTTCCAGGGTGCCCCAAGGACCTGAAATGAC
CCTGTGCCTTATTTGAACTAACCAATCAGTTCGCTTCTCGCTTCTGTTCGCGCGCT
TCTGCTCCCCGAGCTCTATATAAGCAGAGCTCGTTTAGTGAACCGTCAGATCGCC
TGGAGACGCCATCCACGCTGTTTTGACTTCCATAGAAGGATCTCGAGGCCACCAT
GGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAG
CCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACA
ACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCG
ACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGA
CGAGCTGTACAAGTAAACTAGTGTCGACTGCTTTATTTGTGAAATTTGTGATGCT
ATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAACAGC
GGTTCAGCAGAACATACCCTCCACCCTCCTCCAGGACCACGAGAACCAGCGACT
CTTTGAGATGCTTGGACGAAAATGCTTGGTGAGCTGGGGATCTCCTGCCCCCGCC
CCGTCCCCACCGTTTCTTCCTCTTCCTCTCCTCCTTCTCTCTCTTCCCCTCCTCCCG
CTCCTCCTTTCCCTCTCCATCATCTCCACTCCTAGAATTTCCCGTCATAATCCACC
CTTCCCAGGAAGATCTCAATGTCTTCTTGCCTTCCCTCTGGCTGCAGCTCTTCCTT
TGGGCCCATGACTGTCATGAGGCAGGAAGGACCAGGTCTGGCTCCAAGACCTTG
TGGCTACCCCTGACCAGACTCCACTGACCCCTGCTTTCCTCTCCCAGACGCTGGC
CACTGCAGTTGTTCAGCTGTACCTGGCGCTGCCCCCTGGAGCTGAGCACTGGACC
AAGGAGCATTGTGGGGCTGTGTGCTTCGTGAAGGATAACCCCCAGAAGTCCTAC
TTCATCCGCCTTTACGGCCTTCAGGTGACCCCCCCACCCCCGACTGGACTTGCAA
GCCAGTTCTCAACCCGCAAACCCAGATCTGTGTCCATATGTGTCCATAGCTTCAA
GACCTCAGACCTGATCAGTGAATCCCTGAGCCCCAGAACCAAAGACTCATCCAG
ATGGCAAACTCTGACTTGCCTTTCTAAGTCTGCAATGACTGGCCCCAGTCTCCGT
ATCAAGATCTCTAAAGCCCCCAGTATTAGTCTGCTGCCTAAGCCTAATCTTTTCC
ACAAATTCCAATAAATGAGCACTGTATTTGTACCTGAACCTCAAATCTATTCTAA
ACTCAACATTTTGCATCCCAGGAATCTCTCATCAAAACTCCTGAACCCCAGATGT
TTGCCAAGCTCCTAAGTCATAAATCTGTTCAACAAACCCCAAAGTTGAATATTCC
ATTGATCCTTGAACTCCAAATCTGTCCTTCTAAATCCACAGCACAGACCCCAGAG
TTCCCATCTAGAGCATGGCTACGTAGATAAGTAGCATGGCGGGTTAATCATTAAC
TACAAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGG
CCTCAGTGAGCGAGCGAGCGCGCCAGCTGGCGTAATAGCGAAGAGGCCCGCACC
GATCGCCCTTCCCAACAGTTGCGCAGCCTGAATGGCGAATGGCGATTCCGTTGCA
ATGGCTGGCGGTAATATTGTTCTGGATATTACCAGCAAGGCCGATAGTTTGAGTT
CTTCTACTCAGGCAAGTGATGTTATTACTAATCAAAGAAGTATTGCGACAACGGT
TAATTTGCGTGATGGACAGACTCTTTTACTCGGTGGCCTCACTGATTATAAAAAC
ACTTCTCAGGATTCTGGCGTACCGTTCCTGTCTAAAATCCCTTTAATCGGCCTCCT
GTTTAGCTCCCGCTCTGATTCTAACGAGGAAAGCACGTTATACGTGCTCGTCAAA
GCAACCATAGTACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGG
AACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTT
TAACAAAATATTAACGTTTACAATTTAAATATTTGCTTATACAATCTTCCTGTTTT
TGGGGCTTTTCTGATTATCAACCGGGGTACATATGATTGACATGCTAGTTTTACG
ATTACCGTTCATCGATTCTCTTGTTTGCTCCAGACTCTCAGGCAATGACCTGATAG
CCTTTGTAGAGACCTCTCAAAAATAGCTACCCTCTCCGGCATGAATTTATCAGCT
AGAACGGTTGAATATCATATTGATGGTGATTTGACTGTCTCCGGCCTTTCTCACC
CGTTTGAATCTTTACCTACACATTACTCAGGCATTGCATTTAAAATATATGAGGG
TTCTAAAAATTTTTATCCTTGCGTTGAAATAAAGGCTTCTCCCGCAAAAGTATTA
CAGGGTCATAATGTTTTTGGTACAACCGATTTAGCTTTATGCTCTGAGGCTTTATT
GCTTAATTTTGCTAATTCTTTGCCTTGCCTGTATGATTTATTGGATGTTGGAATCG
CCTGATGCGGTATTTTCTCCTTACGCATCTGTGCGGTATTTCACACCGCATATGGT
GCACTCTCAGTACAATCTGCTCTGATGCCGCATAGTTAAGCCAGCCCCGACACCC
GCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCAT
CACCGAAACGCGCGAGACGAAAGGGCCTCGTGATACGCCTATTTTTATAGGTTA
ATGTCATGATAATAATGGTTTCTTAGACGTCAGGTGGCACTTTTCGGGGAAATGT
GCGCGGAACCCCTATTTGTTTATTTTTCTAAATACATTCAAATATGTATCCGCTCA
TGAGACAATAACCCTGATAAATGCTTCAATAATATTGAAAAAGGAAGAGTATGA
GTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGG
GTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATG
TGGCGCGGTATTATCCCGTATTGACGCCGGGCAAGAGCAACTCGGTCGCCGCAT
ACACTATTCTCAGAATGACTTGGTTGAGTACTCACCAGTCACAGAAAAGCATCTT
ACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGG
AGCTGAATGAAGCCATACCAAACGACGAGCGTGACACCACGATGCCTGTAGCAA
TGGCAACAACGTTGCGCAAACTATTAACTGGCGAACTACTTACTCTAGCTTCCCG
GCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGT
GGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCA
GCAGAGCGCAGATACCAAATACTGTCCTTCTAGTGTAGCCGTAGTTAGGCCACC
ACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATG
SEQ ID NO: 7 AAV #1262
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTGTATGACAAGCAGAAAGTAATTTGGGAGCTG
CGGGGAGGCAAGGGTAAGGGATGGGGAAGTGGACCAGAGGCATATGCGTCATT
GGCAGTGTCTAAGCACTCACGATAGGCGTGGATCACAGGGGCTCGCTCTGTAAT
TAAAAGGAAAAGGGTTTTTGTTGTGTTGTTGTTGTTGCTGTTTTTGAGACAAGGG
TCTTGCTCTGTCATCATCCAGGCTGGAGTGCAGTGGTGCAGTCTCAGCTCACTGC
AACCTCCGCCTCCTGGGTTCAAGCGATTCTCCTGCCTCAGCCTCCTGAGCAGCTA
GGACTACAGGTGTGTGCCACCATGCCTGGCTAATTTTTGTATTTTTTAGTGGAAA
TGGGGTTTTGCCATGTTGCCCAGGCTCGTCTTGAACTCCTGACCTCAAGTGATCC
ACTCGTCTCGGCCTCCCAAAGTGCTGGGATTACAGGTGTGAGCTATTGTCCCCAG
CCAAAAGGAAAAGTTTTACTGTAGTAACCCTTCCGGACTAGGGACCTCGGGCCT
CAGCCTCAGGCTACCTAGGTGCTTTAGAAAGGAGGCCACCCAGGCCCATGACTA
CTCCTTGCCACAGGGAGCCCTGCACACAGATGTGCTAAGCTCTCGCTGCCAGCCA
GAGGGAGGAGGGTCTGAGCCAGTCAGAAGGAGATGGGCCCCAGAGAGTAAGAA
AGGGGGAGGAGGACCCAAGCTGATCCAAAAGGTGGGTCTAAGCAGTCAAGTGG
AGGAGGGTTCCAATCTGATGGCGGAGGGCCCAAGCTCAGCCTAACGAGGAGGCC
AGGCCCACCAAGGGGCCCCTGGAGGACTTGTTTCCCTTGTCCCTTGTGGTTTTTT
GCATTTCCTGTTCCCTTGCTGCTCATTGCGGAAGTTCCTCTTCTTACCCTGCACCC
AGAGCCTCGCCAGAGAAGACAAGGGCAGAAAGCACCATGAGTGGGGGCCCAAT
GGGAGGAAGGCCCGGGGGCCGAGGAGCACGAACAGAGAAACAGGAGAATATG
GGCCAAACAGGATATCTGTGGTAAGCAGTTCCTGCCCCGGCTCAGGGCCAAGAA
CAGTTGGAACAGCAGAATATGGGCCAAACAGGATATCTGTGGTAAGCAGTTCCT
GCCCCGGCTCAGGGCCAAGAACAGATGGTCCCCAGATGCGGTCCCGCCCTCAGC
AGTTTCTAGAGAACCATCAGATGTTTCCAGGGTGCCCCAAGGACCTGAAATGAC
CCTGTGCCTTATTTGAACTAACCAATCAGTTCGCTTCTCGCTTCTGTTCGCGCGCT
TCTGCTCCCCGAGCTCTATATAAGCAGAGCTCGTTTAGTGAACCGTCAGATCGCC
TGGAGACGCCATCCACGCTGTTTTGACTTCCATAGAAGGATCTCGAGGCCACCAT
GGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAG
CCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACA
ACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCG
ACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGA
CGAGCTGTACAAGTAAACTAGTGTCGACTGCTTTATTTGTGAAATTTGTGATGCT
ATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAAAATC
CACCCTTCCCAGGAAGATCTCAATGTCTACTTGCCTTCCCTCTGGCTGCAGCTCTT
CCTTTGGGCCCATGACTGTCATGAGGCAGGAAGGACCAGGTCTGGCTCCAAGAC
CTTGTGGCTACCCCTGACCAGACTCCACTGACCCCTGCTTTCCTCTCCCAGACGC
TGGCCACTGCAGTTGTTCAGCTGTACCTGGCGCTGCCCCCTGGAGCTGAGCACTG
GACCAAGGAGCATTGTGGGGCTGTGTGCTTCGTGAAGGATAACCCCCAGAAGTC
CTACTTCATCCGCCTTTACGGCCTTCAGGTGACCCCCCCACCCCCGACTGGACTT
GCAAGCCAGTTCTCAACCCGCAAACCCAGATCTGTGTCCATATGTGTCCATAGCT
TCAAGACCTCAGACCTGATCAGTGAATCCCTGAGCCCCAGAACCAAAGACTCAT
CCAGATGGCAAACTCTGACTTGCCTTTCTAAGTCTGCAATGACTGGCCCCAGTCT
CCGTATCAAGATCTCTAAAGCCCCCAGTATTAGTCTGCTGCCTAAGCCTAATCTT
TTCCACAAATTCCAATAAATGAGCACTGTATTTGTACCTGAACCTCAAATCTATT
CTAAACTCAACATTTTGCATCCCAGGAATCTCTCATCAAAACTCCTGAACCCCAG
ATGTTTGCCAAGCTCCTAAGTCATAAATCTGTTCAACAAACCCCAAAGTTGAATA
TTCCATTGATCCTTGAACTCCAAATCTGTCCTTCTAAATCCACAGCACAGACCCC
AGAGTTCCCATCTAGAGCATGGCTACGTAGATAAGTAGCATGGCGGGTTAATCA
TTAACTACAAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCG
GGCGGCCTCAGTGAGCGAGCGAGCGCGCCAGCTGGCGTAATAGCGAAGAGGCC
CGCACCGATCGCCCTTCCCAACAGTTGCGCAGCCTGAATGGCGAATGGCGATTC
CGTTGCAATGGCTGGCGGTAATATTGTTCTGGATATTACCAGCAAGGCCGATAGT
TTGAGTTCTTCTACTCAGGCAAGTGATGTTATTACTAATCAAAGAAGTATTGCGA
CAACGGTTAATTTGCGTGATGGACAGACTCTTTTACTCGGTGGCCTCACTGATTA
TAAAAACACTTCTCAGGATTCTGGCGTACCGTTCCTGTCTAAAATCCCTTTAATC
GGCCTCCTGTTTAGCTCCCGCTCTGATTCTAACGAGGAAAGCACGTTATACGTGC
TCGTCAAAGCAACCATAGTACGCGCCCTGTAGCGGCGCATTAAGCGCGGCGGGT
GTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCC
CAAAAAACTTGATTAGGGTGATGGTTCACGTAGTGGGCCATCGCCCTGATAGAC
GGTTTTTCGCCCTTTGACGTTGGAGTCCACGTTCTTTAATAGTGGACTCTTGTTCC
AAACTGGAACAACACTCAACCCTATCTCGGTCTATTCTTTTGATTTATAAGGGAT
TTTGCCGATTTCGGCCTATTGGTTAAAAAATGAGCTGATTTAACAAAAATTTAAC
GCGAATTTTAACAAAATATTAACGTTTACAATTTAAATATTTGCTTATACAATCTT
CCTGTTTTTGGGGCTTTTCTGATTATCAACCGGGGTACATATGATTGACATGCTA
GTTTTACGATTACCGTTCATCGATTCTCTTGTTTGCTCCAGACTCTCAGGCAATGA
CCTGATAGCCTTTGTAGAGACCTCTCAAAAATAGCTACCCTCTCCGGCATGAATT
TATCAGCTAGAACGGTTGAATATCATATTGATGGTGATTTGACTGTCTCCGGCCT
TTCTCACCCGTTTGAATCTTTACCTACACATTACTCAGGCATTGCATTTAAAATAT
ATGAGGGTTCTAAAAATTTTTATCCTTGCGTTGAAATAAAGGCTTCTCCCGCAAA
AGTATTACAGGGTCATAATGTTTTTGGTACAACCGATTTAGCTTTATGCTCTGAG
GCTTTATTGCTTAATTTTGCTAATTCTTTGCCTTGCCTGTATGATTTATTGGATGTT
GGAATCGCCTGATGCGGTATTTTCTCCTTACGCATCTGTGCGGTATTTCACACCG
CATATGGTGCACTCTCAGTACAATCTGCTCTGATGCCGCATAGTTAAGCCAGCCC
CGACACCCGCCAACACCCGCTGACGCGCCCTGACGGGCTTGTCTGCTCCCGGCAT
CCGCTTACAGACAAGCTGTGACCGTCTCCGGGAGCTGCATGTGTCAGAGGTTTTC
ACCGTCATCACCGAAACGCGCGAGACGAAAGGGCCTCGTGATACGCCTATTTTT
ATAGGTTAATGTCATGATAATAATGGTTTCTTAGACGTCAGGTGGCACTTTTCGG
GGAAATGTGCGCGGAACCCCTATTTGTTTATTTTTCTAAATACATTCAAATATGT
ATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAG
ATCAGTTGGGTGCACGAGTGGGTTACATCGAACTGGATCTCAACAGCGGTAAGA
TCCTTGAGAGTTTTCGCCCCGAAGAACGTTTTCCAATGATGAGCACTTTTAAAGT
TCTGCTATGTGGCGCGGTATTATCCCGTATTGACGCCGGGCAAGAGCAACTCGGT
CGCCGCATACACTATTCTCAGAATGACTTGGTTGAGTACTCACCAGTCACAGAAA
AGCATCTTACGGATGGCATGACAGTAAGAGAATTATGCAGTGCTGCCATAACCA
TGAGTGATAACACTGCGGCCAACTTACTTCTGACAACGATCGGAGGACCGAAGG
AGCTAACCGCTTTTTTGCACAACATGGGGGATCATGTAACTCGCCTTGATCGTTG
GGAACCGGAGCTGAATGAAGCCATACCAAACGACGAGCGTGACACCACGATGC
CTGTAGCAATGGCAACAACGTTGCGCAAACTATTAACTGGCGAACTACTTACTCT
AGCTTCCCGGCAACAATTAATAGACTGGATGGAGGCGGATAAAGTTGCAGGACC
ACTTCTGCGCTCGGCCCTTCCGGCTGGCTGGTTTATTGCTGATAAATCTGGAGCC
GGTGAGCGTGGGTCTCGCGGTATCATTGCAGCACTGGGGCCAGATGGTAAGCCC
TCCCGTATCGTAGTTATCTACACGACGGGGAGTCAGGCAACTATGGATGAACGA
AATAGACAGATCGCTGAGATAGGTGCCTCACTGATTAAGCATTGGTAACTGTCA
GACCAAGTTTACTCATATATACTTTAGATTGATTTAAAACTTCATTTTTAATTTAA
AAGGATCTAGGTGAAGATCCTTTTTGATAATCTCATGACCAAAATCCCTTAACGT
GAGTTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTA
ACTGGCTTCAGCAGAGCGCAGATACCAAATACTGTCCTTCTAGTGTAGCCGTAGT
TAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTA
TCCGGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAA
CGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTA
ATG
SEQ ID NO: 8 AAV #1189
AAGCTTCCCGGGGGGATCTGGGCCACTCCCTCTCTGCGCGCTCGCTCGCTCACTG
AGGCCGGGCGACCAAAGGTCGCCCGACGCCCGGGCTTTGCCCGGGCGGCCTCAG
TGAGCGAGCGAGCGCGCAGAGAGGGAGTGGCCAACTCCATCACTAGGGGTTCCT
GGAGGGGTGGAGTCGTGACCTAGGCGATTTAAATTCATGTACAAAAAAGCAGGC
TTTAAAGGAACCAATTCAGTCGACTGGATCCGGTACCAAGGTCGGGCAGGAAGA
GGGCCTATTTCCCATGATTCCTTCATATTTGCATATACGATACAAGGCTGTTAGA
GAGATAATTAGAATTAATTTGACTGTAAACACAAAGATATTAGTACAAAATACG
TGACGTAGAAAGTAATAATTTCTTGGGTAGTTTGCAGTTTTAAAATTATGTTTTA
AAATGGACTATCATATGCTTACCGTAACTTGAAAGTATTTCGATTTCTTGGCTTTA
TATATCTTGTGGAAAGGACGAAACACCGGTATGTTCTGCTGAACCGCGTTTTAGA
GCTAGAAATAGCAAGTTAAAATAAGGCTAGTCCGTTATCAACTTGAAAAAGTGG
CACCGAGTCGGTGCTTTTTTTCTAGACCCAGCTTTCTTGTACAAAGTTGGCATTAA
CTAGTCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGC
AGAGAGGGACAGATCCGGGCCCGCATGCGTCGACAATTCACTGGCCGTCGTTTT
ACAACGTCGTGACTGGGAAAACCCTGGCGTTACCCAACTTAATCGCCTTGCAGC
ACATCCCCCTTTCGCCAGCTGGCGTAATAGCGAAGAGGCCCGCACCGATCGCCC
TTCCCAACAGTTGCGCAGCCTGAATGGCGAATGGCGCCTGATGCGGTATTTTCTC
CTTACGCATCTGTGCGGTATTTCACACCGCATATGGTGCACTCTCAGTACAATCT
GCTCTGATGCCGCATAGTTAAGCCAGCCCCGACACCCGCCAACACCCGCTGACG
CGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAG
ACGAAAGGGCCTCGTGATACGCCTATTTTTATAGGTTAATGTCATGATAATAATG
GTTTCTTAGACGTCAGGTGGCACTTTTCGGGGAAATGTGCGCGGAACCCCTATTT
GTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCC
GTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAG
TAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACAT
GGGGGATCATGTAACTCGCCTTGATCGTTGGGAACCGGAGCTGAATGAAGCCAT
ACCAAACGACGAGCGTGACACCACGATGCCTGTAGCAATGGCAACAACGTTGCG
CAAACTATTAACTGGCGAACTACTTACTCTAGCTTCCCGGCAACAATTAATAGAC
TGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTG
CCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAAACC
GCCTCTCCCCGCGCGTTGGCCGATTCATTAATGCAGCTGGCACGACAGGTTTCCC
GACTGGAAAGCGGGCAGTGAGCGCAACGCAATTAATGTGAGTTAGCTCACTCAT
TAGGCACCCCAGGCTTTACACTTTATGCTTCCGGCTCGTATGTTGTGTGGAATTGT
GAGCGGATAACAATTTCACACAGGAAACAGCTATGACCATGATTACGCCAAGCT
CTCGAGATCTAGA
SEQ ID NO: 9 AAV# 1215
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTTGTACAAAAAAGCAGGCTTTAAAGGAACCAA
TTCAGTCGACTGGATCCGGTACCAAGGTCGGGCAGGAAGAGGGCCTATTTCCCA
TGATTCCTTCATATTTGCATATACGATACAAGGCTGTTAGAGAGATAATTAGAAT
TAATTTGACTGTAAACACAAAGATATTAGTACAAAATACGTGACGTAGAAAGTA
ATAATTTCTTGGGTAGTTTGCAGTTTTAAAATTATGTTTTAAAATGGACTATCATA
TGCTTACCGTAACTTGAAAGTATTTCGATTTCTTGGCTTTATATATCTTGTGGAAA
GGACGAAACACCGGTATGTTCTGCTGAACCGCGTTTTAGAGCTAGAAATAGCAA
GTTAAAATAAGGCTAGTCCGTTATCAACTTGAAAAAGTGGCACCGAGTCGGTGC
TTTTTTACGCGTGTATGACAAGCAGAAAGTAATTTGGGAGCTGCGGGGAGGCAA
GGGTAAGGGATGGGGAAGTGGACCAGAGGCATATGCGTCATTGGCAGTGTCTAA
GCACTCACGATAGGCGTGGATCACAGGGGCTCGCTCTGTAATTAAAAGGAAAAG
GGTTTTTGTTGTGTTGTTGTTGTTGCTGTTTTTGAGACAAGGGTCTTGCTCTGTCA
TCATCCAGGCTGGAGTGCAGTGGTGCAGTCTCAGCTCACTGCAACCTCCGCCTCC
TGGGTTCAAGCGATTCTCCTGCCTCAGCCTCCTGAGCAGCTAGGACTACAGGTGT
GTGCCACCATGCCTGGCTAATTTTTGTATTTTTTAGTGGAAATGGGGTTTTGCCAT
GTTGCCCAGGCTCGTCTTGAACTCCTGACCTCAAGTGATCCACTCGTCTCGGCCT
CCCAAAGTGCTGGGATTACAGGTGTGAGCTATTGTCCCCAGCCAAAAGGAAAAG
TTTTACTGTAGTAACCCTTCCGGACTAGGGACCTCGGGCCTCAGCCTCAGGCTAC
CTAGGTGCTTTAGAAAGGAGGCCACCCAGGCCCATGACTACTCCTTGCCACAGG
GAGCCCTGCACACAGATGTGCTAAGCTCTCGCTGCCAGCCAGAGGGAGGAGGGT
CTGAGCCAGTCAGAAGGAGATGGGCCCCAGAGAGTAAGAAAGGGGGAGGAGGA
CCCAAGCTGATCCAAAAGGTGGGTCTAAGCAGTCAAGTGGAGGAGGGTTCCAAT
CTGATGGCGGAGGGCCCAAGCTCAGCCTAACGAGGAGGCCAGGCCCACCAAGG
GGCCCCTGGAGGACTTGTTTCCCTTGTCCCTTGTGGTTTTTTGCATTTCCTGTTCC
CTTGCTGCTCATTGCGGAAGTTCCTCTTCTTACCCTGCACCCAGAGCCTCGCCAG
AGAAGACAAGGGCAGAAAGCACCATGAGTGGGGGCCCAATGGGAGGAAGGCCC
GGGGGCCGAGGAGCACGAACAGAGAAACAGGAGAATATGGGCCAAACAGGATA
TCTGTGGTAAGCAGTTCCTGCCCCGGCTCAGGGCCAAGAACAGTTGGAACAGCA
GAATATGGGCCAAACAGGATATCTGTGGTAAGCAGTTCCTGCCCCGGCTCAGGG
CCAAGAACAGATGGTCCCCAGATGCGGTCCCGCCCTCAGCAGTTTCTAGAGAAC
CATCAGATGTTTCCAGGGTGCCCCAAGGACCTGAAATGACCCTGTGCCTTATTTG
AACTAACCAATCAGTTCGCTTCTCGCTTCTGTTCGCGCGCTTCTGCTCCCCGAGCT
CTATATAAGCAGAGCTCGTTTAGTGAACCGTCAGATCGCCTGGAGACGCCATCC
ACGCTGTTTTGACTTCCATAGAAGGATCTCGAGGCCACCATGGTGAGCAAGGGC
GAGGAGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGG
AGCGCACCATCTTCTTCAAGGACGACGGCAACTACAAGACCCGCGCCGAGGTGA
AGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCAC
AACGTCTATATCATGGCCGACAAGCAGAAGAACGGCATCAAGGTGAACTTCAAG
ATCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAGC
ACCCAGTCCGCCCTGAGCAAAGACCCCAACGAGAAGCGCGATCACATGGTCCTG
CTGGAGTTCGTGACCGCCGCCGGGATCACTCTCGGCATGGACGAGCTGTACAAG
TAAACTAGTGTCGACTGCTTTATTTGTGAAATTTGTGATGCTATTGCTTTATTTGT
AACCATTATAAGCTGCAATAAACAAGTTAACAACAACAATTGCATTCATTTTATG
TTTCAGGTTCAGGGGGAGGTGTGGGAGGTTTTTTAAACAGCGGTTCAGCAGAAC
ATACCCTCCACCCTCCTCCAGGACCACGAGAACCAGCGACTCTTTGAGATGCTTG
GACGAAAATGCTTGGTGAGCTGGGGATCTCCTGCCCCCGCCCCGTCCCCACCGTT
TCTTCCTCTTCCTCTCCTCCTTCTCTCTCTTCCCCTCCTCCCGCTCCTCCTTTCCCTC
TCCATCATCTCCTCTCCTAGAATTTCCCGTCATAATCCACCCTTCCCAGGAAGATC
TCAATGTCTACTTGCCTTCCCTCTGGCTGCAGCTCTTCCTTTGGGCCCATGACTGT
CATGAGGCAGGAAGGACCAGGTCTGGCTCCAAGACCTTGTGGCTACCCCTGACC
AGACTCCACTGACCCCTGCTTTCCTCTCCCAGACGCTGGCCACTGCAGTTGTTCA
GCTGTACCTGGCGCTGCCCCCTGGAGCTGAGCACTGGACCAAGGAGCATTGTGG
GGCTGTGTGCTTCGTGAAGGATAACCCCCAGAAGTCCTACTTCATCCGCCTTTAC
GGCCTTCAGGTGACCCCCCCACCCCCGACTGGACTTGCAAGCCAGTTCTCAACCC
GCAAACCCAGATCTGTGTCCATATGTGTCCATAGCTTCAAGACCTCAGACCTGAT
CAGTGAATCCCTGAGCCCCAGAACCAAAGACTCATCCAGATGGCAAACTCTGAC
TTGCCTTTCTAAGTCTGCAATGACTGGCCCCAGTCTCCGTATCAAGATCTCTAAA
GCCCCCAGTATTAGTCTGCTGCCTAAGCCTAATCTTTTCCACAAATTCCAATAAA
TGAGCACTGTATTTGTACCTGAACCTCAAATCTATTCTAAACTCAACATTTTGCAT
CCCAGGAATCTCTCATCAAAACTCCTGAACCCCAGATGTTTGCCAAGCTCCTAAG
TCATAAATCTGTTCAACAAACCCCAAAGTTGAATATTCCATTGATCCTTGAACTC
CAAATCTGTCCTTCTAAATCCACAGCACAGACCCCAGAGTTCCCATCTAGAGCAT
GGCTACGTAGATAAGTAGCATGGCGGGTTAATCATTAACTACAAGGAACCCCTA
GTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGC
GAGCGCGCCAGCTGGCGTAATAGCGAAGAGGCCCGCACCGATCGCCCTTCCCAA
CAGTTGCGCAGCCTGAATGGCGAATGGCGATTCCGTTGCAATGGCTGGCGGTAA
TATTGTTCTGGATATTACCAGCAAGGCCGATAGTTTGAGTTCTTCTACTCAGGCA
AGTGATGTTATTACTAATCAAAGAAGTATTGCGACAACGGTTAATTTGCGTGATG
GACAGACTCTTTTACTCGGTGGCCTCACTGATTATAAAAACACTTCTCAGGATTC
TGGCGTACCGTTCCTGTCTAAAATCCCTTTAATCGGCCTCCTGTTTAGCTCCCGCT
CTGATTCTAACGAGGAAAGCACGTTATACGTGCTCGTCAAAGCAACCATAGTAC
GCGCCCTGTAGCGGCGCATTAAGCGCGGCGGGTGTGGTGGTTACGCGCAGCGTG
ACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTA
GGGTTCCGATTTAGTGCTTTACGGCACCTCGACCCCAAAAAACTTGATTAGGGTG
ATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAAC
CCTATCTCGGTCTATTCTTTTGATTTATAAGGGATTTTGCCGATTTCGGCCTATTG
GTTAAAAAATGAGCTGATTTAACAAAAATTTAACGCGAATTTTAACAAAATATT
AACGTTTACAATTTAAATATTTGCTTATACAATCTTCCTGTTTTTGGGGCTTTTCT
GATTATCAACCGGGGTACATATGATTGACATGCTAGTTTTACGATTACCGTTCAT
CGATTCTCTTGTTTGCTCCAGACTCTCAGGCAATGACCTGATAGCCTTTGTAGAG
ACCTCTCAAAAATAGCTACCCTCTCCGGCATGAATTTATCAGCTAGAACGGTTGA
ATATCATATTGATGGTGATTTGACTGTCTCCGGCCTTTCTCACCCGTTTGAATCTT
TACCTACACATTACTCAGGCATTGCATTTAAAATATATGAGGGTTCTAAAAATTT
TTATCCTTGCGTTGAAATAAAGGCTTCTCCCGCAAAAGTATTACAGGGTCATAAT
GTTTTTGGTACAACCGATTTAGCTTTATGCTCTGAGGCTTTATTGCTTAATTTTGC
TAATTCTTTGCCTTGCCTGTATGATTTATTGGATGTTGGAATCGCCTGATGCGGTA
TTTTCTCCTTACGCATCTGTGCGGTATTTCACACCGCATATGGTGCACTCTCAGTA
CAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGT
GACCGTCTCCGGGAGCTGCATGTGTCAGAGGTTTTCACCGTCATCACCGAAACGC
GCGAGACGAAAGGGCCTCGTGATACGCCTATTTTTATAGGTTAATGTCATGATAA
TAATGGTTTCTTAGACGTCAGGTGGCACTTTTCGGGGAAATGTGCGCGGAACCCC
TATTTGTTTATTTTTCTAAATACATTCAAATATGTATCCGCTCATGAGACAATAAC
CCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCC
AGAAACGCTGGTGAAAGTAAAAGATGCTGAAGATCAGTTGGGTGCACGAGTGG
GTTACATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGTATTA
TCCCGTATTGACGCCGGGCAAGAGCAACTCGGTCGCCGCATACACTATTCTCAG
AATGACTTGGTTGAGTACTCACCAGTCACAGAAAAGCATCTTACGGATGGCATG
ACAGTAAGAGAATTATGCAGTGCTGCCATAACCATGAGTGATAACACTGCGGCC
AACTTACTTCTGACAACGATCGGAGGACCGAAGGAGCTAACCGCTTTTTTGCACA
ACATGGGGGATCATGTAACTCGCCTTGATCGTTGGGAACCGGAGCTGAATGAAG
CCATACCAAACGACGAGCGTGACACCACGATGCCTGTAGCAATGGCAACAACGT
TGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATA
GGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTT
TTTGATAATCTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAG
ATACCAAATACTGTCCTTCTAGTGTAGCCGTAGTTAGGCCACCACTTCAAGAACT
CTGTAGCACCGCCTACATACCTCGCTCTGCTAATCCTGTTACCAGTGGCTGCTGC
CAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGA
GCGAACGACCTACACCGAACTGAGATACCTACAGCGTGAGCTATGAGAAAGCGC
CACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTG
GATAACCGTATTACCGCCTTTGAGTGAGCTGATACCGCTCGCCGCAGCCGAACG
ACCGAGCGCAGCGAGTCAGTGAGCGAGGAAGCGGAAGAGCGCCCAATACGCAA
ACCGCCTCTCCCCGCGCGTTGGCCGATTCATTAATG
SEQ ID NO: 10 Sequence of Full chemically modified guide #1
5′ 2′OMe(G(ps)G(ps)U(ps)) AUG UUC UGC UGA ACC GCG UUU UAG AGC UAG AAA
UAG CAA GUU AAA AUA AGG CUA GUC CGU UAU CAA CUU GAA AAA GUG GCA
CCG AGU CGG UGC 2′OMe(U(ps)U(ps)U(ps)) U 3′
SEQ ID NO: 11 Upstream homology arm (common in #1201, #1262 and #1244)
GTATGACAAGCAGAAAGTAATTTGGGAGCTGCGGGGAGGCAAGGGTAAGGGAT
GGGGAAGTGGACCAGAGGCATATGCGTCATTGGCAGTGTCTAAGCACTCACGAT
AGGCGTGGATCACAGGGGCTCGCTCTGTAATTAAAAGGAAAAGGGTTTTTGTTG
TGTTGTTGTTGTTGCTGTTTTTGAGACAAGGGTCTTGCTCTGTCATCATCCAGGCT
GGAGTGCAGTGGTGCAGTCTCAGCTCACTGCAACCTCCGCCTCCTGGGTTCAAGC
GATTCTCCTGCCTCAGCCTCCTGAGCAGCTAGGACTACAGGTGTGTGCCACCATG
CCTGGCTAATTTTTGTATTTTTTAGTGGAAATGGGGTTTTGCCATGTTGCCCAGGC
TCGTCTTGAACTCCTGACCTCAAGTGATCCACTCGTCTCGGCCTCCCAAAGTGCT
GGGATTACAGGTGTGAGCTATTGTCCCCAGCCAAAAGGAAAAGTTTTACTGTAG
TAACCCTTCCGGACTAGGGACCTCGGGCCTCAGCCTCAGGCTACCTAGGTGCTTT
AGAAAGGAGGCCACCCAGGCCCATGACTACTCCTTGCCACAGGGAGCCCTGCAC
ACAGATGTGCTAAGCTCTCGCTGCCAGCCAGAGGGAGGAGGGTCTGAGCCAGTC
AGAAGGAGATGGGCCCCAGAGAGTAAGAAAGGGGGAGGAGGACCCAAGCTGAT
CCAAAAGGTGGGTCTAAGCAGTCAAGTGGAGGAGGGTTCCAATCTGATGGCGGA
GGGCCCAAGCTCAGCCTAACGAGGAGGCCAGGCCCACCAAGGGGCCCCTGGAG
GACTTGTTTCCCTTGTCCCTTGTGGTTTTTTGCATTTCCTGTTCCCTTGCTGCTCAT
TGCGGAAGTTCCTCTTCTTACCCTGCACCCAGAGCCTCGCCAGAGAAGACAAGG
GCAGAAAGCACCATGAGTGGGGGCCCAATGGGAGGAAGGCCCGGGGGCCGAGG
AGCAC
SEQ ID NO: 12 Downstream homology arm for #1201
CAGCGGTTCAGCAGAACATACCCTCCACCCTCCTCCAGGACCACGAGAACCAGC
GACTCTTTGAGATGCTTGGACGAAAATGCTTGGTGAGCTGGGGATCTCCTGCCCC
CGCCCCGTCCCCACCGTTTCTTCCTCTTCCTCTCCTCCTTCTCTCTCTTCCCCTCCT
CCCGCTCCTCCTTTCCCTCTCCATCATCTCCTCTCCTAGAATTTCCCGTCATAATC
CACCCTTCCCAGGAAGATCTCAATGTCTACTTGCCTTCCCTCTGGCTGCAGCTCTT
CCTTTGGGCCCATGACTGTCATGAGGCAGGAAGGACCAGGTCTGGCTCCAAGAC
CTTGTGGCTACCCCTGACCAGACTCCACTGACCCCTGCTTTCCTCTCCCAGACGC
TGGCCACTGCAGTTGTTCAGCTGTACCTGGCGCTGCCCCCTGGAGCTGAGCACTG
GACCAAGGAGCATTGTGGGGCTGTGTGCTTCGTGAAGGATAACCCCCAGAAGTC
CTACTTCATCCGCCTTTACGGCCTTCAGGTGACCCCCCCACCCCCGACTGGACTT
GCAAGCCAGTTCTCAACCCGCAAACCCAGATCTGTGTCCATATGTGTCCATAGCT
TCAAGACCTCAGACCTGATCAGTGAATCCCTGAGCCCCAGAACCAAAGACTCAT
CCAGATGGCAAACTCTGACTTGCCTTTCTAAGTCTGCAATGACTGGCCCCAGTCT
CCGTATCAAGATCTCTAAAGCCCCCAGTATTAGTCTGCTGCCTAAGCCTAATCTT
TTCCACAAATTCCAATAAATGAGCACTGTATTTGTACCTGAACCTCAAATCTATT
CTAAACTCAACATTTTGCATCCCAGGAATCTCTCATCAAAACTCCTGAACCCCAG
ATGTTTGCCAAGCTCCTAAGTCATAAATCTGTTCAACAAACCCCAAAGTTGAATA
TTCCATTGATCCTTGAACTCCAAATCTGTCCTTCTAAATCCACAGCACAGACCCC
AGAGTTCCCA
SEQ ID NO: 13 Downstream homology arm #1244
CAGCGGTTCAGCAGAACATACCCTCCACCCTCCTCCAGGACCACGAGAACCAGC
GACTCTTTGAGATGCTTGGACGAAAATGCTTGGTGAGCTGGGGATCTCCTGCCCC
CGCCCCGTCCCCACCGTTTCTTCCTCTTCCTCTCCTCCTTCTCTCTCTTCCCCTCCT
CCCGCTCCTCCTTTCCCTCTCCATCATCTCCACTCCTAGAATTTCCCGTCATAATC
CACCCTTCCCAGGAAGATCTCAATGTCTTCTTGCCTTCCCTCTGGCTGCAGCTCTT
CCTTTGGGCCCATGACTGTCATGAGGCAGGAAGGACCAGGTCTGGCTCCAAGAC
CTTGTGGCTACCCCTGACCAGACTCCACTGACCCCTGCTTTCCTCTCCCAGACGC
TGGCCACTGCAGTTGTTCAGCTGTACCTGGCGCTGCCCCCTGGAGCTGAGCACTG
GACCAAGGAGCATTGTGGGGCTGTGTGCTTCGTGAAGGATAACCCCCAGAAGTC
CTACTTCATCCGCCTTTACGGCCTTCAGGTGACCCCCCCACCCCCGACTGGACTT
GCAAGCCAGTTCTCAACCCGCAAACCCAGATCTGTGTCCATATGTGTCCATAGCT
TCAAGACCTCAGACCTGATCAGTGAATCCCTGAGCCCCAGAACCAAAGACTCAT
CCAGATGGCAAACTCTGACTTGCCTTTCTAAGTCTGCAATGACTGGCCCCAGTCT
CCGTATCAAGATCTCTAAAGCCCCCAGTATTAGTCTGCTGCCTAAGCCTAATCTT
TTCCACAAATTCCAATAAATGAGCACTGTATTTGTACCTGAACCTCAAATCTATT
CTAAACTCAACATTTTGCATCCCAGGAATCTCTCATCAAAACTCCTGAACCCCAG
ATGTTTGCCAAGCTCCTAAGTCATAAATCTGTTCAACAAACCCCAAAGTTGAATA
TTCCATTGATCCTTGAACTCCAAATCTGTCCTTCTAAATCCACAGCACAGACCCC
AGAGTTCCCA
SEQ ID NO: 14 Downstream homology arm #1262
AATCCACCCTTCCCAGGAAGATCTCAATGTCTACTTGCCTTCCCTCTGGCTGCAG
CTCTTCCTTTGGGCCCATGACTGTCATGAGGCAGGAAGGACCAGGTCTGGCTCCA
AGACCTTGTGGCTACCCCTGACCAGACTCCACTGACCCCTGCTTTCCTCTCCCAG
ACGCTGGCCACTGCAGTTGTTCAGCTGTACCTGGCGCTGCCCCCTGGAGCTGAGC
ACTGGACCAAGGAGCATTGTGGGGCTGTGTGCTTCGTGAAGGATAACCCCCAGA
AGTCCTACTTCATCCGCCTTTACGGCCTTCAGGTGACCCCCCCACCCCCGACTGG
ACTTGCAAGCCAGTTCTCAACCCGCAAACCCAGATCTGTGTCCATATGTGTCCAT
AGCTTCAAGACCTCAGACCTGATCAGTGAATCCCTGAGCCCCAGAACCAAAGAC
TCATCCAGATGGCAAACTCTGACTTGCCTTTCTAAGTCTGCAATGACTGGCCCCA
GTCTCCGTATCAAGATCTCTAAAGCCCCCAGTATTAGTCTGCTGCCTAAGCCTAA
TCTTTTCCACAAATTCCAATAAATGAGCACTGTATTTGTACCTGAACCTCAAATC
TATTCTAAACTCAACATTTTGCATCCCAGGAATCTCTCATCAAAACTCCTGAACC
CCAGATGTTTGCCAAGCTCCTAAGTCATAAATCTGTTCAACAAACCCCAAAGTTG
AATATTCCATTGATCCTTGAACTCCAAATCTGTCCTTCTAAATCCACAGCACAGA
CCCCAGAGTTCCCA
SEQ ID NO: 15 WAS TALEN binding site on the genome (forward-TALEN)
CTCCTAGAATTTCCCGT
SEQ ID NO: 16 WAS TALEN binding site on the genome (reverse-TALEN)
AGGAAGATCTCAATGTCT
SEQ ID NO: 17 TALEN cleavage site (spacer sequence)
CATAATCCACCCTTCCC
SEQ ID NO: 18 WAS exon 2
ACGCTGGCCACTGCAGTTGTTCAGCTGTACCTGGCGCTGCCCCCTGGAGCTGAGC
ACTGGACCAAGGAGCATTGTGGGGCTGTGTGCTTCGTGAAGGATAACCCCCAGA
AGTCCTACTTCATCCGCCTTTACGGCCTTCAG
SEQ ID NO: 19 WAS exon 1 sequence
TCCTCTTCTTACCCTGCACCCAGAGCCTCGCCAGAGAAGACAAGGGCAGAAAGC
ACCATGAGTGGGGGCCCAATGGGAGGAAGGCCCGGGGGCCGAGGAGCACCAGC
GGTTCAGCAGAACATACCCTCCACCCTCCTCCAGGACCACGAGAACCAGCGACT
CTTTGAGATGCTTGGACGAAAATGCTTG
SEQ ID NO: 20 pWNY.2xNLS.Cas9.mCherry
TCGCGCGTTTCGGTGATGACGGTGAAAACCTCTGACACATGCAGCTCCCGGAGA
CGGTCACAGCTTGTCTGTAAGCGGATGCCGGGAGCAGACAAGCCCGTCAGGGCG
CGTCAGCGGGTGTTGGCGGGTGTCGGGGCTGGCTTAACTATGCGGCATCAGAGC
AGATTGTACTGAGAGTGCACCATACACTTAGTGTAATACGACTCACTATAGGGA
GAGCGGCCGCTTTTTCAGCAAGATTAAGCCGCCACCATGGCGCCGCGGCCTCCT
AAGAAGAAGCGGAAAGTCGAATTCTACGTAATGGACAAGAAGTACTCCATTGGG
CTCGATATCGGCACAAACAGCGTCGGCTGGGCCGTCATTACGGACGAGTACAAG
GTGCCGAGCAAAAAATTCAAAGTTCTGGGCAATACCGATCGCCACAGCATAAAG
AAGAACCTCATTGGCGCCCTCCTGTTCGACTCCGGGGAGACGGCCGAAGCCACG
CGGCTCAAAAGAACAGCACGGCGCAGATATACCCGCAGAAAGAATCGGATCTG
CTACCTGCAGGAGATCTTTAGTAATGAGATGGCTAAGGTGGATGACTCTTTCTTC
CATAGGCTGGAGGAGTCCTTTTTGGTGGAGGAGGATAAAAAGCACGAGCGCCAC
CCAATCTTTGGCAATATCGTGGACGAGGTGGCGTACCATGAAAAGTACCCAACC
ATATATCATCTGAGGAAGAAGCTTGTAGACAGTACTGATAAGGCTGACTTGCGG
TTGATCTATCTCGCGCTGGCGCATATGATCAAATTTCGGGGACACTTCCTCATCG
AGGGGGACCTGAACCCAGACAACAGCGATGTCGACAAACTCTTTATCCAACTGG
TTCAGACTTACAATCAGCTTTTCGAAGAGAACCCGATCAACGCATCCGGAGTTGA
CGCCAAAGCAATCCTGAGCGCTAGGCTGTCCAAATCCCGGCGGCTCGAAAACCT
CATCGCACAGCTCCCTGGGGAGAAGAAGAACGGCCTGTTTGGTAATCTTATCGC
CCTGTCACTCGGGCTGACCCCCAACTTTAAATCTAACTTCGACCTGGCCGAAGAT
GCCAAGCTTCAACTGAGCAAAGACACCTACGATGATGATCTCGACAATCTGCTG
GCCCAGATCGGCGACCAGTACGCAGACCTTTTTTTGGCGGCAAAGAACCTGTCA
GACGCCATTCTGCTGAGTGATATTCTGCGAGTGAACACGGAGATCACCAAAGCT
CCGCTGAGCGCTAGTATGATCAAGCGCTATGATGAGCACCACCAAGACTTGACT
TTGCTGAAGGCCCTTGTCAGACAGCAACTGCCTGAGAAGTACAAGGAAATTTTCT
TCGATCAGTCTAAAAATGGCTACGCCGGATACATTGACGGCGGAGCAAGCCAGG
AGGAATTTTACAAATTTATTAAGCCCATCTTGGAAAAAATGGACGGCACCGAGG
AGCTGCTGGTAAAGCTTAACAGAGAAGATCTGTTGCGCAAACAGCGCACTTTCG
ACAATGGAAGCATCCCCCACCAGATTCACCTGGGCGAACTGCACGCTATCCTCA
GGCGGCAAGAGGATTTCTACCCCTTTTTGAAAGATAACAGGGAAAAGATTGAGA
AAATCCTCACATTTCGGATACCCTACTATGTAGGCCCCCTCGCCCGGGGAAATTC
CAGATTCGCGTGGATGACTCGCAAATCAGAAGAGACAATCACTCCCTGGAACTT
CGAGGAAGTCGTGGATAAGGGGGCCTCTGCCCAGTCCTTCATCGAAAGGATGAC
TAACTTTGATAAAAATCTGCCTAACGAAAAGGTGCTTCCTAAACACTCTCTGCTG
TACGAGTACTTCACAGTTTATAACGAGCTCACCAAGGTCAAATACGTCACAGAA
GGGATGAGAAAGCCAGCATTCCTGTCTGGAGAGCAGAAGAAAGCTATCGTGGAC
CTCCTCTTCAAGACGAACCGGAAAGTTACCGTGAAACAGCTCAAAGAAGACTAT
TTCAAAAAGATTGAATGTTTCGACTCTGTTGAAATCAGCGGAGTGGAGGATCGCT
TCAACGCATCCCTGGGAACGTATCACGATCTCCTGAAAATCATTAAAGACAAGG
ACTTCCTGGACAATGAGGAGAACGAGGACATTCTTGAGGACATTGTCCTCACCCT
TACGTTGTTTGAAGATAGGGAGATGATTGAAGAACGCTTGAAAACTTACGCTCA
TCTCTTCGACGACAAAGTCATGAAACAGCTCAAGAGGCGCCGATATACAGGATG
GGGGCGGCTGTCAAGAAAACTGATCAATGGGATCCGAGACAAGCAGAGTGGAA
AGACAATCCTGGATTTTCTTAAGTCCGATGGATTTGCCAACCGGAACTTCATGCA
GTTGATCCATGATGACTCTCTCACCTTTAAGGAGGACATCCAGAAAGCACAAGTT
TCTGGCCAGGGGGACAGTCTTCACGAGCACATCGCTAATCTTGCAGGTAGCCCA
GCTATCAAAAAGGGAATACTGCAGACCGTTAAGGTCGTGGATGAACTCGTCAAA
GTAATGGGAAGGCATAAGCCCGAGAATATCGTTATCGAGATGGCCCGAGAGAAC
CAAACTACCCAGAAGGGACAGAAGAACAGTAGGGAAAGGATGAAGAGGATTGA
AGAGGGTATAAAAGAACTGGGGTCCCAAATCCTTAAGGAACACCCAGTTGAAAA
CACCCAGCTTCAGAATGAGAAGCTCTACCTGTACTACCTGCAGAACGGCAGGGA
CATGTACGTGGATCAGGAACTGGACATCAATCGGCTCTCCGACTACGACGTGGA
TCATATCGTGCCCCAGTCTTTTCTCAAAGATGATTCTATTGATAATAAAGTGTTG
ACAAGATCCGATAAAAATAGAGGGAAGAGTGATAACGTCCCCTCAGAAGAAGTT
GTCAAGAAAATGAAAAATTATTGGCGGCAGCTGCTGAACGCCAAACTGATCACA
CAACGGAAGTTCGATAATCTGACTAAGGCTGAACGAGGTGGCCTGTCTGAGTTG
GATAAAGCCGGCTTCATCAAAAGGCAGCTTGTTGAGACACGCCAGATCACCAAG
CACGTGGCCCAAATTCTCGATTCACGCATGAACACCAAGTACGATGAAAATGAC
AAACTGATTCGAGAGGTGAAAGTTATTACTCTGAAGTCTAAGCTGGTGTCCGATT
TCAGAAAGGACTTTCAGTTTTATAAGGTGAGAGAGATCAACAATTACCACCATG
CGCATGATGCCTACCTGAATGCAGTGGTAGGCACTGCACTTATCAAAAAATATCC
CAAGCTTGAATCTGAATTTGTTTACGGAGACTATAAAGTGTACGATGTTAGGAAA
ATGATCGCAAAGTCTGAGCAGGAAATAGGCAAGGCCACCGCTAAGTACTTCTTT
TACAGCAATATTATGAATTTTTTCAAGACCGAGATTACACTGGCCAATGGAGAG
ATTCGGAAGCGACCACTTATCGAAACAAACGGAGAAACAGGAGAAATCGTGTG
GGACAAGGGTAGGGATTTCGCGACAGTCCGGAAGGTCCTGTCCATGCCGCAGGT
GAACATCGTTAAAAAGACCGAAGTACAGACCGGAGGCTTCTCCAAGGAAAGTAT
CCTCCCGAAAAGGAACAGCGACAAGCTGATCGCACGCAAAAAAGATTGGGACC
CCAAGAAATACGGCGGATTCGATTCTCCTACAGTCGCTTACAGTGTACTGGTTGT
GGCCAAAGTGGAGAAAGGGAAGTCTAAAAAACTCAAAAGCGTCAAGGAACTGC
TGGGCATCACAATCATGGAGCGATCAAGCTTCGAAAAAAACCCCATCGACTTTC
TCGAGGCGAAAGGATATAAAGAGGTCAAAAAAGACCTCATCATTAAGCTTCCCA
AGTACTCTCTCTTTGAGCTTGAAAACGGCCGGAAACGAATGCTCGCTAGTGCGG
GCGAGCTGCAGAAAGGTAACGAGCTGGCACTGCCCTCTAAATACGTTAATTTCTT
GTATCTGGCCAGCCACTATGAAAAGCTCAAAGGCTCTCCCGAAGATAATGAGCA
GAAGCAGCTGTTCGTGGAACAACACAAACACTACCTTGATGAGATCATCGAGCA
AATAAGCGAATTCTCCAAAAGAGTGATCCTCGCCGACGCTAACCTCGATAAGGT
GCTTTCTGCTTACAATAAGCACAGGGATAAGCCCATCAGGGAGCAGGCAGAAAA
CATTATCCACTTGTTTACTCTGACCAACTTGGGCGCGCCTGCAGCCTTCAAGTAC
TTCGACACCACCATAGACAGAAAGCGGTACACCTCTACAAAGGAGGTCCTGGAC
GCCACACTGATTCATCAGTCAATTACGGGGCTCTATGAAACAAGAATCGACCTCT
CTCAGCTCGGTGGAGACAGCAGGGCTGACCCCAAGAAGAAGAGGAAGGTGGCT
AGCGGAAGCGGAGGTGAGGGCAGAGGAAGTCTTCTAACATGCGGTGACGTGGA
GGAGAATCCGGGCCCTGTGAGCAAGGGCGAGGAGGATAACATGGCCATCATCA
AGGAGTTCATGCGCTTCAAGGTGCACATGGAGGGCTCCGTGAACGGCCACGAGT
TCGAGATCGAGGGCGAGGGCGAGGGCCGCCCCTACGAGGGCACCCAGACCGCC
AAGCTGAAGGTGACCAAGGGTGGCCCCCTGCCCTTCGCCTGGGACATCCTGTCC
CCTCAGTTCATGTACGGCTCCAAGGCCTACGTGAAGCACCCCGCCGACATCCCCG
ACTACTTGAAGCTGTCCTTCCCCGAGGGCTTCAAGTGGGAGCGCGTGATGAACTT
CGAGGACGGCGGCGTGGTGACCGTGACCCAGGACTCCTCTCTGCAGGACGGCGA
GTTCATCTACAAGGTGAAGCTGCGCGGCACCAACTTCCCCTCCGACGGCCCCGTA
ATGCAGAAGAAGACCATGGGCTGGGAGGCCTCCTCCGAGCGGATGTACCCCGAG
GACGGCGCCCTGAAGGGCGAGATCAAGCAGAGGCTGAAGCTGAAGGACGGCGG
CCACTACGACGCTGAGGTCAAGACCACCTACAAGGCCAAGAAGCCCGTGCAGCT
GCCCGGCGCCTACAACGTCAACATCAAGTTGGACATCACCTCCCACAACGAGGA
CTACACCATCGTGGAACAGTACGAACGCGCCGAGGGCCGCCACTCCACCGGCGG
CATGGACGAGCTGTACAAGTGAGGTACCCGTACGAGCTTGGCGTAATCATGGTC
ATAGCTGTTTCCTGTGTGAAATTGTTATCCGCTCACAATTCCACACAACATACGA
GCCGGAAGCATAAAGTGTAAAGCCTGGGGTGCCTAATGAGTGAGCTAACTCACA
TTAATTGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTT
GAGTCCAACCCGGTAAGACACGACTTATCGCCACTGGCAGCAGCCACTGGTAAC
AGGATTAGCAGAGCGAGGTATGTAGGCGGTGCTACAGAGTTCTTGAAGTGGTGG
CCTAACTACGGCTACACTAGAAGAACAGTATTTGGTATCTGCGCTCTGCTGAAGC
CAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGA
AAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTG
TCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCT
CACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGA
AGCTAGAGTAAGTAGTTCGCCAGTTAATAGTTTGCGCAACGTTGTTGCCATTGCT
ACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACT
CATGGTTATGGCAGCACTGCATAATTCTCTTACTGTCATGCCATCCGTAAGATGC
TTTTCTGTGACTGGTGAGTACTCAACCAAGTCATTCTGAGAATAGTGTATGCGGC
GACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTG
ATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCG
GATACATATTTGAATGTATTTAGAAAAATAAACAAATAGGGGTTCCGCGCACAT
TTCCCCGAAAAGTGCCACCTGACGTCTAAGAAACCATTATTATCATGACATTAAC
CTATAAAAATAGGCGTATCACGAGGCCCTTTCGTC
SEQ ID NO: 21 1190 scAAV.U6.guideRNA2
AAGCTTCCCGGGGGGATCTGGGCCACTCCCTCTCTGCGCGCTCGCTCGCTCACTG
AGGCCGGGCGACCAAAGGTCGCCCGACGCCCGGGCTTTGCCCGGGCGGCCTCAG
TGAGCGAGCGAGCGCGCAGAGAGGGAGTGGCCAACTCCATCACTAGGGGTTCCT
GGAGGGGTGGAGTCGTGACCTAGGCGATTTAAATTCATGTACAAAAAAGCAGGC
TTTAAAGGAACCAATTCAGTCGACTGGATCCGGTACCAAGGTCGGGCAGGAAGA
GGGCCTATTTCCCATGATTCCTTCATATTTGCATATACGATACAAGGCTGTTAGA
GAGATAATTAGAATTAATTTGACTGTAAACACAAAGATATTAGTACAAAATACG
TGACGTAGAAAGTAATAATTTCTTGGGTAGTTTGCAGTTTTAAAATTATGTTTTA
AAATGGACTATCATATGCTTACCGTAACTTGAAAGTATTTCGATTTCTTGGCTTTA
TATATCTTGTGGAAAGGACGAAACACCGCAAAGAGTCGCTGGTTCTCGGTTTTAG
AGCTAGAAATAGCAAGTTAAAATAAGGCTAGTCCGTTATCAACTTGAAAAAGTG
GCACCGAGTCGGTGCTTTTTTTCTAGACCCAGCTTTCTTGTACAAAGTTGGCATTA
ACTAGTCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCG
CAGAGAGGGACAGATCCGGGCCCGCATGCGTCGACAATTCACTGGCCGTCGTTT
TACAACGTCGTGACTGGGAAAACCCTGGCGTTACCCAACTTAATCGCCTTGCAGC
ACATCCCCCTTTCGCCAGCTGGCGTAATAGCGAAGAGGCCCGCACCGATCGCCC
TTCCCAACAGTTGCGCAGCCTGAATGGCGAATGGCGCCTGATGCGGTATTTTCTC
CTTACGCATCTGTGCGGTATTTCACACCGCATATGGTGCACTCTCAGTACAATCT
GCTCTGATGCCGCATAGTTAAGCCAGCCCCGACACCCGCCAACACCCGCTGACG
CGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAG
ACGAAAGGGCCTCGTGATACGCCTATTTTTATAGGTTAATGTCATGATAATAATG
GTTTCTTAGACGTCAGGTGGCACTTTTCGGGGAAATGTGCGCGGAACCCCTATTT
GTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCC
GTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAG
TAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACAT
GGGGGATCATGTAACTCGCCTTGATCGTTGGGAACCGGAGCTGAATGAAGCCAT
ACCAAACGACGAGCGTGACACCACGATGCCTGTAGCAATGGCAACAACGTTGCG
CAAACTATTAACTGGCGAACTACTTACTCTAGCTTCCCGGCAACAATTAATAGAC
TGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTG
CCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAAACC
GCCTCTCCCCGCGCGTTGGCCGATTCATTAATGCAGCTGGCACGACAGGTTTCCC
GACTGGAAAGCGGGCAGTGAGCGCAACGCAATTAATGTGAGTTAGCTCACTCAT
TAGGCACCCCAGGCTTTACACTTTATGCTTCCGGCTCGTATGTTGTGTGGAATTGT
GAGCGGATAACAATTTCACACAGGAAACAGCTATGACCATGATTACGCCAAGCT
CTCGAGATCTAGA
SEQ ID NO: 22 #1191 scAAV.U6.guideRNA3
AAGCTTCCCGGGGGGATCTGGGCCACTCCCTCTCTGCGCGCTCGCTCGCTCACTG
AGGCCGGGCGACCAAAGGTCGCCCGACGCCCGGGCTTTGCCCGGGCGGCCTCAG
TGAGCGAGCGAGCGCGCAGAGAGGGAGTGGCCAACTCCATCACTAGGGGTTCCT
GGAGGGGTGGAGTCGTGACCTAGGCGATTTAAATTCATGTACAAAAAAGCAGGC
TTTAAAGGAACCAATTCAGTCGACTGGATCCGGTACCAAGGTCGGGCAGGAAGA
GGGCCTATTTCCCATGATTCCTTCATATTTGCATATACGATACAAGGCTGTTAGA
GAGATAATTAGAATTAATTTGACTGTAAACACAAAGATATTAGTACAAAATACG
TGACGTAGAAAGTAATAATTTCTTGGGTAGTTTGCAGTTTTAAAATTATGTTTTA
AAATGGACTATCATATGCTTACCGTAACTTGAAAGTATTTCGATTTCTTGGCTTTA
TATATCTTGTGGAAAGGACGAAACACCGCAAGCATCTCAAAGAGTCGCGTTTTA
GAGCTAGAAATAGCAAGTTAAAATAAGGCTAGTCCGTTATCAACTTGAAAAAGT
GGCACCGAGTCGGTGCTTTTTTTCTAGACCCAGCTTTCTTGTACAAAGTTGGCATT
AACTAGTCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGC
GCAGAGAGGGACAGATCCGGGCCCGCATGCGTCGACAATTCACTGGCCGTCGTT
TTACAACGTCGTGACTGGGAAAACCCTGGCGTTACCCAACTTAATCGCCTTGCAG
CACATCCCCCTTTCGCCAGCTGGCGTAATAGCGAAGAGGCCCGCACCGATCGCC
CTTCCCAACAGTTGCGCAGCCTGAATGGCGAATGGCGCCTGATGCGGTATTTTCT
CCTTACGCATCTGTGCGGTATTTCACACCGCATATGGTGCACTCTCAGTACAATC
TGCTCTGATGCCGCATAGTTAAGCCAGCCCCGACACCCGCCAACACCCGCTGAC
GCGCCCTGACGGGCTTGTCTGCTCCCGGCATCCGCTTACAGACAAGCTGTGACCG
TCTCCGGGAGCTGCATGTGTCAGAGGTTTTCACCGTCATCACCGAAACGCGCGA
GACGAAAGGGCCTCGTGATACGCCTATTTTTATAGGTTAATGTCATGATAATAAT
GGTTTCTTAGACGTCAGGTGGCACTTTTCGGGGAAATGTGCGCGGAACCCCTATT
TGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCC
GTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAG
TAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACAT
GGGGGATCATGTAACTCGCCTTGATCGTTGGGAACCGGAGCTGAATGAAGCCAT
ACCAAACGACGAGCGTGACACCACGATGCCTGTAGCAATGGCAACAACGTTGCG
CAAACTATTAACTGGCGAACTACTTACTCTAGCTTCCCGGCAACAATTAATAGAC
TGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTG
CCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAAACC
GCCTCTCCCCGCGCGTTGGCCGATTCATTAATGCAGCTGGCACGACAGGTTTCCC
GACTGGAAAGCGGGCAGTGAGCGCAACGCAATTAATGTGAGTTAGCTCACTCAT
TAGGCACCCCAGGCTTTACACTTTATGCTTCCGGCTCGTATGTTGTGTGGAATTGT
GAGCGGATAACAATTTCACACAGGAAACAGCTATGACCATGATTACGCCAAGCT
CTCGAGATCTAGA
SEQ ID NO: 23 #1192 scAAV.U6.guideRNA4
AAGCTTCCCGGGGGGATCTGGGCCACTCCCTCTCTGCGCGCTCGCTCGCTCACTG
AGGCCGGGCGACCAAAGGTCGCCCGACGCCCGGGCTTTGCCCGGGCGGCCTCAG
TGAGCGAGCGAGCGCGCAGAGAGGGAGTGGCCAACTCCATCACTAGGGGTTCCT
GGAGGGGTGGAGTCGTGACCTAGGCGATTTAAATTCATGTACAAAAAAGCAGGC
TTTAAAGGAACCAATTCAGTCGACTGGATCCGGTACCAAGGTCGGGCAGGAAGA
GGGCCTATTTCCCATGATTCCTTCATATTTGCATATACGATACAAGGCTGTTAGA
GAGATAATTAGAATTAATTTGACTGTAAACACAAAGATATTAGTACAAAATACG
TGACGTAGAAAGTAATAATTTCTTGGGTAGTTTGCAGTTTTAAAATTATGTTTTA
AAATGGACTATCATATGCTTACCGTAACTTGAAAGTATTTCGATTTCTTGGCTTTA
TATATCTTGTGGAAAGGACGAAACACCGACCATGAGTGGGGGCCCAATGTTTTA
GAGCTAGAAATAGCAAGTTAAAATAAGGCTAGTCCGTTATCAACTTGAAAAAGT
GGCACCGAGTCGGTGCTTTTTTTCTAGACCCAGCTTTCTTGTACAAAGTTGGCATT
AACTAGTCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGC
GCAGAGAGGGACAGATCCGGGCCCGCATGCGTCGACAATTCACTGGCCGTCGTT
TTACAACGTCGTGACTGGGAAAACCCTGGCGTTACCCAACTTAATCGCCTTGCAG
CACATCCCCCTTTCGCCAGCTGGCGTAATAGCGAAGAGGCCCGCACCGATCGCC
CTTCCCAACAGTTGCGCAGCCTGAATGGCGAATGGCGCCTGATGCGGTATTTTCT
CCTTACGCATCTGTGCGGTATTTCACACCGCATATGGTGCACTCTCAGTACAATC
TGCTCTGATGCCGCATAGTTAAGCCAGCCCCGACACCCGCCAACACCCGCTGAC
GCGCCCTGACGGGCTTGTCTGCTCCCGGCATCCGCTTACAGACAAGCTGTGACCG
TCTCCGGGAGCTGCATGTGTCAGAGGTTTTCACCGTCATCACCGAAACGCGCGA
GACGAAAGGGCCTCGTGATACGCCTATTTTTATAGGTTAATGTCATGATAATAAT
GGTTTCTTAGACGTCAGGTGGCACTTTTCGGGGAAATGTGCGCGGAACCCCTATT
TGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCC
GTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAG
TAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACAT
GGGGGATCATGTAACTCGCCTTGATCGTTGGGAACCGGAGCTGAATGAAGCCAT
ACCAAACGACGAGCGTGACACCACGATGCCTGTAGCAATGGCAACAACGTTGCG
CAAACTATTAACTGGCGAACTACTTACTCTAGCTTCCCGGCAACAATTAATAGAC
TGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTG
CCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAAACC
GCCTCTCCCCGCGCGTTGGCCGATTCATTAATGCAGCTGGCACGACAGGTTTCCC
GACTGGAAAGCGGGCAGTGAGCGCAACGCAATTAATGTGAGTTAGCTCACTCAT
TAGGCACCCCAGGCTTTACACTTTATGCTTCCGGCTCGTATGTTGTGTGGAATTGT
GAGCGGATAACAATTTCACACAGGAAACAGCTATGACCATGATTACGCCAAGCT
CTCGAGATCTAGA
SEQ ID NO: 24 WAS TALEN #2 forward
ATGGCGCCGCGGCCTCCTAAGAAGAAGCGGAAAGTCGAATTCGTGGATCTGCGA
ACACTGGGCTATAGCCAGCAGCAGCAGGAGAAGATCAAACCCAAGGTGAGGTC
CACAGTCGCACAGCACCATGAAGCCCTGGTGGGCCACGGGTTCACTCACGCTCA
TATTGTCGCACTGTCTCAGCATCCAGCCGCTCTGGGAACCGTGGCAGTCACATAC
CAGCACATCATTACTGCCCTGCCCGAGGCTACCCATGAAGACATCGTGGGAGTC
GGCAAACAGTGGAGCGGCGCACGGGCCCTGGAGGCTCTGCTGACCGACGCAGG
GGAACTGAGAGGACCCCCTCTGCAGCTGGATACAGGGCAGCTGGTGAAGATTGC
TAAGAGGGGAGGGGTGACAGCAATGGAAGCCGTCCACGCAAGCAGGAACGCAC
TGACAGGGGCCCCCCTGAACCTGACCCCGGACCAAGTGGTGGCTATCGCCAGCC
ACGATGGCGGCAAGCAAGCGCTCGAAACGGTGCAGCGGCTGTTGCCGGTGCTGT
GCCAGGACCATGGCCTGACCCCGGACCAAGTGGTGGCTATCGCCAGCAACGGTG
GCGGCAAGCAAGCGCTCGAAACGGTGCAGCGGCTGTTGCCGGTGCTGTGCCAGG
ACCATGGCCTGACCCCGGACCAAGTGGTGGCTATCGCCAGCAACAATGGCGGCA
AGCAAGCGCTCGAAACGGTGCAGCGGCTGTTGCCGGTGCTGTGCCAGGACCATG
GCCTGACCCCGGACCAAGTGGTGGCTATCGCCAGCAACAATGGCGGCAAGCAAG
CGCTCGAAACGGTGCAGCGGCTGTTGCCGGTGCTGTGCCAGGACCATGGCCTGA
CTCCGGACCAAGTGGTGGCTATCGCCAGCCACGATGGCGGCAAGCAAGCGCTCG
AAACGGTGCAGCGGCTGTTGCCGGTGCTGTGCCAGGACCATGGCCTGACCCCGG
ACCAAGTGGTGGCTATCGCCAGCAACGGTGGCGGCAAGCAAGCGCTCGAAACG
GTGCAGCGGCTGTTGCCGGTGCTGTGCCAGGACCATGGCCTGACTCCGGACCAA
GTGGTGGCTATCGCCAGCAACAATGGCGGCAAGCAAGCGCTCGAAACGGTGCAG
CGGCTGTTGCCGGTGCTGTGCCAGGACCATGGCCTGACCCCGGACCAAGTGGTG
GCTATCGCCAGCCACGATGGCGGCAAGCAAGCGCTCGAAACGGTGCAGCGGCTG
TTGCCGGTGCTGTGCCAGGACCATGGCCTGACCCCGGACCAAGTGGTGGCTATC
GCCAGCAACATTGGCGGCAAGCAAGCGCTCGAAACGGTGCAGCGGCTGTTGCCG
GTGCTGTGCCAGGACCATGGCCTGACTCCGGACCAAGTGGTGGCTATCGCCAGC
AACAATGGCGGCAAGCAAGCGCTCGAAACGGTGCAGCGGCTGTTGCCGGTGCTG
TGCCAGGACCATGGCCTGACCCCGGACCAAGTGGTGGCTATCGCCAGCCACGAT
GGCGGCAAGCAAGCGCTCGAAACGGTGCAGCGGCTGTTGCCGGTGCTGTGCCAG
GACCATGGCCTGACCCCGGACCAAGTGGTGGCTATCGCCAGCAACGGTGGCGGC
AAGCAAGCGCTCGAAACGGTGCAGCGGCTGTTGCCGGTGCTGTGCCAGGACCAT
GGCCTGACCCCGGACCAAGTGGTGGCTATCGCCAGCCACGATGGCGGCAAGCAA
GCGCTCGAAACGGTGCAGCGGCTGTTGCCGGTGCTGTGCCAGGACCATGGCCTG
ACCCCGGACCAAGTGGTGGCTATCGCCAGCAACGGTGGCGGCAAGCAAGCGCTC
GAAACGGTGCAGCGGCTGTTGCCGGTGCTGTGCCAGGACCATGGCCTGACCCCG
GACCAAGTGGTGGCTATCGCCAGCAACGGTGGCGGCAAGCAAGCGCTCGAAAC
GGTGCAGCGGCTGTTGCCGGTGCTGTGCCAGGACCATGGCCTGACTCCGGACCA
AGTGGTGGCTATCGCCAGCCACGATGGCGGCAAGCAAGCGCTCGAAACGGTGCA
GCGGCTGTTGCCGGTGCTGTGCCAGGACCATGGCCTGACCCCGGACCAAGTGGT
GGCTATCGCCAGCCACGATGGCGGCAAGCAAGCGCTCGAAACGGTGCAGCGGCT
GTTGCCGGTGCTGTGCCAGGACCATGGCCTGACCCCGGACCAAGTGGTGGCTAT
CGCCAGCAACGGTGGCGGCAAGCAAGCGCTCGAAAGCATTGTGGCCCAGCTGAG
CCGGCCTGATCCGGCGTTGGCCGCGTTGACCAACGACCACCTGGTCGCTCTGGCT
TGCCTGGGAGGACGCCCTGCTATGGACGCTGTGAAGAAAGGACTGCCCCACGCA
CCCGAACTGATTAGACGGGTGAACCGGAGAATCGGCGAGAGAACATCCCATAG
GGTGGCAATCTCTAGAACTCAGCTGGTCAAGAGTGAACTGGAGGAAAAGAAATC
AGAGCTGCGCCACAAGCTGAAATACGTGCCTCATGAGTATATCGAACTGATCGA
GATTGCTCGCAATTCAACCCAGGACCGGATCCTGGAAATGAAAGTGATGGAGTT
CTTTATGAAAGTCTACGGATATCGGGGGAAACACCTGGGAGGGAGCAGAAAGCC
AGATGGGGCCATCTACACAGTGGGATCCCCCATCGACTATGGCGTGATTGTCGAT
ACTAAAGCCTACAGCGGAGGCTATAACCTGCCTATCGGCCAGGCTGACGAGATG
CAGAGATACGTGGAGGAAAACCAGACCCGCAATAAGCATATTAACCCCAATGA
ATGGTGGAAAGTGTATCCTAGCTCCGTCACAGAGTTCAAGTTTCTGTTCGTGAGC
GGACACTTTAAGGGCAACTACAAAGCACAGCTGACTAGGCTGAATCATATCACC
AACTGCAATGGAGCCGTGCTGTCTGTCGAGGAACTGCTGATCGGGGGAGAGATG
ATTAAGGCTGGCACACTGACTCTGGAGGAAGTGAGGCGCAAGTTCAACAATGGG
GAAATCAACTTCTAA
SEQ ID NO: 25 WAS TALEN #2 Reverse
ATGGCGCCGCGGCCTCCTAAGAAGAAGCGGAAAGTCGAATTCGTGGATCTGCGA
ACACTGGGCTATAGCCAGCAGCAGCAGGAGAAGATCAAACCCAAGGTGAGGTC
CACAGTCGCACAGCACCATGAAGCCCTGGTGGGCCACGGGTTCACTCACGCTCA
TATTGTCGCACTGTCTCAGCATCCAGCCGCTCTGGGAACCGTGGCAGTCACATAC
CAGCACATCATTACTGCCCTGCCCGAGGCTACCCATGAAGACATCGTGGGAGTC
GGCAAACAGTGGAGCGGCGCACGGGCCCTGGAGGCTCTGCTGACCGACGCAGG
GGAACTGAGAGGACCCCCTCTGCAGCTGGATACAGGGCAGCTGGTGAAGATTGC
TAAGAGGGGAGGGGTGACAGCAATGGAAGCCGTCCACGCAAGCAGGAACGCAC
TGACAGGGGCCCCCCTGAACCTGACCCCGGACCAAGTGGTGGCTATCGCCAGCA
ACAATGGCGGCAAGCAAGCGCTCGAAACGGTGCAGCGGCTGTTGCCGGTGCTGT
GCCAGGACCATGGCCTGACCCCGGACCAAGTGGTGGCTATCGCCAGCAACAATG
GCGGCAAGCAAGCGCTCGAAACGGTGCAGCGGCTGTTGCCGGTGCTGTGCCAGG
ACCATGGCCTGACCCCGGACCAAGTGGTGGCTATCGCCAGCAACGGTGGCGGCA
AGCAAGCGCTCGAAACGGTGCAGCGGCTGTTGCCGGTGCTGTGCCAGGACCATG
GCCTGACCCCGGACCAAGTGGTGGCTATCGCCAGCCACGATGGCGGCAAGCAAG
CGCTCGAAACGGTGCAGCGGCTGTTGCCGGTGCTGTGCCAGGACCATGGCCTGA
CTCCGGACCAAGTGGTGGCTATCGCCAGCCACGATGGCGGCAAGCAAGCGCTCG
AAACGGTGCAGCGGCTGTTGCCGGTGCTGTGCCAGGACCATGGCCTGACCCCGG
ACCAAGTGGTGGCTATCGCCAGCAACGGTGGCGGCAAGCAAGCGCTCGAAACG
GTGCAGCGGCTGTTGCCGGTGCTGTGCCAGGACCATGGCCTGACTCCGGACCAA
GTGGTGGCTATCGCCAGCAACGGTGGCGGCAAGCAAGCGCTCGAAACGGTGCAG
CGGCTGTTGCCGGTGCTGTGCCAGGACCATGGCCTGACCCCGGACCAAGTGGTG
GCTATCGCCAGCCACGATGGCGGCAAGCAAGCGCTCGAAACGGTGCAGCGGCTG
TTGCCGGTGCTGTGCCAGGACCATGGCCTGACCCCGGACCAAGTGGTGGCTATC
GCCAGCCACGATGGCGGCAAGCAAGCGCTCGAAACGGTGCAGCGGCTGTTGCCG
GTGCTGTGCCAGGACCATGGCCTGACTCCGGACCAAGTGGTGGCTATCGCCAGC
AACGGTGGCGGCAAGCAAGCGCTCGAAACGGTGCAGCGGCTGTTGCCGGTGCTG
TGCCAGGACCATGGCCTGACCCCGGACCAAGTGGTGGCTATCGCCAGCAACAAT
GGCGGCAAGCAAGCGCTCGAAACGGTGCAGCGGCTGTTGCCGGTGCTGTGCCAG
GACCATGGCCTGACCCCGGACCAAGTGGTGGCTATCGCCAGCCACGATGGCGGC
AAGCAAGCGCTCGAAACGGTGCAGCGGCTGTTGCCGGTGCTGTGCCAGGACCAT
GGCCTGACCCCGGACCAAGTGGTGGCTATCGCCAGCCACGATGGCGGCAAGCAA
GCGCTCGAAACGGTGCAGCGGCTGTTGCCGGTGCTGTGCCAGGACCATGGCCTG
ACCCCGGACCAAGTGGTGGCTATCGCCAGCAACGGTGGCGGCAAGCAAGCGCTC
GAAACGGTGCAGCGGCTGTTGCCGGTGCTGTGCCAGGACCATGGCCTGACCCCG
GACCAAGTGGTGGCTATCGCCAGCCACGATGGCGGCAAGCAAGCGCTCGAAACG
GTGCAGCGGCTGTTGCCGGTGCTGTGCCAGGACCATGGCCTGACTCCGGACCAA
GTGGTGGCTATCGCCAGCAACATTGGCGGCAAGCAAGCGCTCGAAACGGTGCAG
CGGCTGTTGCCGGTGCTGTGCCAGGACCATGGCCTGACCCCGGACCAAGTGGTG
GCTATCGCCAGCAACGGTGGCGGCAAGCAAGCGCTCGAAAGCATTGTGGCCCAG
CTGAGCCGGCCTGATCCGGCGTTGGCCGCGTTGACCAACGACCACCTGGTCGCTC
TGGCTTGCCTGGGAGGACGCCCTGCTATGGACGCTGTGAAGAAAGGACTGCCCC
ACGCACCCGAACTGATTAGACGGGTGAACCGGAGAATCGGCGAGAGAACATCC
CATAGGGTGGCAATCTCTAGAACTCAGCTGGTCAAGAGTGAACTGGAGGAAAAG
AAATCAGAGCTGCGCCACAAGCTGAAATACGTGCCTCATGAGTATATCGAACTG
ATCGAGATTGCTCGCAATTCAACCCAGGACCGGATCCTGGAAATGAAAGTGATG
GAGTTCTTTATGAAAGTCTACGGATATCGGGGGAAACACCTGGGAGGGAGCAGA
AAGCCAGATGGGGCCATCTACACAGTGGGATCCCCCATCGACTATGGCGTGATT
GTCGATACTAAAGCCTACAGCGGAGGCTATAACCTGCCTATCGGCCAGGCTGAC
GAGATGCAGAGATACGTGGAGGAAAACCAGACCCGCAATAAGCATATTAACCC
CAATGAATGGTGGAAAGTGTATCCTAGCTCCGTCACAGAGTTCAAGTTTCTGTTC
GTGAGCGGACACTTTAAGGGCAACTACAAAGCACAGCTGACTAGGCTGAATCAT
ATCACCAACTGCAATGGAGCCGTGCTGTCTGTCGAGGAACTGCTGATCGGGGGA
GAGATGATTAAGGCTGGCACACTGACTCTGGAGGAAGTGAGGCGCAAGTTCAAC
AATGGGGAAATCAACTTCTAA
SEQ ID NO: 26 #1380 AAV.WASATGcoWAS.WPRE3.pA
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTAGGCTCGTCTTGAACTCCTGACCTCAAGTGAT
CCACTCGTCTCGGCCTCCCAAAGTGCTGGGATTACAGGTGTGAGCTATTGTCCCC
AGCCAAAAGGAAAAGTTTTACTGTAGTAACCCTTCCGGACTAGGGACCTCGGGC
CTCAGCCTCAGGCTACCTAGGTGCTTTAGAAAGGAGGCCACCCAGGCCCATGAC
TACTCCTTGCCACAGGGAGCCCTGCACACAGATGTGCTAAGCTCTCGCTGCCAGC
CAGAGGGAGGAGGGTCTGAGCCAGTCAGAAGGAGATGGGCCCCAGAGAGTAAG
AAAGGGGGAGGAGGACCCAAGCTGATCCAAAAGGTGGGTCTAAGCAGTCAAGT
GGAGGAGGGTTCCAATCTGATGGCGGAGGGCCCAAGCTCAGCCTAACGAGGAG
GCCAGGCCCACCAAGGGGCCCCTGGAGGACTTGTTTCCCTTGTCCCTTGTGGTTT
TTTGCATTTCCTGTTCCCTTGCTGCTCATTGCGGAAGTTCCTCTTCTTACCCTGCA
CCCAGAGCCTCGCCAGAGAAGACAAGGGCAGAAAGCACCATGGGAGGAAGACC
CGGCGGCCGAGGAGCGCCAGCAGTGCAACAAAACATTCCGTCAACCCTGCTGCA
GGACCACGAAAACCAGAGGCTGTTTGAAATGTTGGGACGGAAGTGTCTCACTCT
CGCCACAGCCGTCGTCCAGCTTTATCTTGCGCTTCCTCCCGGTGCTGAGCATTGG
ACTAAAGAGCATTGCGGCGCGGTCTGTTTTGTCAAGGATAATCCCCAAAAATCAT
ATTTCATTAGGTTGTACGGACTCCAAGCTGGACGCCTTCTGTGGGAACAAGAACT
CTATAGCCAGCTCGTATATAGCACACCGACCCCTTTCTTCCATACTTTCGCGGGA
GACGACTGTCAGGCGGGCTTGAACTTTGCGGACGAGGATGAAGCTCAGGCTTTC
CGAGCATTGGTTCAAGAAAAAATCCAGAAAAGAAATCAGCGACAGTCCGGAGA
TCGCCGGCAGCTGCCGCCGCCACCTACACCGGCCAATGAGGAACGGAGGGGAG
GCCTTCCGCCACTTCCATTGCATCCAGGCGGCGATCAGGGTGGGCCACCAGTAG
GGCCCTTGAGTTTGGGTCTCGCTACTGTGGATATACAGAACCCGGACATAACATC
TAGCCGCTACCGCGGACTGCCGGCTCCAGGTCCGTCCCCCGCTGATAAAAAGCG
CTCCGGCAAAAAGAAGATATCTAAAGCAGATATCGGTGCGCCCTCCGGTTTCAA
GCATGTCTCCCATGTAGGATGGGACCCGCAAAATGGATTCGACGTTAATAACCTC
GATCCGGACCTGAGGAGTCTCTTCTCTCGCGCGGGTATCAGCGAGGCACAGCTT
ACTGATGCCGAAACAAGTAAGTTGATATACGACTTTATCGAGGATCAAGGAGGG
CTGGAAGCGGTCAGGCAAGAAATGCGGCGACAAGAACCTTTGCCCCCGCCCCCG
CCCCCGTCCAGAGGCGGGAACCAGCTTCCACGCCCACCTATCGTTGGAGGGAAT
AAAGGCAGGTCTGGGCCACTCCCTCCGGTACCGTTGGGGATCGCTCCACCGCCTC
CTACGCCTAGGGGACCCCCGCCTCCTGGTCGGGGGGGACCGCCCCCTCCGCCGC
CTCCAGCCACTGGTCGAAGTGGACCCCTCCCGCCTCCTCCACCCGGCGCCGGGG
GCCCACCGATGCCACCTCCTCCTCCGCCCCCACCGCCTCCCCCTTCTTCCGGCAA
CGGTCCCGCACCTCCGCCCCTCCCTCCGGCATTGGTCCCCGCGGGGGGCCTCGCG
CCTGGTGGTGGCCGGGGTGCACTTCTGGATCAAATCCGACAGGGCATACAGTTG
AATAAGACGCCCGGCGCCCCTGAAAGCTCAGCTCTGCAACCGCCGCCTCAGTCC
TCTGAAGGGTTGGTAGGCGCGCTCATGCATGTAATGCAGAAGCGCAGTCGCGCT
ATCCACTCATCAGATGAAGGTGAAGACCAGGCCGGTGACGAGGACGAAGACGA
TGAATGGGACGATTGACTGAACTGAACTAGTGTCGACGATAATCAACCTCTGGA
TTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCT
TTCATTTTCTCCTCCTTGTATAAATCCTGGTTAGTTCTTGCCACGGCGGAACTCAT
CGCCGCCTGCCTTGCCCGCTGCTGGACAGGGGCTCGGCTGTTGGGCACTGACAAT
TCCGTGGGTCGACTGCTTTATTTGTGAAATTTGTGATGCTATTGCTTTATTTGTAA
CCATTATAAGCTGCAATAAACAAGTTAACAACAACAATTGCATTCATTTTATGTT
TCAGGTTCAGGGGGAGGTGTGGGAGGTTTTTTAAACAGCGGTTCAGCAGAACAT
ACCCTCCACCCTCCTCCAGGACCACGAGAACCAGCGACTCTTTGAGATGCTTGGA
CGAAAATGCTTGGTGAGCTGGGGATCTCCTGCCCCCGCCCCGTCCCCACCGTTTC
TTCCTCTTCCTCTCCTCCTTCTCTCTCTTCCCCTCCTCCCGCTCCTCCTTTCCCTCTC
CATCATCTCCTCTCCTAGAATTTCCCGTCATAATCCACCCTTCCCAGGAAGATCTC
AATGTCTACTTGCCTTCCCTCTGGCTGCAGCTCTTCCTTTGGGCCCATGACTGTCA
TGAGGCAGGAAGGACCAGGTCTGGCTCCAAGACCTTGTGGCTACCCCTGACCAG
ACTCCACTGACCCCTGCTTTCCTCTCCCAGACGCTGGCCACTGCAGTTGTTCAGC
TGTACCTGGCGCTGCCCCCTGGAGCTGAGCACTGGACCAAGGAGCATTGTGGGG
CTGTGTGCTTCGTGAAGGATAACCCCCAGAAGTCCTACTTCATCCGCCTTTACGG
CCTTCAGGTGACCCCCCCACCCCCGACTGGACTTGCAAGCCAGTTCTCAACCCGC
AAACCCAGATCTGTGTCCATATGTGTCCATAGCTTCAAGTCTAGAGCATGGCTAC
GTAGATAAGTAGCATGGCGGGTTAATCATTAACTACAAGGAACCCCTAGTGATG
GAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGC
GCCAGCTGGCGTAATAGCGAAGAGGCCCGCACCGATCGCCCTTCCCAACAGTTG
CGCAGCCTGAATGGCGAATGGCGATTCCGTTGCAATGGCTGGCGGTAATATTGTT
CTGGATATTACCAGCAAGGCCGATAGTTTGAGTTCTTCTACTCAGGCAAGTGATG
TTATTACTAATCAAAGAAGTATTGCGACAACGGTTAATTTGCGTGATGGACAGAC
TCTTTTACTCGGTGGCCTCACTGATTATAAAAACACTTCTCAGGATTCTGGCGTA
CCGTTCCTGTCTAAAATCCCTTTAATCGGCCTCCTGTTTAGCTCCCGCTCTGATTC
TAACGAGGAAAGCACGTTATACGTGCTCGTCAAAGCAACCATAGTACGCGCCCT
GTAGCGGCGCATTAAGCGCGGCGGGTGTGGTGGTTACGCGCAGCGTGACCGCTA
CACTTGCCAGCGCCCTAGCGCCCGCTCCTTTCGCTTTCTTCCCTTCCTTTCTCGCC
ACGTTCGCCGGCTTTCCCCGTCAAGCTCTAAATCGGGGGCTCCCTTTAGGGTTCC
GATTTAGTGCTTTACGGCACCTCGACCCCAAAAAACTTGATTAGGGTGATGGTTC
ACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAA
AATGAGCTGATTTAACAAAAATTTAACGCGAATTTTAACAAAATATTAACGTTTA
CAATTTAAATATTTGCTTATACAATCTTCCTGTTTTTGGGGCTTTTCTGATTATCA
ACCGGGGTACATATGATTGACATGCTAGTTTTACGATTACCGTTCATCGATTCTC
TTGTTTGCTCCAGACTCTCAGGCAATGACCTGATAGCCTTTGTAGAGACCTCTCA
AAAATAGCTACCCTCTCCGGCATGAATTTATCAGCTAGAACGGTTGAATATCATA
TTGATGGTGATTTGACTGTCTCCGGCCTTTCTCACCCGTTTGAATCTTTACCTACA
CATTACTCAGGCATTGCATTTAAAATATATGAGGGTTCTAAAAATTTTTATCCTT
GCGTTGAAATAAAGGCTTCTCCCGCAAAAGTATTACAGGGTCATAATGTTTTTGG
TACAACCGATTTAGCTTTATGCTCTGAGGCTTTATTGCTTAATTTTGCTAATTCTT
TGCCTTGCCTGTATGATTTATTGGATGTTGGAATCGCCTGATGCGGTATTTTCTCC
TTACGCATCTGTGCGGTATTTCACACCGCATATGGTGCACTCTCAGTACAATCTG
CTCTGATGCCGCATAGTTAAGCCAGCCCCGACACCCGCCAACACCCGCTGACGC
GCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGA
CGAAAGGGCCTCGTGATACGCCTATTTTTATAGGTTAATGTCATGATAATAATGG
TTTCTTAGACGTCAGGTGGCACTTTTCGGGGAAATGTGCGCGGAACCCCTATTTG
TTTATTTTTCTAAATACATTCAAATATGTATCCGCTCATGAGACAATAACCCTGAT
AAATGCTTCAATAATATTGAAAAAGGAAGAGTATGAGTATTCAACATTTCCGTGT
CGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACA
TCGAACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTATCCCGT
ATTGACGCCGGGCAAGAGCAACTCGGTCGCCGCATACACTATTCTCAGAATGAC
TTGGTTGAGTACTCACCAGTCACAGAAAAGCATCTTACGGATGGCATGACAGTA
AGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATA
CCAAACGACGAGCGTGACACCACGATGCCTGTAGCAATGGCAACAACGTTGCGC
AAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTG
CCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAAACC
GCCTCTCCCCGCGCGTTGGCCGATTCATTAATG
SEQ ID NO: 27 WAS TALEN#1forward RVD sequence
HD NG HD HD NG NI NN NI NI NG NG NG HD HD HD NN NG
| | | | | | | | | | | | | | | | |
C T C C T A G A A T T T C C C G T
SEQ ID NO: 28 WAS TALEN#1 reverse RVD sequence
NI NN NI HD NI NG NG NN NI NN NI NG HD NG NG HD HD NG
| | | | | | | | | | | | | | | | | I
A G A C A T T G A G A T C T T C C T
SEQ ID NO: 29 WAS TALEN#2 forward RVD sequence
HD NG NN NN HD NG NN HD NI NN HD NG HD NG NG HD HD NG
| | | | | | | | | | | | | | | | | I
C T G G C T G C A G C T C T T C C T
SEQ ID NO: 30 WAS TALEN#2 reverse RVD sequence
NN NN NG HD HD NG NG HD HD NG NN HD HD NG HD NI NG
| | | | | | | | | | | | | | | | |
G G T C C T T C C T G C C T C A T
SEQ ID NO: 31 WAS GUIDE#1 sequence
GGTATGTTCTGCTGAACCGC
SEQ ID NO: 32 WAS GUIDE#2 sequence
CAAAGAGTCGCTGGTTCTCG
SEQ ID NO: 33 WAS GUIDE#3 sequence
CAAGCATCTCAAAGAGTCGC
SEQ ID NO: 34 WAS GUIDE#4 sequence
ACCATGAGTGGGGGCCCAAT
SEQ ID NO: 35 pAAV.DT(#1201)
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTGTATGACAAGCAGAAAGTAATTTGGGAGCTG
CGGGGAGGCAAGGGTAAGGGATGGGGAAGTGGACCAGAGGCATATGCGTCATT
GGCAGTGTCTAAGCACTCACGATAGGCGTGGATCACAGGGGCTCGCTCTGTAAT
TAAAAGGAAAAGGGTTTTTGTTGTGTTGTTGTTGTTGCTGTTTTTGAGACAAGGG
TCTTGCTCTGTCATCATCCAGGCTGGAGTGCAGTGGTGCAGTCTCAGCTCACTGC
AACCTCCGCCTCCTGGGTTCAAGCGATTCTCCTGCCTCAGCCTCCTGAGCAGCTA
GGACTACAGGTGTGTGCCACCATGCCTGGCTAATTTTTGTATTTTTTAGTGGAAA
TGGGGTTTTGCCATGTTGCCCAGGCTCGTCTTGAACTCCTGACCTCAAGTGATCC
ACTCGTCTCGGCCTCCCAAAGTGCTGGGATTACAGGTGTGAGCTATTGTCCCCAG
CCAAAAGGAAAAGTTTTACTGTAGTAACCCTTCCGGACTAGGGACCTCGGGCCT
CAGCCTCAGGCTACCTAGGTGCTTTAGAAAGGAGGCCACCCAGGCCCATGACTA
CTCCTTGCCACAGGGAGCCCTGCACACAGATGTGCTAAGCTCTCGCTGCCAGCCA
GAGGGAGGAGGGTCTGAGCCAGTCAGAAGGAGATGGGCCCCAGAGAGTAAGAA
AGGGGGAGGAGGACCCAAGCTGATCCAAAAGGTGGGTCTAAGCAGTCAAGTGG
AGGAGGGTTCCAATCTGATGGCGGAGGGCCCAAGCTCAGCCTAACGAGGAGGCC
AGGCCCACCAAGGGGCCCCTGGAGGACTTGTTTCCCTTGTCCCTTGTGGTTTTTT
GCATTTCCTGTTCCCTTGCTGCTCATTGCGGAAGTTCCTCTTCTTACCCTGCACCC
AGAGCCTCGCCAGAGAAGACAAGGGCAGAAAGCACCATGAGTGGGGGCCCAAT
GGGAGGAAGGCCCGGGGGCCGAGGAGCACGAACAGAGAAACAGGAGAATATG
GGCCAAACAGGATATCTGTGGTAAGCAGTTCCTGCCCCGGCTCAGGGCCAAGAA
CAGTTGGAACAGCAGAATATGGGCCAAACAGGATATCTGTGGTAAGCAGTTCCT
GCCCCGGCTCAGGGCCAAGAACAGATGGTCCCCAGATGCGGTCCCGCCCTCAGC
AGTTTCTAGAGAACCATCAGATGTTTCCAGGGTGCCCCAAGGACCTGAAATGAC
CCTGTGCCTTATTTGAACTAACCAATCAGTTCGCTTCTCGCTTCTGTTCGCGCGCT
TCTGCTCCCCGAGCTCTATATAAGCAGAGCTCGTTTAGTGAACCGTCAGATCGCC
TGGAGACGCCATCCACGCTGTTTTGACTTCCATAGAAGGATCTCGAGGCCACCAT
GGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAG
CCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACA
ACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCG
ACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGA
CGAGCTGTACAAGTAAACTAGTGTCGACTGCTTTATTTGTGAAATTTGTGATGCT
ATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAACAGC
GGTTCAGCAGAACATACCCTCCACCCTCCTCCAGGACCACGAGAACCAGCGACT
CTTTGAGATGCTTGGACGAAAATGCTTGGTGAGCTGGGGATCTCCTGCCCCCGCC
CCGTCCCCACCGTTTCTTCCTCTTCCTCTCCTCCTTCTCTCTCTTCCCCTCCTCCCG
CTCCTCCTTTCCCTCTCCATCATCTCCTCTCCTAGAATTTCCCGTCATAATCCACC
CTTCCCAGGAAGATCTCAATGTCTACTTGCCTTCCCTCTGGCTGCAGCTCTTCCTT
TGGGCCCATGACTGTCATGAGGCAGGAAGGACCAGGTCTGGCTCCAAGACCTTG
TGGCTACCCCTGACCAGACTCCACTGACCCCTGCTTTCCTCTCCCAGACGCTGGC
CACTGCAGTTGTTCAGCTGTACCTGGCGCTGCCCCCTGGAGCTGAGCACTGGACC
AAGGAGCATTGTGGGGCTGTGTGCTTCGTGAAGGATAACCCCCAGAAGTCCTAC
TTCATCCGCCTTTACGGCCTTCAGGTGACCCCCCCACCCCCGACTGGACTTGCAA
GCCAGTTCTCAACCCGCAAACCCAGATCTGTGTCCATATGTGTCCATAGCTTCAA
GACCTCAGACCTGATCAGTGAATCCCTGAGCCCCAGAACCAAAGACTCATCCAG
ATGGCAAACTCTGACTTGCCTTTCTAAGTCTGCAATGACTGGCCCCAGTCTCCGT
ATCAAGATCTCTAAAGCCCCCAGTATTAGTCTGCTGCCTAAGCCTAATCTTTTCC
ACAAATTCCAATAAATGAGCACTGTATTTGTACCTGAACCTCAAATCTATTCTAA
ACTCAACATTTTGCATCCCAGGAATCTCTCATCAAAACTCCTGAACCCCAGATGT
TTGCCAAGCTCCTAAGTCATAAATCTGTTCAACAAACCCCAAAGTTGAATATTCC
ATTGATCCTTGAACTCCAAATCTGTCCTTCTAAATCCACAGCACAGACCCCAGAG
TTCCCATCTAGAGCATGGCTACGTAGATAAGTAGCATGGCGGGTTAATCATTAAC
TACAAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGG
CCTCAGTGAGCGAGCGAGCGCGCCAGCTGGCGTAATAGCGAAGAGGCCCGCACC
GATCGCCCTTCCCAACAGTTGCGCAGCCTGAATGGCGAATGGCGATTCCGTTGCA
ATGGCTGGCGGTAATATTGTTCTGGATATTACCAGCAAGGCCGATAGTTTGAGTT
CTTCTACTCAGGCAAGTGATGTTATTACTAATCAAAGAAGTATTGCGACAACGGT
TAATTTGCGTGATGGACAGACTCTTTTACTCGGTGGCCTCACTGATTATAAAAAC
ACTTCTCAGGATTCTGGCGTACCGTTCCTGTCTAAAATCCCTTTAATCGGCCTCCT
GTTTAGCTCCCGCTCTGATTCTAACGAGGAAAGCACGTTATACGTGCTCGTCAAA
GCAACCATAGTACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGG
AACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTT
TAACAAAATATTAACGTTTACAATTTAAATATTTGCTTATACAATCTTCCTGTTTT
TGGGGCTTTTCTGATTATCAACCGGGGTACATATGATTGACATGCTAGTTTTACG
ATTACCGTTCATCGATTCTCTTGTTTGCTCCAGACTCTCAGGCAATGACCTGATAG
CCTTTGTAGAGACCTCTCAAAAATAGCTACCCTCTCCGGCATGAATTTATCAGCT
AGAACGGTTGAATATCATATTGATGGTGATTTGACTGTCTCCGGCCTTTCTCACC
CGTTTGAATCTTTACCTACACATTACTCAGGCATTGCATTTAAAATATATGAGGG
TTCTAAAAATTTTTATCCTTGCGTTGAAATAAAGGCTTCTCCCGCAAAAGTATTA
CAGGGTCATAATGTTTTTGGTACAACCGATTTAGCTTTATGCTCTGAGGCTTTATT
GCTTAATTTTGCTAATTCTTTGCCTTGCCTGTATGATTTATTGGATGTTGGAATCG
CCTGATGCGGTATTTTCTCCTTACGCATCTGTGCGGTATTTCACACCGCATATGGT
GCACTCTCAGTACAATCTGCTCTGATGCCGCATAGTTAAGCCAGCCCCGACACCC
GCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCAT
CACCGAAACGCGCGAGACGAAAGGGCCTCGTGATACGCCTATTTTTATAGGTTA
ATGTCATGATAATAATGGTTTCTTAGACGTCAGGTGGCACTTTTCGGGGAAATGT
GCGCGGAACCCCTATTTGTTTATTTTTCTAAATACATTCAAATATGTATCCGCTCA
TGAGACAATAACCCTGATAAATGCTTCAATAATATTGAAAAAGGAAGAGTATGA
GTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGG
GTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATG
TGGCGCGGTATTATCCCGTATTGACGCCGGGCAAGAGCAACTCGGTCGCCGCAT
ACACTATTCTCAGAATGACTTGGTTGAGTACTCACCAGTCACAGAAAAGCATCTT
ACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGG
AGCTGAATGAAGCCATACCAAACGACGAGCGTGACACCACGATGCCTGTAGCAA
TGGCAACAACGTTGCGCAAACTATTAACTGGCGAACTACTTACTCTAGCTTCCCG
GCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGT
GGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCA
GCAGAGCGCAGATACCAAATACTGTCCTTCTAGTGTAGCCGTAGTTAGGCCACC
ACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATG
SEQ ID NO: 36 pAAV.DT-M (#1244)
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTGTATGACAAGCAGAAAGTAATTTGGGAGCTG
CGGGGAGGCAAGGGTAAGGGATGGGGAAGTGGACCAGAGGCATATGCGTCATT
GGCAGTGTCTAAGCACTCACGATAGGCGTGGATCACAGGGGCTCGCTCTGTAAT
TAAAAGGAAAAGGGTTTTTGTTGTGTTGTTGTTGTTGCTGTTTTTGAGACAAGGG
TCTTGCTCTGTCATCATCCAGGCTGGAGTGCAGTGGTGCAGTCTCAGCTCACTGC
AACCTCCGCCTCCTGGGTTCAAGCGATTCTCCTGCCTCAGCCTCCTGAGCAGCTA
GGACTACAGGTGTGTGCCACCATGCCTGGCTAATTTTTGTATTTTTTAGTGGAAA
TGGGGTTTTGCCATGTTGCCCAGGCTCGTCTTGAACTCCTGACCTCAAGTGATCC
ACTCGTCTCGGCCTCCCAAAGTGCTGGGATTACAGGTGTGAGCTATTGTCCCCAG
CCAAAAGGAAAAGTTTTACTGTAGTAACCCTTCCGGACTAGGGACCTCGGGCCT
CAGCCTCAGGCTACCTAGGTGCTTTAGAAAGGAGGCCACCCAGGCCCATGACTA
CTCCTTGCCACAGGGAGCCCTGCACACAGATGTGCTAAGCTCTCGCTGCCAGCCA
GAGGGAGGAGGGTCTGAGCCAGTCAGAAGGAGATGGGCCCCAGAGAGTAAGAA
AGGGGGAGGAGGACCCAAGCTGATCCAAAAGGTGGGTCTAAGCAGTCAAGTGG
AGGAGGGTTCCAATCTGATGGCGGAGGGCCCAAGCTCAGCCTAACGAGGAGGCC
AGGCCCACCAAGGGGCCCCTGGAGGACTTGTTTCCCTTGTCCCTTGTGGTTTTTT
GCATTTCCTGTTCCCTTGCTGCTCATTGCGGAAGTTCCTCTTCTTACCCTGCACCC
AGAGCCTCGCCAGAGAAGACAAGGGCAGAAAGCACCATGAGTGGGGGCCCAAT
GGGAGGAAGGCCCGGGGGCCGAGGAGCACGAACAGAGAAACAGGAGAATATG
GGCCAAACAGGATATCTGTGGTAAGCAGTTCCTGCCCCGGCTCAGGGCCAAGAA
CAGTTGGAACAGCAGAATATGGGCCAAACAGGATATCTGTGGTAAGCAGTTCCT
GCCCCGGCTCAGGGCCAAGAACAGATGGTCCCCAGATGCGGTCCCGCCCTCAGC
AGTTTCTAGAGAACCATCAGATGTTTCCAGGGTGCCCCAAGGACCTGAAATGAC
CCTGTGCCTTATTTGAACTAACCAATCAGTTCGCTTCTCGCTTCTGTTCGCGCGCT
TCTGCTCCCCGAGCTCTATATAAGCAGAGCTCGTTTAGTGAACCGTCAGATCGCC
TGGAGACGCCATCCACGCTGTTTTGACTTCCATAGAAGGATCTCGAGGCCACCAT
GGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAG
CCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACA
ACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCG
ACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGA
CGAGCTGTACAAGTAAACTAGTGTCGACTGCTTTATTTGTGAAATTTGTGATGCT
ATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAACAGC
GGTTCAGCAGAACATACCCTCCACCCTCCTCCAGGACCACGAGAACCAGCGACT
CTTTGAGATGCTTGGACGAAAATGCTTGGTGAGCTGGGGATCTCCTGCCCCCGCC
CCGTCCCCACCGTTTCTTCCTCTTCCTCTCCTCCTTCTCTCTCTTCCCCTCCTCCCG
CTCCTCCTTTCCCTCTCCATCATCTCCACTCCTAGAATTTCCCGTCATAATCCACC
CTTCCCAGGAAGATCTCAATGTCTTCTTGCCTTCCCTCTGGCTGCAGCTCTTCCTT
TGGGCCCATGACTGTCATGAGGCAGGAAGGACCAGGTCTGGCTCCAAGACCTTG
TGGCTACCCCTGACCAGACTCCACTGACCCCTGCTTTCCTCTCCCAGACGCTGGC
CACTGCAGTTGTTCAGCTGTACCTGGCGCTGCCCCCTGGAGCTGAGCACTGGACC
AAGGAGCATTGTGGGGCTGTGTGCTTCGTGAAGGATAACCCCCAGAAGTCCTAC
TTCATCCGCCTTTACGGCCTTCAGGTGACCCCCCCACCCCCGACTGGACTTGCAA
GCCAGTTCTCAACCCGCAAACCCAGATCTGTGTCCATATGTGTCCATAGCTTCAA
GACCTCAGACCTGATCAGTGAATCCCTGAGCCCCAGAACCAAAGACTCATCCAG
ATGGCAAACTCTGACTTGCCTTTCTAAGTCTGCAATGACTGGCCCCAGTCTCCGT
ATCAAGATCTCTAAAGCCCCCAGTATTAGTCTGCTGCCTAAGCCTAATCTTTTCC
ACAAATTCCAATAAATGAGCACTGTATTTGTACCTGAACCTCAAATCTATTCTAA
ACTCAACATTTTGCATCCCAGGAATCTCTCATCAAAACTCCTGAACCCCAGATGT
TTGCCAAGCTCCTAAGTCATAAATCTGTTCAACAAACCCCAAAGTTGAATATTCC
ATTGATCCTTGAACTCCAAATCTGTCCTTCTAAATCCACAGCACAGACCCCAGAG
TTCCCATCTAGAGCATGGCTACGTAGATAAGTAGCATGGCGGGTTAATCATTAAC
TACAAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGG
CCTCAGTGAGCGAGCGAGCGCGCCAGCTGGCGTAATAGCGAAGAGGCCCGCACC
GATCGCCCTTCCCAACAGTTGCGCAGCCTGAATGGCGAATGGCGATTCCGTTGCA
ATGGCTGGCGGTAATATTGTTCTGGATATTACCAGCAAGGCCGATAGTTTGAGTT
CTTCTACTCAGGCAAGTGATGTTATTACTAATCAAAGAAGTATTGCGACAACGGT
TAATTTGCGTGATGGACAGACTCTTTTACTCGGTGGCCTCACTGATTATAAAAAC
ACTTCTCAGGATTCTGGCGTACCGTTCCTGTCTAAAATCCCTTTAATCGGCCTCCT
GTTTAGCTCCCGCTCTGATTCTAACGAGGAAAGCACGTTATACGTGCTCGTCAAA
GCAACCATAGTACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGG
AACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTT
TAACAAAATATTAACGTTTACAATTTAAATATTTGCTTATACAATCTTCCTGTTTT
TGGGGCTTTTCTGATTATCAACCGGGGTACATATGATTGACATGCTAGTTTTACG
ATTACCGTTCATCGATTCTCTTGTTTGCTCCAGACTCTCAGGCAATGACCTGATAG
CCTTTGTAGAGACCTCTCAAAAATAGCTACCCTCTCCGGCATGAATTTATCAGCT
AGAACGGTTGAATATCATATTGATGGTGATTTGACTGTCTCCGGCCTTTCTCACC
CGTTTGAATCTTTACCTACACATTACTCAGGCATTGCATTTAAAATATATGAGGG
TTCTAAAAATTTTTATCCTTGCGTTGAAATAAAGGCTTCTCCCGCAAAAGTATTA
CAGGGTCATAATGTTTTTGGTACAACCGATTTAGCTTTATGCTCTGAGGCTTTATT
GCTTAATTTTGCTAATTCTTTGCCTTGCCTGTATGATTTATTGGATGTTGGAATCG
CCTGATGCGGTATTTTCTCCTTACGCATCTGTGCGGTATTTCACACCGCATATGGT
GCACTCTCAGTACAATCTGCTCTGATGCCGCATAGTTAAGCCAGCCCCGACACCC
GCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCAT
CACCGAAACGCGCGAGACGAAAGGGCCTCGTGATACGCCTATTTTTATAGGTTA
ATGTCATGATAATAATGGTTTCTTAGACGTCAGGTGGCACTTTTCGGGGAAATGT
GCGCGGAACCCCTATTTGTTTATTTTTCTAAATACATTCAAATATGTATCCGCTCA
TGAGACAATAACCCTGATAAATGCTTCAATAATATTGAAAAAGGAAGAGTATGA
GTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGG
GTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATG
TGGCGCGGTATTATCCCGTATTGACGCCGGGCAAGAGCAACTCGGTCGCCGCAT
ACACTATTCTCAGAATGACTTGGTTGAGTACTCACCAGTCACAGAAAAGCATCTT
ACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGG
AGCTGAATGAAGCCATACCAAACGACGAGCGTGACACCACGATGCCTGTAGCAA
TGGCAACAACGTTGCGCAAACTATTAACTGGCGAACTACTTACTCTAGCTTCCCG
GCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGT
GGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCA
GCAGAGCGCAGATACCAAATACTGTCCTTCTAGTGTAGCCGTAGTTAGGCCACC
ACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATG
SEQ ID NO: 37 pAAV.DT-D (#1262)
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTGTATGACAAGCAGAAAGTAATTTGGGAGCTG
CGGGGAGGCAAGGGTAAGGGATGGGGAAGTGGACCAGAGGCATATGCGTCATT
GGCAGTGTCTAAGCACTCACGATAGGCGTGGATCACAGGGGCTCGCTCTGTAAT
TAAAAGGAAAAGGGTTTTTGTTGTGTTGTTGTTGTTGCTGTTTTTGAGACAAGGG
TCTTGCTCTGTCATCATCCAGGCTGGAGTGCAGTGGTGCAGTCTCAGCTCACTGC
AACCTCCGCCTCCTGGGTTCAAGCGATTCTCCTGCCTCAGCCTCCTGAGCAGCTA
GGACTACAGGTGTGTGCCACCATGCCTGGCTAATTTTTGTATTTTTTAGTGGAAA
TGGGGTTTTGCCATGTTGCCCAGGCTCGTCTTGAACTCCTGACCTCAAGTGATCC
ACTCGTCTCGGCCTCCCAAAGTGCTGGGATTACAGGTGTGAGCTATTGTCCCCAG
CCAAAAGGAAAAGTTTTACTGTAGTAACCCTTCCGGACTAGGGACCTCGGGCCT
CAGCCTCAGGCTACCTAGGTGCTTTAGAAAGGAGGCCACCCAGGCCCATGACTA
CTCCTTGCCACAGGGAGCCCTGCACACAGATGTGCTAAGCTCTCGCTGCCAGCCA
GAGGGAGGAGGGTCTGAGCCAGTCAGAAGGAGATGGGCCCCAGAGAGTAAGAA
AGGGGGAGGAGGACCCAAGCTGATCCAAAAGGTGGGTCTAAGCAGTCAAGTGG
AGGAGGGTTCCAATCTGATGGCGGAGGGCCCAAGCTCAGCCTAACGAGGAGGCC
AGGCCCACCAAGGGGCCCCTGGAGGACTTGTTTCCCTTGTCCCTTGTGGTTTTTT
GCATTTCCTGTTCCCTTGCTGCTCATTGCGGAAGTTCCTCTTCTTACCCTGCACCC
AGAGCCTCGCCAGAGAAGACAAGGGCAGAAAGCACCATGAGTGGGGGCCCAAT
GGGAGGAAGGCCCGGGGGCCGAGGAGCACGAACAGAGAAACAGGAGAATATG
GGCCAAACAGGATATCTGTGGTAAGCAGTTCCTGCCCCGGCTCAGGGCCAAGAA
CAGTTGGAACAGCAGAATATGGGCCAAACAGGATATCTGTGGTAAGCAGTTCCT
GCCCCGGCTCAGGGCCAAGAACAGATGGTCCCCAGATGCGGTCCCGCCCTCAGC
AGTTTCTAGAGAACCATCAGATGTTTCCAGGGTGCCCCAAGGACCTGAAATGAC
CCTGTGCCTTATTTGAACTAACCAATCAGTTCGCTTCTCGCTTCTGTTCGCGCGCT
TCTGCTCCCCGAGCTCTATATAAGCAGAGCTCGTTTAGTGAACCGTCAGATCGCC
TGGAGACGCCATCCACGCTGTTTTGACTTCCATAGAAGGATCTCGAGGCCACCAT
GGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAG
CCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACA
ACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCG
ACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCG
ATCACATGGTCCTGCTGGAGTTCGTGACCGCCGCCGGGATCACTCTCGGCATGGA
CGAGCTGTACAAGTAAACTAGTGTCGACTGCTTTATTTGTGAAATTTGTGATGCT
ATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAAAATC
CACCCTTCCCAGGAAGATCTCAATGTCTACTTGCCTTCCCTCTGGCTGCAGCTCTT
CCTTTGGGCCCATGACTGTCATGAGGCAGGAAGGACCAGGTCTGGCTCCAAGAC
CTTGTGGCTACCCCTGACCAGACTCCACTGACCCCTGCTTTCCTCTCCCAGACGC
TGGCCACTGCAGTTGTTCAGCTGTACCTGGCGCTGCCCCCTGGAGCTGAGCACTG
GACCAAGGAGCATTGTGGGGCTGTGTGCTTCGTGAAGGATAACCCCCAGAAGTC
CTACTTCATCCGCCTTTACGGCCTTCAGGTGACCCCCCCACCCCCGACTGGACTT
GCAAGCCAGTTCTCAACCCGCAAACCCAGATCTGTGTCCATATGTGTCCATAGCT
TCAAGACCTCAGACCTGATCAGTGAATCCCTGAGCCCCAGAACCAAAGACTCAT
CCAGATGGCAAACTCTGACTTGCCTTTCTAAGTCTGCAATGACTGGCCCCAGTCT
CCGTATCAAGATCTCTAAAGCCCCCAGTATTAGTCTGCTGCCTAAGCCTAATCTT
TTCCACAAATTCCAATAAATGAGCACTGTATTTGTACCTGAACCTCAAATCTATT
CTAAACTCAACATTTTGCATCCCAGGAATCTCTCATCAAAACTCCTGAACCCCAG
ATGTTTGCCAAGCTCCTAAGTCATAAATCTGTTCAACAAACCCCAAAGTTGAATA
TTCCATTGATCCTTGAACTCCAAATCTGTCCTTCTAAATCCACAGCACAGACCCC
AGAGTTCCCATCTAGAGCATGGCTACGTAGATAAGTAGCATGGCGGGTTAATCA
TTAACTACAAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCG
GGCGGCCTCAGTGAGCGAGCGAGCGCGCCAGCTGGCGTAATAGCGAAGAGGCC
CGCACCGATCGCCCTTCCCAACAGTTGCGCAGCCTGAATGGCGAATGGCGATTC
CGTTGCAATGGCTGGCGGTAATATTGTTCTGGATATTACCAGCAAGGCCGATAGT
TTGAGTTCTTCTACTCAGGCAAGTGATGTTATTACTAATCAAAGAAGTATTGCGA
CAACGGTTAATTTGCGTGATGGACAGACTCTTTTACTCGGTGGCCTCACTGATTA
TAAAAACACTTCTCAGGATTCTGGCGTACCGTTCCTGTCTAAAATCCCTTTAATC
GGCCTCCTGTTTAGCTCCCGCTCTGATTCTAACGAGGAAAGCACGTTATACGTGC
TCGTCAAAGCAACCATAGTACGCGCCCTGTAGCGGCGCATTAAGCGCGGCGGGT
GTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCC
CAAAAAACTTGATTAGGGTGATGGTTCACGTAGTGGGCCATCGCCCTGATAGAC
GGTTTTTCGCCCTTTGACGTTGGAGTCCACGTTCTTTAATAGTGGACTCTTGTTCC
AAACTGGAACAACACTCAACCCTATCTCGGTCTATTCTTTTGATTTATAAGGGAT
TTTGCCGATTTCGGCCTATTGGTTAAAAAATGAGCTGATTTAACAAAAATTTAAC
GCGAATTTTAACAAAATATTAACGTTTACAATTTAAATATTTGCTTATACAATCTT
CCTGTTTTTGGGGCTTTTCTGATTATCAACCGGGGTACATATGATTGACATGCTA
GTTTTACGATTACCGTTCATCGATTCTCTTGTTTGCTCCAGACTCTCAGGCAATGA
CCTGATAGCCTTTGTAGAGACCTCTCAAAAATAGCTACCCTCTCCGGCATGAATT
TATCAGCTAGAACGGTTGAATATCATATTGATGGTGATTTGACTGTCTCCGGCCT
TTCTCACCCGTTTGAATCTTTACCTACACATTACTCAGGCATTGCATTTAAAATAT
ATGAGGGTTCTAAAAATTTTTATCCTTGCGTTGAAATAAAGGCTTCTCCCGCAAA
AGTATTACAGGGTCATAATGTTTTTGGTACAACCGATTTAGCTTTATGCTCTGAG
GCTTTATTGCTTAATTTTGCTAATTCTTTGCCTTGCCTGTATGATTTATTGGATGTT
GGAATCGCCTGATGCGGTATTTTCTCCTTACGCATCTGTGCGGTATTTCACACCG
CATATGGTGCACTCTCAGTACAATCTGCTCTGATGCCGCATAGTTAAGCCAGCCC
CGACACCCGCCAACACCCGCTGACGCGCCCTGACGGGCTTGTCTGCTCCCGGCAT
CCGCTTACAGACAAGCTGTGACCGTCTCCGGGAGCTGCATGTGTCAGAGGTTTTC
ACCGTCATCACCGAAACGCGCGAGACGAAAGGGCCTCGTGATACGCCTATTTTT
ATAGGTTAATGTCATGATAATAATGGTTTCTTAGACGTCAGGTGGCACTTTTCGG
GGAAATGTGCGCGGAACCCCTATTTGTTTATTTTTCTAAATACATTCAAATATGT
ATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAG
ATCAGTTGGGTGCACGAGTGGGTTACATCGAACTGGATCTCAACAGCGGTAAGA
TCCTTGAGAGTTTTCGCCCCGAAGAACGTTTTCCAATGATGAGCACTTTTAAAGT
TCTGCTATGTGGCGCGGTATTATCCCGTATTGACGCCGGGCAAGAGCAACTCGGT
CGCCGCATACACTATTCTCAGAATGACTTGGTTGAGTACTCACCAGTCACAGAAA
AGCATCTTACGGATGGCATGACAGTAAGAGAATTATGCAGTGCTGCCATAACCA
TGAGTGATAACACTGCGGCCAACTTACTTCTGACAACGATCGGAGGACCGAAGG
AGCTAACCGCTTTTTTGCACAACATGGGGGATCATGTAACTCGCCTTGATCGTTG
GGAACCGGAGCTGAATGAAGCCATACCAAACGACGAGCGTGACACCACGATGC
CTGTAGCAATGGCAACAACGTTGCGCAAACTATTAACTGGCGAACTACTTACTCT
AGCTTCCCGGCAACAATTAATAGACTGGATGGAGGCGGATAAAGTTGCAGGACC
ACTTCTGCGCTCGGCCCTTCCGGCTGGCTGGTTTATTGCTGATAAATCTGGAGCC
GGTGAGCGTGGGTCTCGCGGTATCATTGCAGCACTGGGGCCAGATGGTAAGCCC
TCCCGTATCGTAGTTATCTACACGACGGGGAGTCAGGCAACTATGGATGAACGA
AATAGACAGATCGCTGAGATAGGTGCCTCACTGATTAAGCATTGGTAACTGTCA
GACCAAGTTTACTCATATATACTTTAGATTGATTTAAAACTTCATTTTTAATTTAA
AAGGATCTAGGTGAAGATCCTTTTTGATAATCTCATGACCAAAATCCCTTAACGT
GAGTTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTA
ACTGGCTTCAGCAGAGCGCAGATACCAAATACTGTCCTTCTAGTGTAGCCGTAGT
TAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTA
TCCGGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAA
CGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTA
ATG
SEQ ID NO: 38 pscAAV.G(#1189)
AAGCTTCCCGGGGGGATCTGGGCCACTCCCTCTCTGCGCGCTCGCTCGCTCACTG
AGGCCGGGCGACCAAAGGTCGCCCGACGCCCGGGCTTTGCCCGGGCGGCCTCAG
TGAGCGAGCGAGCGCGCAGAGAGGGAGTGGCCAACTCCATCACTAGGGGTTCCT
GGAGGGGTGGAGTCGTGACCTAGGCGATTTAAATTCATGTACAAAAAAGCAGGC
TTTAAAGGAACCAATTCAGTCGACTGGATCCGGTACCAAGGTCGGGCAGGAAGA
GGGCCTATTTCCCATGATTCCTTCATATTTGCATATACGATACAAGGCTGTTAGA
GAGATAATTAGAATTAATTTGACTGTAAACACAAAGATATTAGTACAAAATACG
TGACGTAGAAAGTAATAATTTCTTGGGTAGTTTGCAGTTTTAAAATTATGTTTTA
AAATGGACTATCATATGCTTACCGTAACTTGAAAGTATTTCGATTTCTTGGCTTTA
TATATCTTGTGGAAAGGACGAAACACCGGTATGTTCTGCTGAACCGCGTTTTAGA
GCTAGAAATAGCAAGTTAAAATAAGGCTAGTCCGTTATCAACTTGAAAAAGTGG
CACCGAGTCGGTGCTTTTTTTCTAGACCCAGCTTTCTTGTACAAAGTTGGCATTAA
CTAGTCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGC
AGAGAGGGACAGATCCGGGCCCGCATGCGTCGACAATTCACTGGCCGTCGTTTT
ACAACGTCGTGACTGGGAAAACCCTGGCGTTACCCAACTTAATCGCCTTGCAGC
ACATCCCCCTTTCGCCAGCTGGCGTAATAGCGAAGAGGCCCGCACCGATCGCCC
TTCCCAACAGTTGCGCAGCCTGAATGGCGAATGGCGCCTGATGCGGTATTTTCTC
CTTACGCATCTGTGCGGTATTTCACACCGCATATGGTGCACTCTCAGTACAATCT
GCTCTGATGCCGCATAGTTAAGCCAGCCCCGACACCCGCCAACACCCGCTGACG
CGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAG
ACGAAAGGGCCTCGTGATACGCCTATTTTTATAGGTTAATGTCATGATAATAATG
GTTTCTTAGACGTCAGGTGGCACTTTTCGGGGAAATGTGCGCGGAACCCCTATTT
GTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCC
GTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAG
TAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACAT
GGGGGATCATGTAACTCGCCTTGATCGTTGGGAACCGGAGCTGAATGAAGCCAT
ACCAAACGACGAGCGTGACACCACGATGCCTGTAGCAATGGCAACAACGTTGCG
CAAACTATTAACTGGCGAACTACTTACTCTAGCTTCCCGGCAACAATTAATAGAC
TGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTG
CCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAAACC
GCCTCTCCCCGCGCGTTGGCCGATTCATTAATGCAGCTGGCACGACAGGTTTCCC
GACTGGAAAGCGGGCAGTGAGCGCAACGCAATTAATGTGAGTTAGCTCACTCAT
TAGGCACCCCAGGCTTTACACTTTATGCTTCCGGCTCGTATGTTGTGTGGAATTGT
GAGCGGATAACAATTTCACACAGGAAACAGCTATGACCATGATTACGCCAAGCT
CTCGAGATCTAGA
SEQ ID NO: 39 pAAV.DTG(#1215)
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTTGTACAAAAAAGCAGGCTTTAAAGGAACCAA
TTCAGTCGACTGGATCCGGTACCAAGGTCGGGCAGGAAGAGGGCCTATTTCCCA
TGATTCCTTCATATTTGCATATACGATACAAGGCTGTTAGAGAGATAATTAGAAT
TAATTTGACTGTAAACACAAAGATATTAGTACAAAATACGTGACGTAGAAAGTA
ATAATTTCTTGGGTAGTTTGCAGTTTTAAAATTATGTTTTAAAATGGACTATCATA
TGCTTACCGTAACTTGAAAGTATTTCGATTTCTTGGCTTTATATATCTTGTGGAAA
GGACGAAACACCGGTATGTTCTGCTGAACCGCGTTTTAGAGCTAGAAATAGCAA
GTTAAAATAAGGCTAGTCCGTTATCAACTTGAAAAAGTGGCACCGAGTCGGTGC
TTTTTTACGCGTGTATGACAAGCAGAAAGTAATTTGGGAGCTGCGGGGAGGCAA
GGGTAAGGGATGGGGAAGTGGACCAGAGGCATATGCGTCATTGGCAGTGTCTAA
GCACTCACGATAGGCGTGGATCACAGGGGCTCGCTCTGTAATTAAAAGGAAAAG
GGTTTTTGTTGTGTTGTTGTTGTTGCTGTTTTTGAGACAAGGGTCTTGCTCTGTCA
TCATCCAGGCTGGAGTGCAGTGGTGCAGTCTCAGCTCACTGCAACCTCCGCCTCC
TGGGTTCAAGCGATTCTCCTGCCTCAGCCTCCTGAGCAGCTAGGACTACAGGTGT
GTGCCACCATGCCTGGCTAATTTTTGTATTTTTTAGTGGAAATGGGGTTTTGCCAT
GTTGCCCAGGCTCGTCTTGAACTCCTGACCTCAAGTGATCCACTCGTCTCGGCCT
CCCAAAGTGCTGGGATTACAGGTGTGAGCTATTGTCCCCAGCCAAAAGGAAAAG
TTTTACTGTAGTAACCCTTCCGGACTAGGGACCTCGGGCCTCAGCCTCAGGCTAC
CTAGGTGCTTTAGAAAGGAGGCCACCCAGGCCCATGACTACTCCTTGCCACAGG
GAGCCCTGCACACAGATGTGCTAAGCTCTCGCTGCCAGCCAGAGGGAGGAGGGT
CTGAGCCAGTCAGAAGGAGATGGGCCCCAGAGAGTAAGAAAGGGGGAGGAGGA
CCCAAGCTGATCCAAAAGGTGGGTCTAAGCAGTCAAGTGGAGGAGGGTTCCAAT
CTGATGGCGGAGGGCCCAAGCTCAGCCTAACGAGGAGGCCAGGCCCACCAAGG
GGCCCCTGGAGGACTTGTTTCCCTTGTCCCTTGTGGTTTTTTGCATTTCCTGTTCC
CTTGCTGCTCATTGCGGAAGTTCCTCTTCTTACCCTGCACCCAGAGCCTCGCCAG
AGAAGACAAGGGCAGAAAGCACCATGAGTGGGGGCCCAATGGGAGGAAGGCCC
GGGGGCCGAGGAGCACGAACAGAGAAACAGGAGAATATGGGCCAAACAGGATA
TCTGTGGTAAGCAGTTCCTGCCCCGGCTCAGGGCCAAGAACAGTTGGAACAGCA
GAATATGGGCCAAACAGGATATCTGTGGTAAGCAGTTCCTGCCCCGGCTCAGGG
CCAAGAACAGATGGTCCCCAGATGCGGTCCCGCCCTCAGCAGTTTCTAGAGAAC
CATCAGATGTTTCCAGGGTGCCCCAAGGACCTGAAATGACCCTGTGCCTTATTTG
AACTAACCAATCAGTTCGCTTCTCGCTTCTGTTCGCGCGCTTCTGCTCCCCGAGCT
CTATATAAGCAGAGCTCGTTTAGTGAACCGTCAGATCGCCTGGAGACGCCATCC
ACGCTGTTTTGACTTCCATAGAAGGATCTCGAGGCCACCATGGTGAGCAAGGGC
GAGGAGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGG
AGCGCACCATCTTCTTCAAGGACGACGGCAACTACAAGACCCGCGCCGAGGTGA
AGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCAC
AACGTCTATATCATGGCCGACAAGCAGAAGAACGGCATCAAGGTGAACTTCAAG
ATCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAGC
ACCCAGTCCGCCCTGAGCAAAGACCCCAACGAGAAGCGCGATCACATGGTCCTG
CTGGAGTTCGTGACCGCCGCCGGGATCACTCTCGGCATGGACGAGCTGTACAAG
TAAACTAGTGTCGACTGCTTTATTTGTGAAATTTGTGATGCTATTGCTTTATTTGT
AACCATTATAAGCTGCAATAAACAAGTTAACAACAACAATTGCATTCATTTTATG
TTTCAGGTTCAGGGGGAGGTGTGGGAGGTTTTTTAAACAGCGGTTCAGCAGAAC
ATACCCTCCACCCTCCTCCAGGACCACGAGAACCAGCGACTCTTTGAGATGCTTG
GACGAAAATGCTTGGTGAGCTGGGGATCTCCTGCCCCCGCCCCGTCCCCACCGTT
TCTTCCTCTTCCTCTCCTCCTTCTCTCTCTTCCCCTCCTCCCGCTCCTCCTTTCCCTC
TCCATCATCTCCTCTCCTAGAATTTCCCGTCATAATCCACCCTTCCCAGGAAGATC
TCAATGTCTACTTGCCTTCCCTCTGGCTGCAGCTCTTCCTTTGGGCCCATGACTGT
CATGAGGCAGGAAGGACCAGGTCTGGCTCCAAGACCTTGTGGCTACCCCTGACC
AGACTCCACTGACCCCTGCTTTCCTCTCCCAGACGCTGGCCACTGCAGTTGTTCA
GCTGTACCTGGCGCTGCCCCCTGGAGCTGAGCACTGGACCAAGGAGCATTGTGG
GGCTGTGTGCTTCGTGAAGGATAACCCCCAGAAGTCCTACTTCATCCGCCTTTAC
GGCCTTCAGGTGACCCCCCCACCCCCGACTGGACTTGCAAGCCAGTTCTCAACCC
GCAAACCCAGATCTGTGTCCATATGTGTCCATAGCTTCAAGACCTCAGACCTGAT
CAGTGAATCCCTGAGCCCCAGAACCAAAGACTCATCCAGATGGCAAACTCTGAC
TTGCCTTTCTAAGTCTGCAATGACTGGCCCCAGTCTCCGTATCAAGATCTCTAAA
GCCCCCAGTATTAGTCTGCTGCCTAAGCCTAATCTTTTCCACAAATTCCAATAAA
TGAGCACTGTATTTGTACCTGAACCTCAAATCTATTCTAAACTCAACATTTTGCAT
CCCAGGAATCTCTCATCAAAACTCCTGAACCCCAGATGTTTGCCAAGCTCCTAAG
TCATAAATCTGTTCAACAAACCCCAAAGTTGAATATTCCATTGATCCTTGAACTC
CAAATCTGTCCTTCTAAATCCACAGCACAGACCCCAGAGTTCCCATCTAGAGCAT
GGCTACGTAGATAAGTAGCATGGCGGGTTAATCATTAACTACAAGGAACCCCTA
GTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGC
GAGCGCGCCAGCTGGCGTAATAGCGAAGAGGCCCGCACCGATCGCCCTTCCCAA
CAGTTGCGCAGCCTGAATGGCGAATGGCGATTCCGTTGCAATGGCTGGCGGTAA
TATTGTTCTGGATATTACCAGCAAGGCCGATAGTTTGAGTTCTTCTACTCAGGCA
AGTGATGTTATTACTAATCAAAGAAGTATTGCGACAACGGTTAATTTGCGTGATG
GACAGACTCTTTTACTCGGTGGCCTCACTGATTATAAAAACACTTCTCAGGATTC
TGGCGTACCGTTCCTGTCTAAAATCCCTTTAATCGGCCTCCTGTTTAGCTCCCGCT
CTGATTCTAACGAGGAAAGCACGTTATACGTGCTCGTCAAAGCAACCATAGTAC
GCGCCCTGTAGCGGCGCATTAAGCGCGGCGGGTGTGGTGGTTACGCGCAGCGTG
ACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTA
GGGTTCCGATTTAGTGCTTTACGGCACCTCGACCCCAAAAAACTTGATTAGGGTG
ATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAAC
CCTATCTCGGTCTATTCTTTTGATTTATAAGGGATTTTGCCGATTTCGGCCTATTG
GTTAAAAAATGAGCTGATTTAACAAAAATTTAACGCGAATTTTAACAAAATATT
AACGTTTACAATTTAAATATTTGCTTATACAATCTTCCTGTTTTTGGGGCTTTTCT
GATTATCAACCGGGGTACATATGATTGACATGCTAGTTTTACGATTACCGTTCAT
CGATTCTCTTGTTTGCTCCAGACTCTCAGGCAATGACCTGATAGCCTTTGTAGAG
ACCTCTCAAAAATAGCTACCCTCTCCGGCATGAATTTATCAGCTAGAACGGTTGA
ATATCATATTGATGGTGATTTGACTGTCTCCGGCCTTTCTCACCCGTTTGAATCTT
TACCTACACATTACTCAGGCATTGCATTTAAAATATATGAGGGTTCTAAAAATTT
TTATCCTTGCGTTGAAATAAAGGCTTCTCCCGCAAAAGTATTACAGGGTCATAAT
GTTTTTGGTACAACCGATTTAGCTTTATGCTCTGAGGCTTTATTGCTTAATTTTGC
TAATTCTTTGCCTTGCCTGTATGATTTATTGGATGTTGGAATCGCCTGATGCGGTA
TTTTCTCCTTACGCATCTGTGCGGTATTTCACACCGCATATGGTGCACTCTCAGTA
CAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGT
GACCGTCTCCGGGAGCTGCATGTGTCAGAGGTTTTCACCGTCATCACCGAAACGC
GCGAGACGAAAGGGCCTCGTGATACGCCTATTTTTATAGGTTAATGTCATGATAA
TAATGGTTTCTTAGACGTCAGGTGGCACTTTTCGGGGAAATGTGCGCGGAACCCC
TATTTGTTTATTTTTCTAAATACATTCAAATATGTATCCGCTCATGAGACAATAAC
CCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCC
AGAAACGCTGGTGAAAGTAAAAGATGCTGAAGATCAGTTGGGTGCACGAGTGG
GTTACATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGTATTA
TCCCGTATTGACGCCGGGCAAGAGCAACTCGGTCGCCGCATACACTATTCTCAG
AATGACTTGGTTGAGTACTCACCAGTCACAGAAAAGCATCTTACGGATGGCATG
ACAGTAAGAGAATTATGCAGTGCTGCCATAACCATGAGTGATAACACTGCGGCC
AACTTACTTCTGACAACGATCGGAGGACCGAAGGAGCTAACCGCTTTTTTGCACA
ACATGGGGGATCATGTAACTCGCCTTGATCGTTGGGAACCGGAGCTGAATGAAG
CCATACCAAACGACGAGCGTGACACCACGATGCCTGTAGCAATGGCAACAACGT
TGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATA
GGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTT
TTTGATAATCTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAG
ATACCAAATACTGTCCTTCTAGTGTAGCCGTAGTTAGGCCACCACTTCAAGAACT
CTGTAGCACCGCCTACATACCTCGCTCTGCTAATCCTGTTACCAGTGGCTGCTGC
CAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGA
GCGAACGACCTACACCGAACTGAGATACCTACAGCGTGAGCTATGAGAAAGCGC
CACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTG
GATAACCGTATTACCGCCTTTGAGTGAGCTGATACCGCTCGCCGCAGCCGAACG
ACCGAGCGCAGCGAGTCAGTGAGCGAGGAAGCGGAAGAGCGCCCAATACGCAA
ACCGCCTCTCCCCGCGCGTTGGCCGATTCATTAATG
SEQ ID NO: 40 pAAV.ATG.GFP (#1374)
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTAGGCTCGTCTTGAACTCCTGACCTCAAGTGAT
CCACTCGTCTCGGCCTCCCAAAGTGCTGGGATTACAGGTGTGAGCTATTGTCCCC
AGCCAAAAGGAAAAGTTTTACTGTAGTAACCCTTCCGGACTAGGGACCTCGGGC
CTCAGCCTCAGGCTACCTAGGTGCTTTAGAAAGGAGGCCACCCAGGCCCATGAC
TACTCCTTGCCACAGGGAGCCCTGCACACAGATGTGCTAAGCTCTCGCTGCCAGC
CAGAGGGAGGAGGGTCTGAGCCAGTCAGAAGGAGATGGGCCCCAGAGAGTAAG
AAAGGGGGAGGAGGACCCAAGCTGATCCAAAAGGTGGGTCTAAGCAGTCAAGT
GGAGGAGGGTTCCAATCTGATGGCGGAGGGCCCAAGCTCAGCCTAACGAGGAG
GCCAGGCCCACCAAGGGGCCCCTGGAGGACTTGTTTCCCTTGTCCCTTGTGGTTT
TTTGCATTTCCTGTTCCCTTGCTGCTCATTGCGGAAGTTCCTCTTCTTACCCTGCA
CCCAGAGCCTCGCCAGAGAAGACAAGGGCAGAAAGCACCATGGTGAGCAAGGG
CGAGGAGCTGTTCACCGGGGTGGTGCCCATCCTGGTCGAGCTGGACGGCGACGT
AAACGGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGG
AGCGCACCATCTTCTTCAAGGACGACGGCAACTACAAGACCCGCGCCGAGGTGA
AGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCAC
AACGTCTATATCATGGCCGACAAGCAGAAGAACGGCATCAAGGTGAACTTCAAG
ATCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAGC
ACCCAGTCCGCCCTGAGCAAAGACCCCAACGAGAAGCGCGATCACATGGTCCTG
CTGGAGTTCGTGACCGCCGCCGGGATCACTCTCGGCATGGACGAGCTGTACAAG
TAAGATAATCAACCTCTGGATTACAAAATTTGTGAAAGATTGACTGGTATTCTTA
ACTATGTTGCTCCTTTTACGCTATGTGGATACGCTGCTTTAATGCCTTTGTATCAT
GCTATTGCTTCCCGTATGGCTTTCATTTTCTCCTCCTTGTATAAATCCTGGTTAGTT
CTTGCCACGGCGGAACTCATCGCCGCCTGCCTTGCCCGCTGCTGGACAGGGGCTC
GGCTGTTGGGCACTGACAATTCCGTGGGTCGACTGCTTTATTTGTGAAATTTGTG
ATGCTATTGCTTTATTTGTAACCATTATAAGCTGCAATAAACAAGTTAACAACAA
CAATTGCATTCATTTTATGTTTCAGGTTCAGGGGGAGGTGTGGGAGGTTTTTTAA
ACAGCGGTTCAGCAGAACATACCCTCCACCCTCCTCCAGGACCACGAGAACCAG
CGACTCTTTGAGATGCTTGGACGAAAATGCTTGGTGAGCTGGGGATCTCCTGCCC
CCGCCCCGTCCCCACCGTTTCTTCCTCTTCCTCTCCTCCTTCTCTCTCTTCCCCTCC
TCCCGCTCCTCCTTTCCCTCTCCATCATCTCCTCTCCTAGAATTTCCCGTCATAAT
CCACCCTTCCCAGGAAGATCTCAATGTCTACTTGCCTTCCCTCTGGCTGCAGCTCT
TCCTTTGGGCCCATGACTGTCATGAGGCAGGAAGGACCAGGTCTGGCTCCAAGA
CCTTGTGGCTACCCCTGACCAGACTCCACTGACCCCTGCTTTCCTCTCCCAGACG
CTGGCCACTGCAGTTGTTCAGCTGTACCTGGCGCTGCCCCCTGGAGCTGAGCACT
GGACCAAGGAGCATTGTGGGGCTGTGTGCTTCGTGAAGGATAACCCCCAGAAGT
CCTACTTCATCCGCCTTTACGGCCTTCAGGTGACCCCCCCACCCCCGACTGGACT
TGCAAGCCAGTTCTCAACCCGCAAACCCAGATCTGTGTCCATATGTGTCCATAGC
TTCAAGTCTAGAGCATGGCTACGTAGATAAGTAGCATGGCGGGTTAATCATTAA
CTACAAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCG
GCCTCAGTGAGCGAGCGAGCGCGCCAGCTGGCGTAATAGCGAAGAGGCCCGCA
CCGATCGCCCTTCCCAACAGTTGCGCAGCCTGAATGGCGAATGGCGATTCCGTTG
CAATGGCTGGCGGTAATATTGTTCTGGATATTACCAGCAAGGCCGATAGTTTGAG
TTCTTCTACTCAGGCAAGTGATGTTATTACTAATCAAAGAAGTATTGCGACAACG
GTTAATTTGCGTGATGGACAGACTCTTTTACTCGGTGGCCTCACTGATTATAAAA
ACACTTCTCAGGATTCTGGCGTACCGTTCCTGTCTAAAATCCCTTTAATCGGCCTC
CTGTTTAGCTCCCGCTCTGATTCTAACGAGGAAAGCACGTTATACGTGCTCGTCA
AAGCAACCATAGTACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCG
CTTTCTTCCCTTCCTTTCTCGCCACGTTCGCCGGCTTTCCCCGTCAAGCTCTAAAT
CGGGGGCTCCCTTTAGGGTTCCGATTTAGTGCTTTACGGCACCTCGACCCCAAAA
AACTTGATTAGGGTGATGGTTCACGTAGTGGGCCATCGCCCTGATAGACGGTTTT
TCGCCCTTTGACGTTGGAGTCCACGTTCTTTAATAGTGGACTCTTGTTCCAAACTG
GAACAACACTCAACCCTATCTCGGTCTATTCTTTTGATTTATAAGGGATTTTGCCG
ATTTCGGCCTATTGGTTAAAAAATGAGCTGATTTAACAAAAATTTAACGCGAATT
TTAACAAAATATTAACGTTTACAATTTAAATATTTGCTTATACAATCTTCCTGTTT
TTGGGGCTTTTCTGATTATCAACCGGGGTACATATGATTGACATGCTAGTTTTAC
GATTACCGTTCATCGATTCTCTTGTTTGCTCCAGACTCTCAGGCAATGACCTGATA
GCCTTTGTAGAGACCTCTCAAAAATAGCTACCCTCTCCGGCATGAATTTATCAGC
TAGAACGGTTGAATATCATATTGATGGTGATTTGACTGTCTCCGGCCTTTCTCAC
CCGTTTGAATCTTTACCTACACATTACTCAGGCATTGCATTTAAAATATATGAGG
GTTCTAAAAATTTTTATCCTTGCGTTGAAATAAAGGCTTCTCCCGCAAAAGTATT
ACAGGGTCATAATGTTTTTGGTACAACCGATTTAGCTTTATGCTCTGAGGCTTTAT
TGCTTAATTTTGCTAATTCTTTGCCTTGCCTGTATGATTTATTGGATGTTGGAATC
GCCTGATGCGGTATTTTCTCCTTACGCATCTGTGCGGTATTTCACACCGCATATGG
TGCACTCTCAGTACAATCTGCTCTGATGCCGCATAGTTAAGCCAGCCCCGACACC
CGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCA
TCACCGAAACGCGCGAGACGAAAGGGCCTCGTGATACGCCTATTTTTATAGGTT
AATGTCATGATAATAATGGTTTCTTAGACGTCAGGTGGCACTTTTCGGGGAAATG
TGCGCGGAACCCCTATTTGTTTATTTTTCTAAATACATTCAAATATGTATCCGCTC
ATGAGACAATAACCCTGATAAATGCTTCAATAATATTGAAAAAGGAAGAGTATG
AGTATTCAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAGTTG
GGTGCACGAGTGGGTTACATCGAACTGGATCTCAACAGCGGTAAGATCCTTGAG
AGTTTTCGCCCCGAAGAACGTTTTCCAATGATGAGCACTTTTAAAGTTCTGCTAT
GTGGCGCGGTATTATCCCGTATTGACGCCGGGCAAGAGCAACTCGGTCGCCGCA
TACACTATTCTCAGAATGACTTGGTTGAGTACTCACCAGTCACAGAAAAGCATCT
TACGGATGGCATGACAGTAAGAGAATTATGCAGTGCTGCCATAACCATGAGTGA
TAACACTGCGGCCAACTTACTTCTGACAACGATCGGAGGACCGAAGGAGCTAAC
CGCTTTTTTGCACAACATGGGGGATCATGTAACTCGCCTTGATCGTTGGGAACCG
GAGCTGAATGAAGCCATACCAAACGACGAGCGTGACACCACGATGCCTGTAGCA
ATGGCAACAACGTTGCGCAAACTATTAACTGGCGAACTACTTACTCTAGCTTCCC
GGCAACAATTAATAGACTGGATGGAGGCGGATAAAGTTGCAGGACCACTTCTGC
GCTCGGCCCTTCCGGCTGGCTGGTTTATTGCTGATAAATCTGGAGCCGGTGAGCG
TGGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTT
CCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCA
GCAGAGCGCAGATACCAAATACTGTCCTTCTAGTGTAGCCGTAGTTAGGCCACC
ACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATG
SEQ ID NO: 41 pAAV.ATG.coWAS (#1380)
CAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTACT
TATCTACGTAGCCATGCTCTAGCGGCCTCGGCCTCTGCATAAATAAAAAAAATTA
GTCAGCCATGAGCTTGGACGCGTAGGCTCGTCTTGAACTCCTGACCTCAAGTGAT
CCACTCGTCTCGGCCTCCCAAAGTGCTGGGATTACAGGTGTGAGCTATTGTCCCC
AGCCAAAAGGAAAAGTTTTACTGTAGTAACCCTTCCGGACTAGGGACCTCGGGC
CTCAGCCTCAGGCTACCTAGGTGCTTTAGAAAGGAGGCCACCCAGGCCCATGAC
TACTCCTTGCCACAGGGAGCCCTGCACACAGATGTGCTAAGCTCTCGCTGCCAGC
CAGAGGGAGGAGGGTCTGAGCCAGTCAGAAGGAGATGGGCCCCAGAGAGTAAG
AAAGGGGGAGGAGGACCCAAGCTGATCCAAAAGGTGGGTCTAAGCAGTCAAGT
GGAGGAGGGTTCCAATCTGATGGCGGAGGGCCCAAGCTCAGCCTAACGAGGAG
GCCAGGCCCACCAAGGGGCCCCTGGAGGACTTGTTTCCCTTGTCCCTTGTGGTTT
TTTGCATTTCCTGTTCCCTTGCTGCTCATTGCGGAAGTTCCTCTTCTTACCCTGCA
CCCAGAGCCTCGCCAGAGAAGACAAGGGCAGAAAGCACCATGGGAGGAAGACC
CGGCGGCCGAGGAGCGCCAGCAGTGCAACAAAACATTCCGTCAACCCTGCTGCA
GGACCACGAAAACCAGAGGCTGTTTGAAATGTTGGGACGGAAGTGTCTCACTCT
CGCCACAGCCGTCGTCCAGCTTTATCTTGCGCTTCCTCCCGGTGCTGAGCATTGG
ACTAAAGAGCATTGCGGCGCGGTCTGTTTTGTCAAGGATAATCCCCAAAAATCAT
ATTTCATTAGGTTGTACGGACTCCAAGCTGGACGCCTTCTGTGGGAACAAGAACT
CTATAGCCAGCTCGTATATAGCACACCGACCCCTTTCTTCCATACTTTCGCGGGA
GACGACTGTCAGGCGGGCTTGAACTTTGCGGACGAGGATGAAGCTCAGGCTTTC
CGAGCATTGGTTCAAGAAAAAATCCAGAAAAGAAATCAGCGACAGTCCGGAGA
TCGCCGGCAGCTGCCGCCGCCACCTACACCGGCCAATGAGGAACGGAGGGGAG
GCCTTCCGCCACTTCCATTGCATCCAGGCGGCGATCAGGGTGGGCCACCAGTAG
GGCCCTTGAGTTTGGGTCTCGCTACTGTGGATATACAGAACCCGGACATAACATC
TAGCCGCTACCGCGGACTGCCGGCTCCAGGTCCGTCCCCCGCTGATAAAAAGCG
CTCCGGCAAAAAGAAGATATCTAAAGCAGATATCGGTGCGCCCTCCGGTTTCAA
GCATGTCTCCCATGTAGGATGGGACCCGCAAAATGGATTCGACGTTAATAACCTC
GATCCGGACCTGAGGAGTCTCTTCTCTCGCGCGGGTATCAGCGAGGCACAGCTT
ACTGATGCCGAAACAAGTAAGTTGATATACGACTTTATCGAGGATCAAGGAGGG
CTGGAAGCGGTCAGGCAAGAAATGCGGCGACAAGAACCTTTGCCCCCGCCCCCG
CCCCCGTCCAGAGGCGGGAACCAGCTTCCACGCCCACCTATCGTTGGAGGGAAT
AAAGGCAGGTCTGGGCCACTCCCTCCGGTACCGTTGGGGATCGCTCCACCGCCTC
CTACGCCTAGGGGACCCCCGCCTCCTGGTCGGGGGGGACCGCCCCCTCCGCCGC
CTCCAGCCACTGGTCGAAGTGGACCCCTCCCGCCTCCTCCACCCGGCGCCGGGG
GCCCACCGATGCCACCTCCTCCTCCGCCCCCACCGCCTCCCCCTTCTTCCGGCAA
CGGTCCCGCACCTCCGCCCCTCCCTCCGGCATTGGTCCCCGCGGGGGGCCTCGCG
CCTGGTGGTGGCCGGGGTGCACTTCTGGATCAAATCCGACAGGGCATACAGTTG
AATAAGACGCCCGGCGCCCCTGAAAGCTCAGCTCTGCAACCGCCGCCTCAGTCC
TCTGAAGGGTTGGTAGGCGCGCTCATGCATGTAATGCAGAAGCGCAGTCGCGCT
ATCCACTCATCAGATGAAGGTGAAGACCAGGCCGGTGACGAGGACGAAGACGA
TGAATGGGACGATTGACTGAACTGAACTAGTGTCGACGATAATCAACCTCTGGA
TTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCT
TTCATTTTCTCCTCCTTGTATAAATCCTGGTTAGTTCTTGCCACGGCGGAACTCAT
CGCCGCCTGCCTTGCCCGCTGCTGGACAGGGGCTCGGCTGTTGGGCACTGACAAT
TCCGTGGGTCGACTGCTTTATTTGTGAAATTTGTGATGCTATTGCTTTATTTGTAA
CCATTATAAGCTGCAATAAACAAGTTAACAACAACAATTGCATTCATTTTATGTT
TCAGGTTCAGGGGGAGGTGTGGGAGGTTTTTTAAACAGCGGTTCAGCAGAACAT
ACCCTCCACCCTCCTCCAGGACCACGAGAACCAGCGACTCTTTGAGATGCTTGGA
CGAAAATGCTTGGTGAGCTGGGGATCTCCTGCCCCCGCCCCGTCCCCACCGTTTC
TTCCTCTTCCTCTCCTCCTTCTCTCTCTTCCCCTCCTCCCGCTCCTCCTTTCCCTCTC
CATCATCTCCTCTCCTAGAATTTCCCGTCATAATCCACCCTTCCCAGGAAGATCTC
AATGTCTACTTGCCTTCCCTCTGGCTGCAGCTCTTCCTTTGGGCCCATGACTGTCA
TGAGGCAGGAAGGACCAGGTCTGGCTCCAAGACCTTGTGGCTACCCCTGACCAG
ACTCCACTGACCCCTGCTTTCCTCTCCCAGACGCTGGCCACTGCAGTTGTTCAGC
TGTACCTGGCGCTGCCCCCTGGAGCTGAGCACTGGACCAAGGAGCATTGTGGGG
CTGTGTGCTTCGTGAAGGATAACCCCCAGAAGTCCTACTTCATCCGCCTTTACGG
CCTTCAGGTGACCCCCCCACCCCCGACTGGACTTGCAAGCCAGTTCTCAACCCGC
AAACCCAGATCTGTGTCCATATGTGTCCATAGCTTCAAGTCTAGAGCATGGCTAC
GTAGATAAGTAGCATGGCGGGTTAATCATTAACTACAAGGAACCCCTAGTGATG
GAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGC
GCCAGCTGGCGTAATAGCGAAGAGGCCCGCACCGATCGCCCTTCCCAACAGTTG
CGCAGCCTGAATGGCGAATGGCGATTCCGTTGCAATGGCTGGCGGTAATATTGTT
CTGGATATTACCAGCAAGGCCGATAGTTTGAGTTCTTCTACTCAGGCAAGTGATG
TTATTACTAATCAAAGAAGTATTGCGACAACGGTTAATTTGCGTGATGGACAGAC
TCTTTTACTCGGTGGCCTCACTGATTATAAAAACACTTCTCAGGATTCTGGCGTA
CCGTTCCTGTCTAAAATCCCTTTAATCGGCCTCCTGTTTAGCTCCCGCTCTGATTC
TAACGAGGAAAGCACGTTATACGTGCTCGTCAAAGCAACCATAGTACGCGCCCT
GTAGCGGCGCATTAAGCGCGGCGGGTGTGGTGGTTACGCGCAGCGTGACCGCTA
CACTTGCCAGCGCCCTAGCGCCCGCTCCTTTCGCTTTCTTCCCTTCCTTTCTCGCC
ACGTTCGCCGGCTTTCCCCGTCAAGCTCTAAATCGGGGGCTCCCTTTAGGGTTCC
GATTTAGTGCTTTACGGCACCTCGACCCCAAAAAACTTGATTAGGGTGATGGTTC
ACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAA
AATGAGCTGATTTAACAAAAATTTAACGCGAATTTTAACAAAATATTAACGTTTA
CAATTTAAATATTTGCTTATACAATCTTCCTGTTTTTGGGGCTTTTCTGATTATCA
ACCGGGGTACATATGATTGACATGCTAGTTTTACGATTACCGTTCATCGATTCTC
TTGTTTGCTCCAGACTCTCAGGCAATGACCTGATAGCCTTTGTAGAGACCTCTCA
AAAATAGCTACCCTCTCCGGCATGAATTTATCAGCTAGAACGGTTGAATATCATA
TTGATGGTGATTTGACTGTCTCCGGCCTTTCTCACCCGTTTGAATCTTTACCTACA
CATTACTCAGGCATTGCATTTAAAATATATGAGGGTTCTAAAAATTTTTATCCTT
GCGTTGAAATAAAGGCTTCTCCCGCAAAAGTATTACAGGGTCATAATGTTTTTGG
TACAACCGATTTAGCTTTATGCTCTGAGGCTTTATTGCTTAATTTTGCTAATTCTT
TGCCTTGCCTGTATGATTTATTGGATGTTGGAATCGCCTGATGCGGTATTTTCTCC
TTACGCATCTGTGCGGTATTTCACACCGCATATGGTGCACTCTCAGTACAATCTG
CTCTGATGCCGCATAGTTAAGCCAGCCCCGACACCCGCCAACACCCGCTGACGC
GCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGA
CGAAAGGGCCTCGTGATACGCCTATTTTTATAGGTTAATGTCATGATAATAATGG
TTTCTTAGACGTCAGGTGGCACTTTTCGGGGAAATGTGCGCGGAACCCCTATTTG
TTTATTTTTCTAAATACATTCAAATATGTATCCGCTCATGAGACAATAACCCTGAT
AAATGCTTCAATAATATTGAAAAAGGAAGAGTATGAGTATTCAACATTTCCGTGT
CGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACA
TCGAACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTATCCCGT
ATTGACGCCGGGCAAGAGCAACTCGGTCGCCGCATACACTATTCTCAGAATGAC
TTGGTTGAGTACTCACCAGTCACAGAAAAGCATCTTACGGATGGCATGACAGTA
AGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATA
CCAAACGACGAGCGTGACACCACGATGCCTGTAGCAATGGCAACAACGTTGCGC
AAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTG
CCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGAT
AACCGTATTACCGCCTTTGAGTGAGCTGATACCGCTCGCCGCAGCCGAACGACC
GAGCGCAGCGAGTCAGTGAGCGAGGAAGCGGAAGAGCGCCCAATACGCAAACC
GCCTCTCCCCGCGCGTTGGCCGATTCATTAATG

1.-68. (canceled)

69. A method of modifying a cell, comprising introducing into a cell:
(a) a nuclease or a nucleic acid encoding the nuclease, wherein the nuclease is capable of cleaving a Wiskott-Aldrich Syndrome (WAS) locus in a cell genome; and
(b) a vector comprising a WAS gene or portion thereof.

70. The method of claim 69, wherein the nuclease comprises a Cas nuclease.

71. The method of claim 70, further comprising introducing into the cell a guide RNA (gRNA).

72. The method of claim 71, wherein the gRNA is capable of specifically hybridizing to a first exon in a WAS gene.

73. The method of claim 72, wherein the gRNA is encoded by a nucleotide sequence having at least 95% sequence identity to any one of SEQ ID NOs: 31-34.

74. The method of claim 69, wherein the vector is an adeno-associated viral (AAV) vector.

75. The method of claim 69, wherein the vector comprises a WAS cDNA.

76. The method of claim 75, wherein the vector lacks a promoter operably linked to the WAS cDNA.

77. The method of claim 75, wherein the WAS cDNA is inserted into a first exon of a WAS gene.

78. The method of claim 69, wherein the vector comprises an enhancer.

79. The method of claim 69, wherein the vector comprises a nucleic acid homologous to the WAS locus.

80. The method of claim 69, wherein the cell is selected from a T cell or a hematopoietic stem cell (HSC).

81. The method of claim 80, wherein the cell is a CD34+ HSC.

82. The method of claim 69, wherein the cell comprises a single X chromosome.

83. The method of claim 69, wherein the nuclease comprises a transcription activator-like effector nuclease (TALEN).

84. The method of claim 83, wherein the TALEN is encoded by a nucleotide sequence selected from any one of SEQ ID NOs: 27-30.

85. A cell prepared by the method of claim 69.

86. A method of treating, inhibiting or ameliorating Wiskott-Aldrich syndrome (WAS) or X-linked thrombocytopenia (XLT) in a subject, comprising: administering the cell of claim 85 to the subject.

87. The method of claim 86, wherein the cell is autologous to the subject.

88. The method of claim 86, wherein the cell is administered by adoptive cell transfer.


SEQUENCE LISTING (41 sequences)

SEQ ID NO: 1 - DNA, 2934 bp, synthetic construct
  misc_feature 1..2934: note = Sequence of Full chemically modified guide #1
  source 1..2934: mol_type = other DNA
atggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgactccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgactccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaa

SEQ ID NO: 2 - DNA, 3036 bp, synthetic construct
  misc_feature 1..3036: note = Sequence of Full chemically modified guide #1
  source 1..3036: mol_type = other DNA
atggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaa

SEQ ID NO: 3 - DNA, 20 bp, synthetic construct
  misc_feature 1..20: note = Sequence of Full chemically modified guide #1
  source 1..20: mol_type = other DNA
ggtatgttctgctgaaccgc

SEQ ID NO: 4 - DNA, 9634 bp, synthetic construct
  misc_feature 1..9634: note = Sequence of Full chemically modified guide #1
  source 1..9634: mol_type = other DNA
agcagaaggggttctgaacctaggttcaggagagaggctttgaacctgcacgtgtgggaagccatggaagtttccaggaaggactgcaggtcccaactggagatgtgccgttcctccttcaggtacctgggaatgtcagtcacaccccagacctgctcagctcccccaaactgctgttcctgtatctgagagcttcaagtctccaaatggcctacctcatacatggggaaactgaggcctggggaggccggggactgagctagcattcacttgtggaaatagtctggcatcatctggagaagttagagacatgcaaaccctacagccctcagattcccgtctgagagtctgcatgcctatgtggaccaggagatgtgtgcgggagtgaacactgcagtgttgctcccaacagcaagaaccagaagcagcccaaagggctgttacaggagaatatggacacccaggctgcacatgcacaccatggaatgctgtatggcagtggaaataaatgaacagctaccactataggcaaacaggaatcacagcaacagccaagagtgaaggcgtggagggacgagaccatgcactcacacctggcctgcctggctcgcactccgggcaaaggggtcagaacagtgactggcacacacgttaagtgctatgtgagtgttaagataaaactaggatgtccagtggggagaaagcaagcctttgaagattatgtgcttttacaaacttcaagtgcaatgaaaactaaacaagatgttgttcaggcattcatatatgatataaagttcctttctttaaaaaagggatgggctgggcacggtggctcacgcctgtaattctaatactttgggaggccgaggcaggtggatcacgaggtcgagaaatcgagaccatcctggccaacatggtgaaaccctgtctctactaaaaatacaaaaaaattagctgggcgtggtggcgtgtgcctgtagtcccagctacttgggaggctgaggcaggagagtcacttgaacccgggaggcaaaggttgcagtgagccgagatcgtgccaccgcactccagcctggcgacagagtgagactccatctcaaaaaaaaaaagaaaaaaaaaagtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgagtgggggcccaatgggaggaaggcccgggggccgaggagcaccagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctcctctcctagaatttcccgtcataatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttcccatattaaaattcctgaacactcaaataccgaggtagttcttaagcaaaaagtcttttccacaatcccctgacctgaactttctaggtttaagccccaaattcatccttttaaacccataaagatggacccagcataacttccagatcccaaggctatcaaatatccaccaaactcctaaaccataactctctccacaaaccccaaattgcacttactttagctggactccccgcgaaactcccaagtctatgtgtctgaacttcaaatctcaactccaacccccaaatactagaatcctacctgtcatgaattggggctggggtggtgggggagggcatggattgaatctgtgaatgagcctcaacttcctaagactagagtcctaaattatgaaattcaagcccccaagtcccagatctagggccccaaaccccaaatccaaacctctcacaaaagtgtatggctcccagactataccccacaatccacacccttagacaccaactctctggtgctgagctgaaaatctccaaaccagactatgaggctcccaaatccagacaccctgctccctgcccagctaacaaaagcctgccacccccggcgtgcctcagtgccactgtgcctcccaccctacacctctccaggctggtcggctgctctgggaacaggagctgtactcacagcttgtctactccacccccacccccttcttccacaccttcgctggagatgtaagtgatcaaccagccctcgggcctcacttggggtgtggagaggagatgggaaagttgcgggggacctgggaggcggctgaccccaaggtatgtgcaggactgccaagcggggctgaactttgcagacgaggacgaggcccaggccttccgggccctcgtgcaggagaagatacaaaaaaggaatcagaggcaaagtggaggtgaggaggccacaggggaggaaaggaagttgggcagaggtgagtgcaagcctggggaactagaaaagtcccctctcatggtcctggctcccaatccatctatccacagacagacgccagctacccccaccaccaacaccagccaatgaaggtgagtcctctagtgcaagtaggggtaataaggggctagcccaggaacctgtggcagggctgtgataactctctacacattccatcttcccagagagaagaggagggctcccacccctgcccctgcatccaggtggagaccaaggaggtgcgtgctgattcttccctgtgtctctggatggatgggtaagagtggatggaggaatgaggagttggatgggtgcgtaagtgggtgaatggataggtagattgataggtatgtggatggacgagcaggtgcatggatgtgtggactgatggatgggtggatggattggcggtagatggctgagtagagggatgaattgatgggaggatgaaagtctaagtagatagatgcataggtgaatgggtatgtggataaatgaatgaaaaggtagatggatgactgagtaaattaatcaatgagtgaatgaatgaacagtgaataaatgactaaatgacaagtttcagtcagtgaagaaagcatgattgaatgaataaatgagtaaatgaatattttaacaaattcattagtcaatgagccagtgaatgataaagcatgagggaatgaaaacatgaatgaatcagtgaatgtatgaatggtttgtgggatccacccacttctccatagaccctacttgaacccttcacccactacctccatgaccatccaacacacacacagatttccctcaaggcttccgtttcttgcccctgtgctttggttggttggtaagtgggtcaatgagccaaccaccctattttccccacaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagtgagagccactccccagtggacccacagattcctgggggcagaggggcacatgaacaagtggacagctgagtgaatggaaggatgggcagatgggcagatggctgggtggctgagtgggtaaatgggtggttggataggtaggtgcagggctgggtctagggagaggtaaataaggcaccaagggtacaaaatttaaggaggcactcactctcagaggcatgcaactgtaattcctgactctcagagtgagtgactcacttaaattttgcaccctaggcaccttacttgcctcaccctgggcccactctgggtgggctgttaggagagcaggtgggtgggcaggtgaacaaatggatagatagatgaggtagatgatggatgagaagggctggtgggtaggtgggtgagtggatgggtggatggatggataaatgaatggatgaatgaatgggttgaagaatgaatggataagtggttggatggacaagtttatgggtggatgggttgatgggaggtgcgtggatagatagatgggtgagtggataggtgtgtggacagattgatatgcaggctgattggctcacagacaaggtggatggggatggacaggtggacagatacgtggatgaatggacagttcaatggataagtgaacagaagtgtgtggttgcatgggtagaaaaatgagtggatggatagatggaaaggtgggcacatgggtaggtggatgggtggatggacaagtgtgtgtgaggacagactggtggacaaatgggtgaacagacatatgtgggcagatagttgcagagacagatgtatggacagatcagtagtccaacagatgaatgtgaatgaataggtggacaaatgcatgggatagatggggaaagagggatgggtggatggatcagcaccacaaactatggagcccttctaattccataactcctgcctatactcattcactcattcagtctcattcattaattctggcccctcagagtctctttgggcaggagagggcaagagggtttcactatgaagggagggaaggaagggcagtgaggattcactggagtctcttcacctctcccaggcatgtcagccacgtggggtgggacccccagaatggatttgacgtgagtaacttcagagtctcttggactccactaaacttccacccacccttccaaagaccactgctgagaccccacccccagatcgtgcccttcccacacccctctcagatcccttgctgggatggacccaacgacaatccatgtcgcttgtctcctcgccttattcctctactcctgcccctggcctttttcctcctgggcaggtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccagggtgagaccctgcttccatacgctcccttctctagcccaagcagctcatagctaagatacgcactaagtcactcagtccttatgggagcacctatactgcttcagtcaggagttggtcagtgggggtacccattttacaaatgagcaaaactgaggctcagaagaaatcaatgagagttacagctatgtgttataccccctccacagagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaaggtgaggacaggcaggatggaggattgggggtctaggactctggggtgtcccgtctaagtcaggatactggggggctgaggccaggactgaggagagtgccaggccttagggattcagtgatagggttgaaaggttggtgggaagccttgaaggggactggagtgtgtgggagagaaaatattgatggaggggcggggagaaatgctcctttcccaggccctaagccctctgtgctgatccctgcctgctgcagacccctggggccccagagagctcagcgctgcagccaccacctcagagctcagagggactggtgggggccctgatgcacgtgatgcagaagagaagcagagccatccactcctccggtgagctgatcctgccggggcctcaaacctggctcccagggctagcactggcctcaaaacaatcccagcagtcaccaccaatagtgacatcagccccatctgtttgacagcattaacatgaatcttgtgtcagcctcgtttttgacaatgttaacattaagtcattatgtgacaataatataattaactccaactttgacagtaatattaacattaatgccagggtgtgtccacaatattaatgtcattcccacatgttcagtactactaacatcagctggccgggcgcggtggctcatgcctgtaatccaggaactttgggaggctaaggcaggaggatcacttgagcccaggagttcgagaccagcctgggcaatatagtgagacctcgtttccataaaaactaaattcaaaaaaagtagtcaagcatagtggtgtgtgcctgtggtcccagctacttgggaggctgaggtgggaggattgcttgaccctgggaggtcaaggcagcagtgatccatgattgtgccactgcactccagcctgggtgacagagatcctatctcaaaaaaaaaaaaaattaacccattatgtgatgacaatattatgaagaacactattgttgacaatattaattttaattccatgtattaacagatttacattaattcattatgacgtaacctaatctaatcttttaaaaaatttttttgaaacagggtctcgctctgtgtcccaggctggagtacagtggtgcaatcatggctcagtgcagcctcaacctcccaggctcaagcgatcctcccgcctcagctcccaaagtagctgggactacaggcgtgtgccaccatacctggctaatttttggtgtttttttggtagtgatgagctctcactaccaagctctcactactctcatgttgcccaggctgctctgcaactccagggctcaagcgatctgccccgcctcagcctcccagagtgctgggattacaggcatgagccaccaggcctggctgttaacctaatctttttataataatgttactattactctcttaatctgtcagcaatactgtcactaatccattatatgatgcaaatattagtatcaacctactataggaacttcatctttcgacaatgatttttttttttcttttgagacggagtcttgctctgtcacccaggctggagtgcagtggcgcgatcttggctaactgcagactctgcctcctgggttcaagcgattctcctgcctcagccttccgagtagctgggactacaggcacgccactacgcccagctaattttgatgttattgtcattaaccccattatgtgtcaaaaatattagcgttaaccagacaggaagcaataatatattatcacacctttgctaatattatttaaattcaccctattatgtgataaataggttaacattaaccctttgtttgacaatatctcgactaaccacatttttgacagcataaacttcaactccaactagaactcagaccccaactataatccctttcttgtcccaaatggaaactctaacttgccctcctctagcatgagacctcagaaccccagggtccagtcctcacctcccaggccctatgaagccccccaccaacctcccagggcatcttatctttctctttccctccagacgaaggggaggaccaggctggcgatgaagatgaagatgatgaatgggatgactgagtggctgagttacttgctgccctgtgctcctccccgcaggacatggctccccctccacctgctctgtgcccaccctccactctcctcttccaggcccccaaccccccatttcttccccaccaacccctccaatgctgttatccctgcctggtcctcacactcacccaacaatcccaaggccctttttatacaaaaattctcagttctcttcactcaaggatttttaaagaaaaataaaagaattgtctttctgtctctctataaa

SEQ ID NO: 5 - DNA, 7264 bp, synthetic construct
  misc_feature 1..7264: note = Sequence of Full chemically modified guide #1
  source 1..7264: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtgtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgagtgggggcccaatgggaggaaggcccgggggccgaggagcacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaacagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctcctctcctagaatttcccgtcataatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttcccatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttgccgattcattaatg

SEQ ID NO: 6 - DNA, 7265 bp, synthetic construct
  misc_feature 1..7265: note = Sequence of Full chemically modified guide #1
  source 1..7265: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtgtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgagtgggggcccaatgggaggaaggcccgggggccgaggagcacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaacagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctccactcctagaatttcccgtcataatccacccttcccaggaagatctcaatgtcttcttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttcccatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 7 - DNA, 7047 bp, synthetic construct
  misc_feature 1..7047: note = Sequence of Full chemically modified guide #1
  source 1..7047: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtgtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgagtgggggcccaatgggaggaaggcccgggggccgaggagcacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaaaatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttcccatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 8 - DNA, 3451 bp, synthetic construct
  misc_feature 1..3451: note = Sequence of Full chemically modified guide #1
  source 1..3451: mol_type = other DNA
aagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcgatttaaattcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtatgttctgctgaaccgcgttttagagctagaaatagcaagttaaaataaggctagtccgttatcaacttgaaaaagtggcaccgagtcggtgctttttttctagacccagctttcttgtacaaagttggcattaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctaga

SEQ ID NO: 9 - DNA, 7691 bp, synthetic construct
  misc_feature 1..7691: note = Sequence of Full chemically modified guide #1
  source 1..7691: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtatgttctgctgaaccgcgttttagagctagaaatagcaagttaaaataaggctagtccgttatcaacttgaaaaagtggcaccgagtcggtgcttttttacgcgtgtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgagtgggggcccaatgggaggaaggcccgggggccgaggagcacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaacagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctcctctcctagaatttcccgtcataatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttcccatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 10 - RNA, 100 nt, synthetic construct
  misc_feature 1..100: note = Sequence of Full chemically modified guide #1
  source 1..100: mol_type = other RNA
ggtatgttctgctgaaccgcgttttagagctagaaatagcaagttaaaataaggctagtccgttatcaacttgaaaaagtggcaccgagtcggtgctttt

SEQ ID NO: 11 - DNA, 984 bp, synthetic construct
  misc_feature 1..984: note = Upstream homology arm (common in #1201, #1262 and#1244)
  source 1..984: mol_type = other DNA
gtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgagtgggggcccaatgggaggaaggcccgggggccgaggagcac

SEQ ID NO: 12 - DNA, 1000 bp, synthetic construct
  misc_feature 1..1000: note = Downstream homology arm for #1201
  source 1..1000: mol_type = other DNA
cagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctcctctcctagaatttcccgtcataatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttccca

SEQ ID NO: 13 - DNA, 1000 bp, synthetic construct
  misc_feature 1..1000: note = Downstream homology arm #1244
  source 1..1000: mol_type = other DNA
cagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctccactcctagaatttcccgtcataatccacccttcccaggaagatctcaatgtcttcttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttccca

SEQ ID NO: 14 - DNA, 782 bp, synthetic construct
  misc_feature 1..782: note = Downstream homology arm #1262
  source 1..782: mol_type = other DNA
aatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttccca

SEQ ID NO: 15 - DNA, 17 bp, synthetic construct
  misc_feature 1..17: note = WAS TALEN binding site on the genome(forward-TALEN)
  source 1..17: mol_type = other DNA
ctcctagaatttcccgt

SEQ ID NO: 16 - DNA, 18 bp, synthetic construct
  misc_feature 1..18: note = WAS TALEN binding site on the genome(reverse-TALEN)
  source 1..18: mol_type = other DNA
aggaagatctcaatgtct

SEQ ID NO: 17 - DNA, 17 bp, synthetic construct
  misc_feature 1..17: note = TALEN cleavage site (spacer sequence)
  source 1..17: mol_type = other DNA
cataatccacccttccc

SEQ ID NO: 18 - DNA, 141 bp, synthetic construct
  misc_feature 1..141: note = WAS exon 2
  source 1..141: mol_type = other DNA
acgctggccactgcagttgttcagctgtacctggcgctgccccctggagctgagcactggaccaaggagcattgtggggctgtgtgcttcgtgaaggataacccccagaagtcctacttcatccgcctttacggccttcag

SEQ ID NO: 19 - DNA, 189 bp, synthetic construct
  misc_feature 1..189: note = WAS exon 1 sequence
  source 1..189: mol_type = other DNA
tcctcttcttaccctgcacccagagcctcgccagagaagacaagggcagaaagcaccatgagtgggggcccaatgggaggaaggcccgggggccgaggagcaccagcggttcagcagaacataccctccaccctcctccaggaccacgagaaccagcgactctttgagatgcttggacgaaaatgcttg

SEQ ID NO: 20 - DNA, 7468 bp, synthetic construct
  misc_feature 1..7468: note = pWNY.2xNLS.Cas9.mCherry
  source 1..7468: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatacacttagtgtaatacgactcactatagggagagcggccgctttttcagcaagattaagccgccaccatggcgccgcggcctcctaagaagaagcggaaagtcgaattctacgtaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagacaatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtgtccgatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggctctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcggaagcggaggtgagggcagaggaagtcttctaacatgcggtgacgtggaggagaatccgggccctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaggtacccgtacgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 3452 bp, synthetic construct
  misc_feature 1..3452: note = 1190 scAAV.U6.guideRNA2
  source 1..3452: mol_type = other DNA
aagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcgatttaaattcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaagagtcgctggttctcggttttagagctagaaatagcaagttaaaataaggctagtccgttatcaacttgaaaaagtggcaccgagtcggtgctttttttctagacccagctttcttgtacaaagttggcattaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctaga

SEQ ID NO: 22 - DNA, 3452 bp, synthetic construct
  misc_feature 1..3452: note = #1191 scAAV.U6.guideRNA3
  source 1..3452: mol_type = other DNA
aagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcgatttaaattcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaagcatctcaaagagtcgcgttttagagctagaaatagcaagttaaaataaggctagtccgttatcaacttgaaaaagtggcaccgagtcggtgctttttttctagacccagctttcttgtacaaagttggcattaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctaga

SEQ ID NO: 23 - DNA, 3452 bp, synthetic construct
  misc_feature 1..3452: note = #1192 scAAV.U6.guideRNA4
  source 1..3452: mol_type = other DNA
aagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcgatttaaattcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccatgagtgggggcccaatgttttagagctagaaatagcaagttaaaataaggctagtccgttatcaacttgaaaaagtggcaccgagtcggtgctttttttctagacccagctttcttgtacaaagttggcattaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctaga

SEQ ID NO: 24 - DNA, 3036 bp, synthetic construct
  misc_feature 1..3036: note = WAS TALEN #2 forward
  source 1..3036: mol_type = other DNA
atggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagcaacaatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagcaacaatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaa

SEQ ID NO: 25 - DNA, 2934 bp, synthetic construct
  misc_feature 1..2934: note = WAS TALEN #2 Reverse
  source 1..2934: mol_type = other DNA
atggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgactccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaa

SEQ ID NO: 26 - DNA, 7084 bp, synthetic construct
  misc_feature 1..7084: note = #1380 AAV.WASATGcoWAS.WPRE3.pA
  source 1..7084: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgggaggaagacccggcggccgaggagcgccagcagtgcaacaaaacattccgtcaaccctgctgcaggaccacgaaaaccagaggctgtttgaaatgttgggacggaagtgtctcactctcgccacagccgtcgtccagctttatcttgcgcttcctcccggtgctgagcattggactaaagagcattgcggcgcggtctgttttgtcaaggataatccccaaaaatcatatttcattaggttgtacggactccaagctggacgccttctgtgggaacaagaactctatagccagctcgtatatagcacaccgacccctttcttccatactttcgcgggagacgactgtcaggcgggcttgaactttgcggacgaggatgaagctcaggctttccgagcattggttcaagaaaaaatccagaaaagaaatcagcgacagtccggagatcgccggcagctgccgccgccacctacaccggccaatgaggaacggaggggaggccttccgccacttccattgcatccaggcggcgatcagggtgggccaccagtagggcccttgagtttgggtctcgctactgtggatatacagaacccggacataacatctagccgctaccgcggactgccggctccaggtccgtcccccgctgataaaaagcgctccggcaaaaagaagatatctaaagcagatatcggtgcgccctccggtttcaagcatgtctcccatgtaggatgggacccgcaaaatggattcgacgttaataacctcgatccggacctgaggagtctcttctctcgcgcgggtatcagcgaggcacagcttactgatgccgaaacaagtaagttgatatacgactttatcgaggatcaaggagggctggaagcggtcaggcaagaaatgcggcgacaagaacctttgcccccgcccccgcccccgtccagaggcgggaaccagcttccacgcccacctatcgttggagggaataaaggcaggtctgggccactccctccggtaccgttggggatcgctccaccgcctcctacgcctaggggacccccgcctcctggtcgggggggaccgccccctccgccgcctccagccactggtcgaagtggacccctcccgcctcctccacccggcgccgggggcccaccgatgccacctcctcctccgcccccaccgcctcccccttcttccggcaacggtcccgcacctccgcccctccctccggcattggtccccgcggggggcctcgcgcctggtggtggccggggtgcacttctggatcaaatccgacagggcatacagttgaataagacgcccggcgcccctgaaagctcagctctgcaaccgccgcctcagtcctctgaagggttggtaggcgcgctcatgcatgtaatgcagaagcgcagtcgcgctatccactcatcagatgaaggtgaagaccaggccggtgacgaggacgaagacgatgaatgggacgattgactgaactgaactagtgtcgacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggaggtgtgggaggttttttaaacagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctcctctcctagaatttcccgtcataatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 27 - DNA, 17 bp, synthetic construct
  misc_feature 1..17: note = WAS TALEN#1forward RVD sequence
  source 1..17: mol_type = other DNA
ctcctagaatttcccgt

SEQ ID NO: 28 - DNA, 18 bp, synthetic construct
  misc_feature 1..18: note = WAS TALEN#1 reverse RVD sequence
  source 1..18: mol_type = other DNA
agacattgagatcttcct

SEQ ID NO: 29 - DNA, 18 bp, synthetic construct
  misc_feature 1..18: note = WAS TALEN#2 forward RVD sequence
  source 1..18: mol_type = other DNA
ctggctgcagctcttcct

SEQ ID NO: 30 - DNA, 17 bp, synthetic construct
  misc_feature 1..17: note = WAS TALEN#2 reverse RVD sequence
  source 1..17: mol_type = other DNA
ggtccttcctgcctcat

SEQ ID NO: 31 - DNA, 20 bp, synthetic construct
  misc_feature 1..20: note = WAS GUIDE#1 sequence
  source 1..20: mol_type = other DNA
ggtatgttctgctgaaccgc

SEQ ID NO: 32 - DNA, 20 bp, synthetic construct
  misc_feature 1..20: note = WAS GUIDE#2 sequence
  source 1..20: mol_type = other DNA
caaagagtcgctggttctcg

SEQ ID NO: 33 - DNA, 20 bp, synthetic construct
  misc_feature 1..20: note = WAS GUIDE#3 sequence
  source 1..20: mol_type = other DNA
caagcatctcaaagagtcgc

SEQ ID NO: 34 - DNA, 20 bp, synthetic construct
  misc_feature 1..20: note = WAS GUIDE#4 sequence
  source 1..20: mol_type = other DNA
accatgagtgggggcccaat

SEQ ID NO: 35 - DNA, 7265 bp, synthetic construct
  misc_feature 1..7265: note = pAAV.DT (#1201)
  source 1..7265: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtgtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgagtgggggcccaatgggaggaaggcccgggggccgaggagcacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaacagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctcctctcctagaatttcccgtcataatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagacctcagacctgatcagtgaatccctgagccccagaaccaaagactcatccagatggcaaactctgacttgcctttctaagtctgcaatgactggccccagtctccgtatcaagatctctaaagcccccagtattagtctgctgcctaagcctaatcttttccacaaattccaataaatgagcactgtatttgtacctgaacctcaaatctattctaaactcaacattttgcatcccaggaatctctcatcaaaactcctgaaccccagatgtttgccaagctcctaagtcataaatctgttcaacaaaccccaaagttgaatattccattgatccttgaactccaaatctgtccttctaaatccacagcacagaccccagagttcccatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 36 - DNA, 7265 bp, synthetic construct; identical to SEQ ID NO: 6
  misc_feature 1..7265: note = pAAV.DT-M (#1244)
  source 1..7265: mol_type = other DNA

SEQ ID NO: 37 - DNA, 7047 bp, synthetic construct; identical to SEQ ID NO: 7
  misc_feature 1..7047: note = pAAV.DT-D (#1262)
  source 1..7047: mol_type = other DNA

SEQ ID NO: 38 - DNA, 3451 bp, synthetic construct; identical to SEQ ID NO: 8
  misc_feature 1..3451: note = pscAAV.G(#1189)
  source 1..3451: mol_type = other DNA

SEQ ID NO: 39 - DNA, 7691 bp, synthetic construct; identical to SEQ ID NO: 9
  misc_feature 1..7691: note = pAAV.DTG(#1215)
  source 1..7691: mol_type = other DNA

SEQ ID NO: 40 - DNA, 6289 bp, synthetic construct
  misc_feature 1..6289: note = pAAV.ATG.GFP (#1374)
  source 1..6289: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgtaggctcgtcttgaactcctgacctcaagtgatccactcgtctcggcctcccaaagtgctgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggaggtgtgggaggttttttaaacagcggttcagcagaacataccctccaccctcctccaggaccacgagaaccagcgactctttgagatgcttggacgaaaatgcttggtgagctggggatctcctgcccccgccccgtccccaccgtttcttcctcttcctctcctccttctctctcttcccctcctcccgctcctcctttccctctccatcatctcctctcctagaatttcccgtcataatccacccttcccaggaagatctcaatgtctacttgccttccctctggctgcagctcttcctttgggcccatgactgtcatgaggcaggaaggaccaggtctggctccaagaccttgtggctacccctgaccagactccactgacccctgctttcctctcccagacgctggccactgcagttgttcagctgtacctggcgctgccccctggagctgagcactggaccaaggagcattgtggggctgtgtgcttcgtgaaggataacccccagaagtcctacttcatccgcctttacggccttcaggtgacccccccacccccgactggacttgcaagccagttctcaacccgcaaacccagatctgtgtccatatgtgtccatagcttcaagtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 41 - DNA, 7084 bp, synthetic construct; identical to SEQ ID NO: 26
  misc_feature 1..7084: note = pAAV.ATG.coWAS (#1380)
  source 1..7084: mol_type = other DNA